(19) United States
(12) Patent Application Publication
(10) Pub. No.: US 2021/0345601 A1
(43) Pub. Date: Nov. 11, 2021

(54) ENZYMATIC COMPOSITIONS FOR CARBOHYDRATE ANTIGEN CLEAVAGE ON DONOR ORGANS, METHODS AND USES ASSOCIATED THEREWITH

(71) Applicants: THE UNIVERSITY OF BRITISH COLUMBIA, Vancouver (CA); UNIVERSITY HEALTH NETWORK, Toronto (CA)

(72) Inventors: Marcelo CYPEL, Toronto (CA); Aizhou WANG, Toronto (CA); Shafique KESHAVJEE, Toronto (CA); Stephen G. WITHERS, Vancouver (CA); Peter RAHFELD, Vancouver (CA); Jayachandran KIZHAKKEDATHU, New Westminster (CA)

(73) Assignees: THE UNIVERSITY OF BRITISH COLUMBIA, Vancouver, BC (CA); UNIVERSITY HEALTH NETWORK, Toronto, ON (CA)

(21) Appl. No.: 17/269,238

(22) PCT Filed: Aug. 16, 2019
(86) PCT No.: PCT/CA2019/051121

§ 371 Date: Feb. 17, 2021

Related U.S. Application Data
(60) Provisional application No. 62/719,272, filed on Aug. 17, 2018.

(51) Int. Cl. A01N 1/02; C12N 9/80; C12N 9/24

(52) U.S. Cl. CPC A01N 1/0226; C12N 9/80; C12Y 302/01049; C12N 9/2402; C12Y 305/01025

(57) ABSTRACT

Provided herein are perfusion fluids for enzymatically cleaving A-antigens from a donor organ, and methods, uses, associated therewith. In particular, the perfusion fluids comprise two enzymes, GalNAcDeacetylase and Galactosaminidase and the fluids may further comprise a buffered extracellular solution and/or a crowing agent. Furthermore, the compositions described herein were found to have activity at temperatures and pH levels suitable for cell viability.

### CROSS REFERENCE TO RELATED APPLICATION

This application claims the benefit of U.S. Provisional Patent Application Ser. No. 62/719,272 filed on 17 Aug. 2018, entitled “ENZYMATIC COMPOSITIONS FOR CARBOHYDRATE ANTIGEN CLEAVAGE, METHODS, USES, APPARATUSES AND SYSTEMS ASSOCIATED THEREWITH”.

### TECHNICAL FIELD

The present invention relates to the field of enzyme compositions. In particular, the invention relates to enzyme compositions for cleaving antigens on donor organs, and providing methods and uses for cleaving antigens using the compositions.

### BACKGROUND

Correct matching of blood types is a central requirement of transfusion medicine since plasma of blood group A individuals contains antibodies to the B-antigen and vice versa, thus incompatible transfusions can result in activation of complement and red blood cell (RBC) lysis (Daniels 2010). These cell surface antigens are carbohydrate structures terminating in α-1,3-linked-N-acetylgalactosamine (GalNAc) or galactose (Gal) for A-type blood and B-type blood respectively. 0 type RBCs, on the other hand, contain neither of these terminal sugars, and may be transfused universally (Garratty 2008). Accordingly, a good supply of group O RBCs is needed in blood banks for emergency situations, where the patient's blood type is unknown or unclear. However, supplies are often limited.

The concept of enzymatic removal of the GalNAc or Gal structures from A or B RBCs as a means of converting A or B RBCs to O was first proposed and demonstrated by Goldstein (Goldstein 1982; US4609627; and CA2272925). Using an α-galactosidase from green coffee bean, B-type RBCs were converted to 0 and subsequent successful transfusion performed (Kruskall 2000). However, the quantities of enzyme that were needed, rendering the approach impractical. Conversion of Type A is more challenging, largely because Type A blood occurs as many subtypes that differ in their internal linkages (Clausen 1989). Similarly, α-galactosidases have been used to remove B-type antigens (for example, see EP2243793) A major advance towards practical conversions, including of Type A, was made by screening of a library of bacteria for both A and B conversion activities, using tetrasaccharide substrates. Two new families of glycosidase were found that show high antigen cleavage activity at neutral pH values: the CAZy GH109 α-N-acetylgalactosaminidases and the GH110 α-galactosidases (Liu 2007). Both enzymes converted their corresponding RBCs with complete removal of the respective antigens. However, substantial amounts of enzyme were still needed for conversion, especially of Type A (60 mg enzyme/unit of blood), limiting further development. Enzymes having greater efficiency in cleaving the carbohydrate antigens from cells would be of use.

### SUMMARY

The present invention is based in part, on the surprising discovery that the combination of a Galactosaminidase and a GalNAcDeacetylase, as described herein, are orders of magnitude more efficient than previously identified A-antigen cleaving enzymes. For example, under some conditions some of the GalNAcDeacetylase and Galactosaminidase enzymes may be capable of cleaving A-antigen at or below 1μ/ml. Furthermore, the cleavage efficiency of the enzyme combination is maintained at a pH suitable to maintain viability of the erythrocytes (i.e. pH between about 6.5 and about 7.5). Additionally, the enzymes were found to be active at temperatures between 4° C. and 37° C., which is also suitable for blood collection, washing and storage protocols. Furthermore, the efficiency of the enzymes is further improved through the addition of a crowding agent (for example, dextran). It has also been appreciated that the same two step cleavage process could be applied to donor organs.

However, it will be appreciated by a person of skill in the art that more enzyme could be used to reduce the time in which the donor organ may be perfused or less enzyme could be used, provided that the donor organ is perfused longer.

In accordance with one embodiment, there is provided a perfusion fluid for enzymatically cleaving A-antigens from a donor organ including: (a) a purified GalNAcDeacetylase protein; and (b) a purified Galactosaminidase protein.

In accordance with a further embodiment, there is provided a perfusion fluid wherein the perfusion fluid includes: (a) the GalNAcDeacetylase is a purified protein selected from one or more of: SEQ ID NO.:2; SEQ ID NO.:4; SEQ ID NO.:5; SEQ ID NO.:17; SEQ ID NO.:23; SEQ ID NO.:29; SEQ ID NO.:31; SEQ ID NO.:32; SEQ ID NO.:33; SEQ ID NO.:34; and SEQ ID NO.:35; and (b) the Galactosaminidase is a purified protein is selected from one or more of the following: SEQ ID NO.:7; SEQ ID NO.:9; SEQ ID NO.:10; SEQ ID NO.:19; SEQ ID NO.:21; SEQ ID NO.:36; and SEQ ID NO.:37.

In accordance with a further embodiment, there is provided a perfusion fluid wherein the perfusion fluid includes: a purified enzyme having a GalNAcDeacetylase activity consisting essentially of an amino acid sequence at least 90% identical to the sequence set forth in one of SEQ ID NOs:2, 4, 5, 17, 23, 29, 31 and 32-35; and a purified enzyme having Galactosaminidase activity consisting essentially of an amino acid sequence at least 90% identical to the sequence set forth in one of SEQ ID NOs:7, 9, 10, 19, 21, 36 and 37.

The enzymes may be selected from one or more of: (a) the purified GalNAcDeacetylase protein is a purified Flavonifractor plautii GalNAcDeacetylase protein of SEQ ID NO.:2, SEQ ID NO.:4 and SEQ ID NO.:5; and (b) the purified Galactosaminidase protein is a purified Flavonifractor plautii Galactosaminidase protein of SEQ ID NO.:7, SEQ ID NO.:9 and SEQ ID NO.:10. The enzymes may be selected from one or more of: (a) the purified GalNAcDeacetylase protein is a purified Clostridium tertium GalNAcDeacetylase protein of SEQ ID NO.:17 or SEQ ID NO.:32; and (b) the purified Galactosaminidase protein is a purified Clostridium tertium Galactosaminidase protein of SEQ ID NO.:19 or SEQ ID NO.:36. The GalNAcDeacetylase and the Galactosaminidase may be capable of cleaving A-antigen at or below 1 μg/ml. The GalNAcDeacetylase and the Galactosaminidase may have A-antigen cleaving activity at a pH between about 6.5 and about 7.5. The GalNAcDeacetylase and the Galactosaminidase may have A-antigen cleaving activity at a temperatures between 4° C. and 37° C. The perfusion fluid may further include a buffered extracellular solution. The buffered extracellular solution may be selected from: Steen™; Perfadex™; Perfadex Plus™; EuroCollins solution; Histidine-Tryptophan-Ketoglutarate (HTK) solution; University of Wisconsin solution (UW); Celsior solution; Kidney Perfusion solution (KPS-1); Kyoto University solution; IGL-1 solution; and Citrate solution.

In accordance with a further embodiment, there is provided a method for enzymatically cleaving A-antigens ex vivo from a donor organ, the method including: (a) perfusing a donor organ displaying type A antigen with a fluid comprising GalNAcDeacetylase protein and a Galactosaminidase protein for a period of time sufficient to allow the enzymes to cleave A-antigens from the donor organ; or (b) incubating a donor organ displaying type A antigen with a fluid comprising GalNAcDeacetylase protein and a Galactosaminidase protein for a period of time sufficient to allow the enzymes to cleave A-antigens from the donor organ.

The GalNAcDeacetylase may be a purified protein selected from one or more of: SEQ ID NO.:2; SEQ ID NO.:4; SEQ ID NO.:5; SEQ ID NO.:17; SEQ ID NO.:23; SEQ ID NO.:29; SEQ ID NO.:31; SEQ ID NO.:32; SEQ ID NO.:33; SEQ ID NO.:34; and SEQ ID NO.:35; and the Galactosaminidase may be a purified protein is selected from one or more of the following: SEQ ID NO.:7; SEQ ID NO.:9; SEQ ID NO.:10; SEQ ID NO.:19; SEQ ID NO.:21; SEQ ID NO.:36; and SEQ ID NO.:37.

The purified enzyme having the GalNAcDeacetylase activity may include essentially an amino acid sequence at least 90% identical to the sequence set forth in one of SEQ ID NOs:2, 4, 5, 17, 23, 29, 31 and 32-35; and the purified enzyme having the Galactosaminidase activity may include essentially an amino acid sequence at least 90% identical to the sequence set forth in one of SEQ ID NOs:7, 9, 10, 19, 21, 36 and 37.

The GalNAcDeacetylase may be a purified Flavonifractor plautii GalNAcDeacetylase protein of SEQ ID NO.:4 or SEQ ID NO.:5 and the Galactosaminidase may be a purified Flavonifractor plautii Galactosaminidase protein of SEQ ID NO.:9 or SEQ ID NO.: 10.

The GalNAcDeacetylase protein and the Galactosaminidase protein may be in a buffered extracellular solution. The buffered extracellular solution may be selected from: Steen™; Perfadex™; Perfadex Plus™; EuroCollins solution; Histidine-Tryptophan-Ketoglutarate (HTK) solution; University of Wisconsin solution (UW); Celsior solution; Kidney Perfusion solution (KPS-1); Kyoto University solution; IGL-1 solution; and Citrate solution. The donor organ may be a solid organ. The solid organ may be selected from one of the following: lung; kidney; liver; heart; pancreas; and intestine. The solid organ may be a lung.

The GalNAcDeacetylase protein and the Galactosaminidase protein may be mixed with an ex vivo buffered extracellular lung solution and circulated through the lung, whereby the GalNAcDeacetylase protein and the Galactosaminidase protein are in contact with the vasculature of the donor organ for a period of time sufficient to substantially clear the A-antigens from the vasculature of the lung. The GalNAcDeacetylase protein and the Galactosaminidase protein may be mixed with an ex vivo buffered extracellular kidney solution and circulated through the kidney, whereby the GalNAcDeacetylase protein and the Galactosaminidase protein are in contact with the vasculature of the donor organ for a period of time sufficient to substantially clear the A-antigens from the vasculature of the kidney. The GalNAcDeacetylase protein and the Galactosaminidase protein may be mixed with an ex vivo buffered extracellular liver solution and circulated through the liver, whereby the GalNAcDeacetylase protein and the Galactosaminidase protein are in contact with the vasculature of the donor organ for a period of time sufficient to substantially clear the A-antigens from the vasculature of the liver. The GalNAcDeacetylase protein and the Galactosaminidase protein may be mixed with an ex vivo buffered extracellular heart solution and circulated through the heart, whereby the GalNAcDeacetylase protein and the Galactosaminidase protein are in contact with the vasculature of the donor organ for a period of time sufficient to substantially clear the A-antigens from the vasculature of the heart. The GalNAcDeacetylase protein and the Galactosaminidase protein may be mixed with an ex vivo buffered extracellular pancreas solution and circulated through the pancreas, whereby the GalNAcDeacetylase protein and the Galactosaminidase protein are in contact with the vasculature of the donor organ for a period of time sufficient to substantially clear the A-antigens from the vasculature of the pancreas. The GalNAcDeacetylase protein and the Galactosaminidase protein may be mixed with an ex vivo buffered extracellular intestine solution and circulated through the intestine, whereby the GalNAcDeacetylase protein and the Galactosaminidase protein are in contact with the vasculature of the donor organ for a period of time sufficient to substantially clear the A-antigens from the vasculature of the intestine.

The time to clear the A-antigens from the vasculature may be about 1 hour. The time to clear the A-antigens from the vasculature may be under 1 hour. The time to clear the A-antigens from the vasculature may be about 2 hours.

The method may further include washing the donor organ to remove GalNAcDeacetylase, Galactosaminidase and cleaved A-antigens. The GalNAcDeacetylase and Galactosaminidase may be capable of cleaving A-antigen at or below 1 μg/ml. The GalNAcDeacetylase and Galactosaminidase may have A-antigen cleaving activity at a pH between about 6.5 and about 7.5. The GalNAcDeacetylase and Galactosaminidase may have A-antigen cleaving activity at a temperatures between 4° C. and 37° C.

In accordance with a further embodiment, there is provided a composition, the composition including: a purified enzyme having a GalNAcDeacetylase activity consisting essentially of an amino acid sequence at least 85% identical to the sequence set forth in one of SEQ ID NOs:2, 4, 5, 17, 23, 29, 31 and 32-35; and a purified enzyme having Galactosaminidase activity consisting essentially of an amino acid sequence at least 85% identical to the sequence set forth in one of SEQ ID NOs: 7, 9, 10, 19, 21, 36 and 37.

In accordance with a further embodiment, there is provided a composition, the composition including: a purified enzyme having a GalNAcDeacetylase activity consisting essentially of an amino acid sequence at least 80% identical to the sequence set forth in one of SEQ ID NOs:2, 4, 5, 17, 23, 29, 31 and 32-35; and a purified enzyme having Galactosaminidase activity consisting essentially of an amino acid sequence at least 80% identical to the sequence set forth in one of SEQ ID NOs:7, 9, 10, 19, 21, 36 and 37.

In accordance with a further embodiment, there is provided a composition, the composition including: a purified enzyme having a GalNAcDeacetylase activity consisting essentially of an amino acid sequence at least 75% identical to the sequence set forth in one of SEQ ID NOs:2, 4, 5, 17, 23, 29, 31 and 32-35; and a purified enzyme having Galactosaminidase activity consisting essentially of an amino acid sequence at least 75% identical to the sequence set forth in one of SEQ ID NOs:7, 9, 10, 19, 21, 36 and 37.

The composition may include: (a) the purified GalNAcDeacetylase and the purified Galactosaminidase may be immobilized; (b) the purified GalNAcDeacetylase may be immobilized; or (c) the purified Galactosaminidase may be immobilized.

The immobilized enzymes may be attached to a surface, the surface may be selected from one or more of the following: (a) a bead or microsphere; (b) a container; (c) a tube; (d) a column; and (e) a matrix. The composition may further include a crowding agent. The crowding agent may be selected from one or more of: a dextran, a dextran sulfate, a dextrin, a pullulan, a poly(ethylene glycol), a Ficoll™, and an inert protein.

In accordance with a further embodiment, there is provided a purified enzyme including a Flavonifractor plautii GalNAcDeacetylase of SEQ ID NO.:2, SEQ ID NO.:4 or SEQ ID NO.:5.

In accordance with a further embodiment, there is provided a purified enzyme including a Flavonifractor plautii Galactosaminidase of SEQ ID NO.:7, SEQ ID NO.:9 or SEQ ID NO.:10.

In accordance with a further embodiment, there is provided a purified enzyme including a Clostridium tertium GalNAcDeacetylase of SEQ ID NO.:17 or SEQ ID NO.:32.

In accordance with a further embodiment, there is provided a purified enzyme including a Clostridium tertium Galactosaminidase of SEQ ID NO.:19 or SEQ ID NO.:36.

The protein tag may be selected from one or more of: Albumin-binding protein (ABP); Alkaline Phosphatase (AP); AUi epitope; AU5 epitope; AviTag; Bacteriophage T₇ epitope (T₇-tag); Bacteriophage V₅ epitope (V₅-tag); Biotin-carboxy carrier protein (BCCP); Bluetongue virus tag (B-tag); single-domain camelid antibody (C-tag); Calmodulin binding peptide (CBP or Calmodulin-tag); Chloramphenicol Acetyl Transferase (CAT); Cellulose binding domain (CBP); Chitin binding domain (CBD); Choline-binding domain (CBD); Dihydrofolate reductase (DHFR); DogTag; E2 epitope; E-tag; FLAG epitope (FLAG-tag); Galactose-binding protein (GBP); Green fluorescent protein (GFP); Glu-Glu (EE-tag); Glutathione S-transferase (GST); Human influenza hemagglutinin (HA); HaloTag™; Alternating histidine and glutamine tags (HQ tag); Alternating histidine and asparagine tags (HN tag); Histidine affinity tag (HAT); Horseradish Peroxidase (HRP); HSV epitope; Isopeptag (Isopep-tag); Ketosteroid isomerase (KSI); KT3 epitope; LacZ; Luciferase; Maltose-binding protein (MBP); Myc epitope (Myc-tag); NE-tag; NusA; PDZ domain; PDZ ligand; Polyarginine (Arg-tag); Polyaspartate (Asp-tag); Polycysteine (Cys-tag); Polyglutamate (Glu-tag); Polyhistidine (His-tag); Polyphenylalanine (Phe-tag); Profinity eXact; Protein C; Rho1D4-tag; S1-tag; S-tag; Softag 1; Softag 3; SnoopTagJr; SnoopTag; Spot-tag; Spy Tag (Spy-tag); Streptavadin-binding peptide (SBP); Staphylococcal protein A (Protein A); Staphylococcal protein G (Protein G); Strep-tag; Streptavadin (SBP-tag); Strep-tag II; Sdy-tag; Small Ubiquitin-like Modifier (SUMO); Tandem Affinity Purification (TAP); T₇ epitope; tetracysteine tag (TC tag); Thioredoxin (Trx); TrpE; Ty tag; Ubiquitin; Universal; V₅ tag; VSV-G or VSV-tag; and Xpress tag.

In accordance with a further embodiment, there is provided a method for enzymatically cleaving A-antigens from a donor organ, the method including: (a) combining a GalNAcDeacetylase protein and a Galactosaminidase protein with a donor organ displaying type A antigen; (b) perfusing the enzymes into the donor organ blood vessels for a period of time sufficient to allow the enzymes to cleave A-antigens from the blood vessel lumen of the donor organ.

The method may further include adding a crowding agent. The crowding agent may be selected from one or more of: a dextran; a dextran sulfate; a dextrin; a pullulan; a poly(ethylene glycol); a Ficoll™; a hyper-branched glycerol; and an inert protein. The method may include perfusing the donor organ with an organ perfusion or an organ preservation solution comprising an enzyme composition described herein.

The method may further include washing the donor organ to remove GalNAcDeacetylase, Galactosaminidase and/or the crowding agent.

The GalNAcDeacetylase and Galactosaminidase may be capable of cleaving A-antigen at or below 1 μg/ml. The GalNAcDeacetylase and Galactosaminidase may have A-antigen cleaving activity at a pH between about 6.5 and about 7.5. The GalNAcDeacetylase and Galactosaminidase may have A-antigen cleaving activity at a temperatures between 4° C. and 37° C.

The GalNAcDeacetylase and Galactosaminidase may be capable of cleaving A-antigen at or below 100 μg/ml. The GalNAcDeacetylase and Galactosaminidase may be capable of cleaving A-antigen at or below 90 μg/ml. The GalNAcDeacetylase and Galactosaminidase may be capable of cleaving A-antigen at or below 80 μg/ml. The GalNAcDeacetylase and Galactosaminidase may be capable of cleaving A-antigen at or below 70 μg/ml. The GalNAcDeacetylase and Galactosaminidase may be capable of cleaving A-antigen at or below 60 μg/ml. The GalNAcDeacetylase and Galactosaminidase may be capable of cleaving A-antigen at or below 50 μg/ml. The GalNAcDeacetylase and Galactosaminidase may be capable of cleaving A-antigen at or below 40 μg/ml. The GalNAcDeacetylase and Galactosaminidase may be capable of cleaving A-antigen at or below 30 μg/ml. The GalNAcDeacetylase and Galactosaminidase may be capable of cleaving A-antigen at or below 20 μg/ml. The GalNAcDeacetylase and Galactosaminidase may be capable of cleaving A-antigen at or below 1514/ml. The GalNAcDeacetylase and Galactosaminidase may be capable of cleaving A-antigen at or below 1.414/ml. The GalNAcDeacetylase and Galactosaminidase may be capable of cleaving A-antigen at or below 13 μg/ml. The GalNAcDeacetylase and Galactosaminidase may be capable of cleaving A-antigen at or below 12 μg/ml. The GalNAcDeacetylase and Galactosaminidase may be capable of cleaving A-antigen at or below 11 μg/ml. The GalNAcDeacetylase and Galactosaminidase may be capable of cleaving A-antigen at or below mpg/mi. The GalNAcDeacetylase and Galactosaminidase may be capable of cleaving A-antigen at or below 9 μg/ml. The GalNAcDeacetylase and Galactosaminidase may be capable of cleaving A-antigen at or below 8 μg/ml. The GalNAcDeacetylase and Galactosaminidase may be capable of cleaving A-antigen at or below 74/ml. The GalNAcDeacetylase and Galactosaminidase may be capable of cleaving A-antigen at or below 6 μg/ml. The GalNAcDeacetylase and Galactosaminidase may be capable of cleaving A-antigen at or below 5 μg/ml. The GalNAcDeacetylase and Galactosaminidase may be capable of cleaving A-antigen at or below 4 μg/ml. The GalNAcDeacetylase and Galactosaminidase may be capable of cleaving A-antigen at or below 3 μg/ml. The GalNAcDeacetylase and Galactosaminidase may be capable of cleaving A-antigen at or below 2 μg/ml. The GalNAcDeacetylase and Galactosaminidase may be capable of cleaving A-antigen at or below 1 μg/ml. The GalNAcDeacetylase and Galactosaminidase may be capable of cleaving A-antigen at or below 0.9 μg/ml. The GalNAcDeacetylase and Galactosaminidase may be capable of cleaving A-antigen at or below 0.8 μg/ml. The GalNAcDeacetylase and Galactosaminidase may be capable of cleaving A-antigen at or below 0.7 μg/ml. The GalNAcDeacetylase and Galactosaminidase may be capable of cleaving A-antigen at or below 0.6 μg/ml. The GalNAcDeacetylase and Galactosaminidase may be capable of cleaving A-antigen at or below 0.5 μg/ml. The GalNAcDeacetylase and Galactosaminidase may be capable of cleaving A-antigen at or below 0.4 μg/ml. The GalNAcDeacetylase and Galactosaminidase may be capable of cleaving A-antigen at or below 0.4 μg/ml. The GalNAcDeacetylase and Galactosaminidase may be capable of cleaving A-antigen at or below 0.2 μg/ml. The GalNAcDeacetylase and Galactosaminidase may be capable of cleaving A-antigen at or below 0.4 μg/ml. The GalNAcDeacetylase and Galactosaminidase may be capable of cleaving A-antigen at or below 0.09 μg/ml. The GalNAcDeacetylase and Galactosaminidase may be capable of cleaving A-antigen at or below 0.08 μg/ml. The GalNAcDeacetylase and Galactosaminidase may be capable of cleaving A-antigen at or below 0.07 μg/ml. The GalNAcDeacetylase and Galactosaminidase may be capable of cleaving A-antigen at or below 0.06 μg/ml. The GalNAcDeacetylase and Galactosaminidase may be capable of cleaving A-antigen at or below 0.05 μg/ml. The GalNAcDeacetylase and Galactosaminidase may be capable of cleaving A-antigen at or below 0.04 μg/ml. The GalNAcDeacetylase and Galactosaminidase may be capable of cleaving A-antigen at or below 0.03 μg/ml. The GalNAcDeacetylase and Galactosaminidase may be capable of cleaving A-antigen at or below 0.02 μg/ml. The GalNAcDeacetylase and Galactosaminidase may be capable of cleaving A-antigen at or below 0.01 μg/ml.

The GalNAcDeacetylase and Galactosaminidase may have A-antigen cleaving activity at a pH between about 6.5 and about 7.5. The GalNAcDeacetylase and Galactosaminidase may have A-antigen cleaving activity at a pH between about 6.0 and about 8.0. The GalNAcDeacetylase and Galactosaminidase may have A-antigen cleaving activity at a pH between about 6.8 and about 70.8. The GalNAcDeacetylase and Galactosaminidase may have A-antigen cleaving activity at a pH between about 6.9 and about 7.9. The GalNAcDeacetylase and Galactosaminidase may have A-antigen cleaving activity at a pH between about 6.4 and about 70.8.

The GalNAcDeacetylase and Galactosaminidase may have A-antigen cleaving activity at temperatures between 4° C. and 37° C. The GalNAcDeacetylase and Galactosaminidase may have A-antigen cleaving activity at temperatures between 3° C. and 38° C. The GalNAcDeacetylase and Galactosaminidase may have A-antigen cleaving activity temperatures between 4° C. and 40° C. The GalNAcDeacetylase and Galactosaminidase may have A-antigen cleaving activity at temperatures between 4° C. and 37° C. The GalNAcDeacetylase and Galactosaminidase may have A-antigen cleaving activity at a temperatures between 5° C. and 37° C.

In accordance with another embodiment, there is provided a purified enzyme including a Flavonifractor plautii GalNAcDeacetylase of SEQ ID NO.:2, SEQ ID NO.:4 or SEQ ID NO.:5.

In accordance with another embodiment, there is provided a purified enzyme including a Flavonifractor plautii Galactosaminidase of SEQ ID NO.:7, SEQ ID NO.:9 or SEQ ID NO.:10.

In accordance with another embodiment, there is provided a purified enzyme including a purified Clostridium tertium GalNAcDeacetylase and Galactosaminidase fusion protein of SEQ ID NO.:14.

In accordance with another embodiment, there is provided a vector including the nucleic acid as described herein and a heterologous nucleic acid sequence.

In accordance with another embodiment, the method may be carried out in vitro or ex vivo. As used herein ex vivo means that the method is carried out outside an organism. For example, ex vivo would encompass ex vivo lung perfusion (EVLP) and treatment of donated blood. As used herein, ex vivo refers to experimentation or measurements or treatments done in or on tissue or cells (for example, erythrocytes or a donor organ) from an organism in an external environment with minimal or some alterations of conditions from which the tissue or cells were under when in vivo.

### BRIEF DESCRIPTION OF THE DRAWINGS

FIG. 1 shows a schematic illustration of cell surface antigen carbohydrate structures terminating in α-1,3-linked-N-acetylgalactosamine (GalNAc) or galactose (Gal) for A-type, H-type and B-type, wherein the triangles mark the cleavage points for the α-Nacetyl-galactosaminidase EmGH109 and α-galactosidase BfGal110.

FIG. 2 shows the deacetylation enzymatic pathway of A antigen cleavage, whereby Flavonifractor plautii (Fp) GalNAcDeacetylase cleaves the acetyl group from the terminal α-N-acetyl-galactosamine of the A antigen (−₄2 m/z) and the galactosaminide intermediate is then cleaved by the Flavonifractor plautii (Fp) Galactosaminidase (−161_{m/z}), with corresponding mass-spectrometry (MS) analysis.

FIG. 3 shows FACS analysis of A⁺ RBCs treated with different concentrations of EmGH109 or Flavonifractor plautii GalNAcDeacetylase (FpGalNAcDeacetylase) plus Flavonifractor plautii Galactosaminidase (FpGalactosaminidase) or for 1 h at 37° C., wherein for visualization anti-H-antibody (plus secondary FITC-labelled) and APC labelled anti-A-antibody were used, where the area for the appearance of H antigens are in the upper left hand box. Rows A-D compare EmGH109 and FpGalNAcDeAc+FpGalNase at 5 μg/ml (A); 10 μg/ml (B); 50 μg/ml (C); and 50 μg/ml+dextran 40k(D).

FIG. 4 shows a comparison of EmGH109 with FpGalNAcDeAc+FpGalNase at various enzyme concentrations with (▪) and without(♦) dextran at various temperatures (i.e. 4° C., room temperature (RT) and 37° C.).

FIG. 5 shows HPAE-PAD analysis of A+B+ and O+erythrocyte cleavage products and a comparison of full length Flavonifractor plautii GalNAcDeacetylase (FpGalNAcDeAc)+Flavonifractor plautii Galactosaminidase (FpGalNase) enzymes with truncated FpGalNAcDeAc+FpGalNase enzymes on A+erythrocytes.

FIG. 6 shows pH profiles for each of (A) FpGalNAcDeacetylase and (B) FpGalactosaminidase.

FIG. 7 shows conversion of A antigen to H antigen on A RBCs as analysed via FACS, for (A) A+RBC control, (B) Flavonifractor plautii GalNAcDeacetylase (FpGalNAcDeAc)+Flavonifractor plautii Galactosaminidase (FpGalNase) (10 ug/mL), (C) FpGalNAcDeAc+Clostridium tertium (Ct) Ct5757_GalNase (10 ug/mL) and (D) FpGalNAcDeAc+Robinsoniella peoriensis (Rp) Galactosaminidase (Rp1021) GalNase (10 ug/mL).

FIG. 8 shows dose escalation effects of enzymes on A antigen removal from type A human red cells in different perfusion solutions (i.e. PBS, Steen™, and Perfadex™)

FIG. 9 shows dose escalation effects of the enzymes on type A human arteries in STEEN solution, wherein the percent of type A antigen is quantified from immunohistochemical analysis of biopsies taken from untreated (control), treated (treatment) type A arteries and type O arteries as a negative control.

FIG. 10 shows the effects of 1-hour enzymatic treatments on ex vivo perfused human donor lungs was tested, where immunohistochemical staining of biopsied human donor lungs compared pretreatment images with post-treatment images of the right upper dependent (RUD), right upper non-dependent (RUND), right middle non-dependent (RMND), right middle dependent (RMD), right lower non-dependent (RLND), and right lower dependent (RLD) areas of the lung, the blood type A antigens are absent in the blood vessels.

FIG. 11 shows the effects of 3-hour enzymatic treatments on ex vivo perfused human donor lungs was tested, where immunohistochemical staining of biopsied human donor lungs compared pretreatment images with post-treatment images of the right upper dependent (RUD), right upper non-dependent (RUND), right middle non-dependent (RMND), right middle dependent (RMD), right lower non-dependent (RLND), and right lower dependent (RLD) areas of the lung, the blood type A antigens are absent in the blood vessels.

### DETAILED DESCRIPTION

The following detailed description will be better understood when read in conjunction with the appended figures. For the purpose of illustrating the invention, the figures demonstrate embodiments of the present invention. However, the invention is not limited to the precise arrangements, examples, and instrumentalities shown.

Any terms not directly defined herein shall be understood to have the meanings commonly associated with them as understood within the art of the invention.

An “immobilized enzyme” as used herein is an enzyme attached to surface, which may be an inert, insoluble material. Immobilization of enzymes can provide increased resistance to changes in conditions such as pH, temperature etc. and assist in their removal following use and for enzyme re-use.

Immobilization of an enzyme may be accomplished by various ways (for example, affinity-tag binding, surface adsorption on glass, resin, alginate beads or matrix, bead, fiber or microsphere entrapment, cross-linking to a surface or other enzymes and covalent binding to a surface).

As used herein “affinity-tag binding” refers to the immobilization of enzymes to a surface (for example, a porous material, using non-covalent or covalent protein tags). Affinity-tag binding has been used for protein purification and has more recently been used for biocatalysis applications by EziG™ (ENGINZYME AB™, Sweden—for example, PCT/US1992/010113; and PCT/SE2015/050108). Alternative systems are known in the art for attaching active enzymes to a surface (see for example, U.S. Pat. Nos. 4,088,538; 4,141,857; 4,206,259; 4,218,363; 4,229,536; 4,239,854; 4,619,897; 4,748,121; 4,749,653; 4,897,352; 4,954,444; 4,978,619; 5,154,808; 5,914,367; 5,962,279; 6,030,933; 6,291,582; 6,254,645; 10,016,490; and 10,041,055)

Protein tags are peptide sequences genetically grafted onto a recombinant protein, are often removable by chemical agents or by enzymatic means and are attached to proteins for various purposes. The protein tags set out in TABLE A are intended to be examples and are not intended to be limiting in any way. One type of protein tag is an affinity tag, which are added to proteins or peptide sequences so that they can be purified from a crude biological source using an affinity technique (for example, from expression system organisms) or to facilitate immobilization of the “tagged” protein to a surface. Some examples of affinity tags include chitin binding domain (CBD), maltose binding protein (MBP), Strep-tag, glutathione-S-transferase (GST) and the Polyhistidine (His-tag), which binds to metal matrices. Another type of protein tag is a epitope tag (for example, include V5-tag, Myc-tag, HA-tag, Spot-tag and NE-tag), which are short peptide sequences chosen for the ease of producing high-affinity antibodies and are often derived from viral gene sequences to improve immunoreactivity. Epitope tags are particularly useful for western blotting, immunofluorescence and immunoprecipitation experiments, although they also find use in purification and immobilization of proteins to a surface. Yet another type of protein tag is a chromatography tag (for example, polyanionic amino acids, such as FLAG-tag), which may be used to alter chromatographic properties of the protein to assist with separation and purification or immobilization. Yet further protein tags are solubilization tags (for example, Maltose-binding protein (MBP), Glutathione S-transferase (GST), thioredoxin (TRX) and poly(NANP)) and fluorescence tags (for example, Green fluorescent protein (GFP)). Protein tags may allow specific enzymatic modification, chemical modifications or to connect proteins to other components. However, depending on the type or number of tags added to a protein sequence the native function of the protein, in this case the enzymatic function, may be compromised by the tag. Accordingly, the protein tag would need to be selected to ensure that the activity of the enzyme is not compromised or alternatively, the protein tag may be cleaved from the protein before use.

TABLE A
Exemplary Protein Tags

| | | SEQ ID | |
|---|---|---|---|
| Tag Name | Length (sequence) | NO: | Position |
| Albumin-binding | 137 | | N-term or C-term |
| protein (ABP) | | | |
| Alkaline Phosphatase | 444 | | Primary amines (lysine side |
| (AP) | | | chain epsilon-amines and N- |
| | | | terminal a-amines) |
| AU1 epitope | 6 (DTYRYI) | 59 | N-term or C-term |
| AU5 epitope | 6 (TDFYLK) | 60 | N-term or C-term |
| AviTag | 15 | 61 | |
| | (GLNDIFEAQKIEWHE) | | |
| Bacteriophage T7 | 11 (MASMTGGQQMG) | 62 | N-term or internal |
| epitope (T7-tag) | | | |
| Bacteriophage V5 | 14 (GKPIPNPLLGLDST) | 63 | C-term |
| epitope (V5-tag) | | | |
| Biotin-carboxy carrier | 100 | | N-term or C-term |
| protein (BCCP) | | | |
| Bluetongue virus tag | 6 (QYPALT) | 64 | N-term or C-term |
| (B-tag) | | | |
| peptide tag binds | 4 (EPEA) | 65 | |
| single-domain | | | |
| camelid antibody (C- | | | |
| tag) | | | |
| Calmodulin binding | 26 | 66 | N-term or C-term |
| peptide (CBP or | (KRRWKKNFIAVSAANR | | |
| Calmodulin-tag) | FKKISSSGAL) | | |
| Chloramphenicol | 218 | | N-term |
| Acetyl Transferase | | | |
| (CAT) | | | |
| Cellulose binding | 27-189 | | N-term, C-term, or internal |
| domain (CBP) | | | (domain-dependent) |
| Chitin binding domain | 51 | | N-term or C-term |
| (CBD) | | | |
| Choline-binding | 145 | | N-term |
| domain (CBD) | | | |
| Dihydrofolate | 227 | | N-term or C-term |
| reductase (DHFR) | | | |
| DogTag | 23 | | 67 |
| | (DIPATYEFTDGKHYITN | | |
| | EPIPPK) | | |
| E2 epitope | 10 (SSTSSDFRDR) | 68 | N-term, C-term or internal |
| E-tag | 13 (GAPVPYPDPLEPR) | 69 | |
| FLAG epitope (FLAG- | 8 (DYKDDDK) | 70 | N-term or C-term |
| tag) | | | |
| Galactose-binding | 509 | | N-term or C-term |
| protein (GBP) | | | |
| Green fluorescent | 220 | | N-term or C-term |
| protein (GFP) | | | |
| Glu-Glu (EE-tag) | 6 (EYMPME or | 71 | N-term, or C-term, or internal |
| | EFMPME) | 72 | |
| Glutathione S- | 211 | | N-term or C-term |
| transferase (GST) | | | |
| Human influenza | 31 | | N-term, C-term or internal |
| hemagglutinin (HA) | | | |
| hemagglutinin | 9 (YPYDVPDYA) | 73 | |
| recognized by an | | | |
| antibody (HA-tag) | | | |
| HaloTag® | 312 | | N-term or C-term |
| Histidine affinity tag | 19 | 74 | N-term or C-term |
| (HAT) | (KDHLIHNVHKEFHAHA | | |
| | HNK) | | |
| Histidine and | 6 (HQHQHQ) | 75 | |
| glutamine are | | | |
| alternating peptide | | | |
| tags (HQ-tag) | | | |
| Horseradish | 400 | | Primary amines (lysine side |
| Peroxidase (HRP) | | | chain epsilon-amines and N- |
| | | | terminal a-amines) |
| Histidine and | 12 (HNHNHNHNHNHN) | 76 | |
| asparagine alternating | | | |
| peptide tags (HN-tag) | | | |
| HSV epitope | 11 (QPELAPED) | 77 | C-term |
| Isopeptag peptide | 16 | 78 | |
| binding covalently to | (TDKDMTITFTNKKDAE) | | |
| pilin-C protein | | | |
| (Isopep-tag) | | | |
| Ketosteroid isomerase | 125 | | N-term |
| (KSI) | | | |
| KT3 epitope | 11 (KPPTPPPEPET) | 79 | N-term or C-term |
| LacZ | 1024 | | N-term or C-term |
| Luciferase | 551 | | N-term |
| Maltose-binding | 396 | | N-term or C-term |
| protein (MBP) | | | |
| Myc epitope | 11 (CEQKLISEEDL) | 80 | N-term, C-term or internal |
| c-myc peptide epitope | 10 (EQKLISEEDL) | 81 | |
| (Myc-tag) | | | |
| Synthetic peptide | 18 | 82 | |
| (NE-tag) | (TKENPRSNQEESYDDN | | |
| | ES) | | |
| NusA | 495 | | N-term or C-term |
| PDZ domain | 80-90 | | N-term, C-term, or internal |
| PDZ ligand | 5-7 (varies) | | C-term |
| Polyarginine (Arg-tag) | 5-6 (usually 5; RRRRR) | 83 | C-term |
| Polyaspartate (Asp- | 5-16 (DDDDD) | 84 | C-term |
| tag) | | | |
| Polycysteine (Cys-tag) | 4 (CCCC) | 85 | N-term |
| polyglutamate tag | 6 (EEEEEE) | 86 | |
| (Glu-tag) | | | |
| Polyhistidine (His- | 2-10 (usually 6; | 87 | N-term or C-term |
| tag) | HHHHHH) | | |
| Polyphenylalanine | 11 (FFFFFFFFFFF) | 88 | N-term |
| (Phe-tag) | | | |
| Profinity eXact | 75 | | N-term |
| Protein C | 12 | | N-term or C-term |
| rhodopsin (bovine) | 9 (TETSQVAPA) | 89 | |
| intracellular C- | | | |
| terminus peptide | | | |
| (Rho1D4-tag) | | | |
| S1-tag | 9 (NANNPDWDF) | 90 | N-term or C-term |
| Ribonuclease A | 15 | 91 | N-term, C-term or internal |
| derived (S-tag) | (KETAAAKFERQHMDS) | | |
| Softag 1, for | 13 (SLAELLNAGLGGS) | 92 | |
| mammalian | | | |
| expression | | | |
| Softag 3, for | 8 (TQDPSRVG) | 93 | |
| prokaryotic | | | |
| expression | | | |
| SpyTag peptide binds | 13 (AHIVMVDAYKPTK) | 94 | |
| covalently to | | | |
| SpyCatcher protein | | | |
| (Spy-tag) | | | |
| Streptavidin binding | 38 | 95 | C-term |
| tag (SBP-tag or SBP) | (MDEKTTGWRGGHVVE | | |
| GLAGELEQLRARLEHHP | | | |
| QGQREP) | | | |
| Staphylococcal | 280 | | N-term |
| protein A (Protein A) | | | |
| Staphylococcal | 280 | | N-term or C-term |
| protein G (Protein G) | | | |
| Strep-tag | 8-9 (WSHPQFEK) or | 96 | N-term or C-term |
| | (AWAHPQPGG) | 97 | |
| Streptavadin (Strep- | 159 | | N-term or C-term |
| tag) | | | |
| Strep-tag peptide | 8 (WSHPQFEK) | 98 | |
| binding streptavidin | | | |
| or streptactin (Strep- | | | |
| tag II) | | | |
| SdyTag (Sdy-tag) | 13 (DPIVMIDNDKPIT) | 99 | |
| Small Ubiquitin-like | 100 | | N-term |
| Modifier (SUMO) | | | |
| SnoopTag | 12 (KLGDIEFIKVNK) | 100 | |
| SnoopTagJr | 12 (KLGSIEFIKVNK) | 101 | |
| Spot-tag | 12 (PDRVRAVSHWSS) | 102 | |
| Tandem Affinity | Variable | | N-term or C-term |
| Purification (TAP) | | | |
| T7 epitope | 260 | | N-term |
| Tetracysteine tag | 6 (CCPGCC) | 103 | |
| (TC-tag) | | | |
| Thioredoxin (Trx) | 109 | | N-term or C-term |
| TrpE | 25-336 | | N-term or C-term |
| Ty-tag | 10 (EVHTNQDPLD) | 104 | |
| Ubiquitin | 76 | | N-term |
| Universal | 6 (HFIPHH) | 105 | N-term, C-term or internal |
| VSV-G or VSV-tag | 11 (YTDIEMNRLGK) | 106 | C-term |
| V5-tag | 14 (GKPIPNPLLGLDST) | 107 | |
| Xpress tag | 8 (DLYDDDDK) | 108 | |

The use of a protein tag is exemplified in the current application through the use of Polyhistidine protein tag (His-tag) as shown in SEQ ID NOs: 5, 10, 15, 17, 19, 21, 23, 25, 27, 29 and 31, but a person of skill in the art would readily appreciate that any number of other protein tags may be used to purify the enzymes and/or be used to attach the enzymes to a surface as described herein, depending on the purification method used and/or the surface the enzymes are attached to. Such protein tags may be selected from any one or more of the protein tags listed in TABLE A, but other such protein tags are known in the art.

Furthermore, the use of one or more cleavage sites (for example, the thrombin cleavage site as used in SEQ ID NOs: 15, 17, 19, 21, 23, 25, 27, 29 and 31) may be employed to release the protein tag from the enzyme or to otherwise cleave the enzyme. A cleavage site may be used for the removal of the N-terminal methionine, signal peptide, and/or the conversion of an inactive or non-functional protein to an active one (i.e. zymogens or proenzymes). Alternatively, a cleavage site may be used to separate two or more enzymes that were expressed in the same reading frame. Examples of enzymes that are capable of cleaving proteins or peptides and which would have sequence specific cleavage sites may be selected from one or more of the following: Arg-C proteinase; Asp-N endopeptidase; Asp-N endopeptidase+N-terminal Glu BNPS-Skatole; Caspase 1; Caspase 2; Caspase 3; Caspase 4; Caspase 5 Caspase 6; Caspase 7; Caspase 8; Caspase 9; Caspase 10; Chymotrypsin-high specificity (C-term to [FYW], not before P); Chymotrypsin-low specificity (C-term to [FYWML], not before P); Clostripain (Clostridiopeptidase B); CNBr; Enterokinase; Factor Xa; Formic acid; Glutamyl endopeptidase; GranzymeB; Hydroxylamine; Iodosobenzoic acid; LysC; LysN; NTCB (2-nitro-5-thiocyanobenzoic acid); Neutrophil elastase; Pepsin (pH1.3); Pepsin (pH>2); Proline-endopeptidase; Proteinase K; Staphylococcal peptidase I; Tobacco etch virus protease; Thermolysin; Thrombin; and Trypsin.

A person of skill in the art would appreciate that the combination of an active Galactosaminidase enzyme and an active GalNAcDeacetylase enzyme, as described herein, capable of efficiently cleaving A-antigen is of importance and that person of skill would also appreciate that the addition of one or more cleavage sites and/or one or more protein tags is optional and that such modifications may be selected based on the particular expression system, purification system and possible surface attachment strategy. Furthermore, other modifications to the Galactosaminidase and the GalNAcDeacetylase sequences are possible, provided that the activity in cleaving A-antigens is not significantly impaired. Additionally, modifications to the Galactosaminidase and the GalNAcDeacetylase enzymes is possible, provided that the A-antigen cleavage activity is not significantly impaired. The modifications to the Galactosaminidase and the GalNAcDeacetylase sequences may be a deletion, an insertion and/or a substitution. The substitution may be a conservative substitution or a neutral substitution. For example, the Galactosaminidase and the GalNAcDeacetylase sequences may share 90% or more sequence identity with the mature enzymes is possible. For example, the Galactosaminidase and the GalNAcDeacetylase sequences may share 85% or more sequence identity with the mature enzymes is possible. For example, the Galactosaminidase and the GalNAcDeacetylase sequences may share 75% or more sequence identity with the mature enzymes is possible. Alternatively, the Galactosaminidase and the GalNAcDeacetylase sequences may have modifications to 5, 10, 13, 15, 20 or up to 25%, of the amino acids.

As used herein “adsorption on glass, alginate beads or matrix” refers to the attached of an enzyme to the outside of an inert material. Generally, this type of immobilization does not result from a chemical reaction and the active site of the immobilized enzyme can be blocked by the surface to which it has absorbed, which may reduce the activity of the enzyme being absorbed.

As used herein “entrapment” refers to the trapping of an enzyme within an insoluble beads or microspheres. However, entrapment may hinder the arrival of the substrate, and the exit of products. One example, is the use of as calcium alginate beads, which may be produced by reacting a mixture of sodium alginate solution and enzyme solution with calcium chloride.

As used herein “cross-linkage” refers to the covalent bonding of enzymes to each other to create a matrix consisting of almost only enzyme. When a cross-linkage enzyme reaction is designed, the binding site ideally does not cover the enzyme's active site so that the activity of the enzyme is only affected by immobility and not by blockage of the enzyme's active site. Nevertheless, spacer molecules like poly(ethylene glycol) may be used to reduce the steric hindrance by the substrate.

As used herein “covalent bonding” refers to the bonding of an enzyme to an insoluble support or surface (for example, a silica gel) via a covalent bond. Due to the strength of the covalent bonds between the enzymes and the support or surface, there is much less likelihood of enzymes detaching from the support or surface.

As used herein “crowding agent” refers to any polymer or protein that facilitates macromolecular crowding by concentrating enzyme on the cell surface to improve activity of the enzyme. A crowding agent may for example be a dextran, a dextran sulfate, a dextrin, a pullulans, a poly(ethylene glycol), a Ficoll™, a hyper-branched glycerol and an inert protein. (Kuznetsova, I. M et al. Int J Mol Sci. (2014) “What Macromolecular Crowding Can Do to a Protein” 15(12): 23090-23140).

As used herein “dextran” refers to a polysaccharide with molecular weights ≥1,000 Daltons and having a linear backbone of α-linked d-glucopyranosyl repeating units. Dextrans may divided into 3 structural classes (i.e. classes 1-3) based on the pyranose ring structure, which contains five carbon atoms and one oxygen atom. Class 1 dextrans contain the α(1→6)-linked d-glucopyranosyl backbone modified with small side chains of d-glucose branches with α(1→2), α(1→3), and α(1→4)-linkage. The class 1 dextrans vary in their molecular weight, spatial arrangement, type and degree of branching, and length of branch chains, 3-5 depending on the microbial producing strains and cultivation conditions. Isomaltose and isomaltotriose are oligosaccharides with the class 1 dextran backbone structure. Class 2 dextrans (alternans) contain a backbone structure of alternating α(1→3) and α(1→6)-linked d-glucopyranosyl units with α(1→3)-linked branches. Class 3 dextrans (mutans) have a backbone structure of consecutive α(1→3)-linked d-glucopyranosyl units with α(1→6)-linked branches.

As used herein, “pullulans” are structural polysaccharides primarily produced from starch by the fungus Aureobasidium pullulans and are composed of repeating α(1→6)-linked maltotriose (D-glucopyranosyl-α(1→4)-D-glucopyranosyl-α(1→4)-D-glucose) units with the inclusion of occasional maltotetraose units.

As used herein, “dextrin” refers to D-glucopyranosyl units with a shorter chain lengths than dextran, which start with a single α(1→6) bond, but continue linearly with α(1→4)-linked D-glucopyranosyl units.

As used herein, “Ficoll™” is a neutral, highly branched, high-mass, hydrophilic polysaccharide, which dissolves readily in aqueous solutions.

As used herein, “perfusion” or “perfusing” refers to permeating an organ with a fluid by circulating the fluid through blood vessels.

An important goal in organ preservation is to increase the number of available transplantable organs. Typically, organs were kept in cold storage, but this has potential diffusional limitations, and thus cold perfusion systems have been developed. Furthermore, near-normothermic systems are also being used to enhance the functional preservation of solid organs including livers, lungs, hearts and kidneys. A number of buffered extracellular solutions are used as perfusion solutions or preservation solutions. A number of buffered extracellular solutions are known. For example, Steen™, Perfadex™, Perfadex Plus™, EuroCollins, Histidine-Tryptophan-Ketoglutarate (HTK) solution, University of Wisconsin Solution (UW), Celsior solution, Kidney Perfusion Solution (KPS-1), Kyoto University Solution, IGL-1 Solution, and Citrate solution (Guibert, E. E. et al. (2011)). Many of these are commercially available and variations to these solutions would be apparent to persons of skill in the art.

Various alternative embodiments and examples are described herein. These embodiments and examples are illustrative and should not be construed as limiting the scope of the invention.

### Materials and Methods

Chemicals and commercial enzymes used in this study were purchased from Sigma-Aldrich™ unless otherwise stated. Monosaccharide methylumbelliferyl glycosides were a generous gift from Dr. Hongming Chen and the A-antigen subtype1_{penta-MU} was a generous gift from Dr. David Kwan (Kwan et al. 2015).

Human Feces Metagenomic Library

For the generation of the human metagenomics fosmid library human fresh fecal samples were collected from a healthy Asian male volunteer having blood group AB+. The direct DNA extraction and fosmid library creation were performed according to the procedure described in the MoE Protocol (Armstrong et al. 2017).

Fosmid Library Screening

51×384-well AB⁺ Blood Fosmid library plates were thawed at room temperature and replicated into 384-well plates containing 50 μl screening LB-media (12.5 μg/mL chloramphenicol, 25 μg/mL kanamycin, 100 μg/mL arabinose, 0.2% (v/v) maltose, 10 mM MgSO₄). Plates were incubated at 37° C. for 18 hours in a sealed container containing a reservoir of water to prevent excessive evaporation. 45 μl of the reaction mixture (100 mM NaH2PO4, pH 7.4, 2% (v/v) Triton-X 100, 100 μM GalNAc-α-MU, 100 μM Gal-α-MU) were added onto grown screening plates using the QFill™ instrument [Genetix™]. The plates were then incubated at 37° C. in a sealed container for 24 h, and the fluorescence (Ex: 365 nm Em: 435 nm, sweep-mode, gain 80) of each plate was measured at hours 1, 2, 4, 8 and 24 via a Synergy H1 plate reader [BioTek™]. For all wells a Z-score was calculated, which is given by the formula: Z-score=(Fluorescence-median value)/Standard Deviation.

All positive hits above a certain threshold, were re-arrayed in a new 384-well plate, designated the “simple substrate hit” plate and stored at −70° C. Two screening plates were replicated from the “simple substrate hit” plate and re-screened for either GalNAc-α-MU or Gal-α-MU activity to verify and deconvolute the previously detected activity.

To determine which of the hits can cleave A-antigen or B-antigen structures, their activity on 50 μM A antigen subtype 1tetra-MU or 50 μM B antigen subtype_{1 tetra}-MU was determined using a coupled enzyme assay. A version of this coupled assay was described previously by Kwan (Kwan et al. 2015). Our assay was modified to also detect cleavage of the subtype 1A antigen, by use of BgaC (Jeong 2009) instead of BgaA (Singh 2014) as coupling enzyme. Potential α-N-acetylgalactosaminidases or α-galactosidases would cleave the terminal sugar, releasing the H antigen subtype Iₜᵣᵢ-MU. Subsequently an α-fucosidase (AfcA (Katayarna 2004)), β-galactosidase (BgaC (Jeong 2009)) and β-hexosaminidase (SpHex (Williams 2002)) will cleave the residual sugars in exo-fashion, until 4-methylumbelliferyl alcohol is released; detectable as increase of the fluorescence. To achieve this, 50 μg/mL of each enzyme was added to the reaction mixture. All positive hits above a certain threshold were re-screened in triplicate and a host cell strain containing a vector lacking any insert was used as a negative control. All verified hits were stored separately at −70° C. in LB-media (12.5 μg/mL chloramphenicol, 25 μg/mL kanamycin, 15% (v/v) glycerol, 0.2% (v/v) maltose, 10 mM MgSO₄).

Fosmid Hit Sequencing

To isolate the fosmid DNA for sequencing, the positive hit fosmid glycerol stocks were used to inoculate 5 mL of TB media (12.5 μg/mL chloramphenicol, 25 μg/mL kanamycin, 100 μg/mL arabinose, 0.2% (v/v) maltose, 10 mM MgSO₄), incubated overnight at 37° C. 220 rpm. Fosmid isolation was performed using the GeneJet™ plasmid miniprep kit (Thermo Fisher™). The isolated fosmids were purified from contaminating linear E. coli. DNA using Plasmid-Safe™ ATP-Dependent DNase (Epicentre™), followed by another round of purification with a GeneJet™ PCR purification kit (Thermo Fisher™). Concentration was calculated with a Quant-iT™ dsDNA HS Assay Kit (Invitrogen™) on a Qbit™ fluorimeter (ThermoFisher™). Expected DNA size was validated with a 1% agarose gel. For full fosmid sequencing, 2 ng of each fosmid was sent to the UBC Sequencing Centre (Vancouver, BC, Canada). Each fosmid was individually barcoded and sequenced using an Illumina MiSeg™ system.

All Illumina MiSeg™ raw sequence data were trimmed and assembled using a python script available on GitHub™ at https://github.com/hallamlab/FabFos. Briefly, Trimmomatic was used to remove adapters and low-quality sequences from the reads (Bolger 2014). These reads were screened for vector and host sequences using BWA (Li 2013) and then filtered using Samtools™ and a bam2fastq script to remove contaminants. These high-quality and purified reads were assembled by MEGAHIT with k-mer values ranging between 71 and 241, increasing by increments of 10 (Li 2015). Since these libraries often had in excess of 20,000 times coverage and to prevent the accumulation of sequencing errors interfering with proper sequence assembly, the minimum k-mer multiplicity was calculated by 1% of the estimated coverage of a fosmid. Outside of the python script assemblies, which yielded more than one contig were then scaffolded using minimus2 (Treangen 2011). Parameterized commands can be found in both documentation on the GitHub™ page and in the python script itself.

Fosmid ORF Prediction and Hit Validation

Fosmid ORFs were identified using the metagenomic version of Prodigal™ (Hyatt 2010) and compared to the CAZy™ database using BLASTP™ as part of the MetaPathways™ v2.5 software package (Konwar 2015). MetaPathways™ parameters: length >60, BLAST score >20, blast score ratio >0.4, E_{Value}<1×10-6.

All predicted ORFs with annotations to members of a GH or CBM family (with known or suspected α-galactosidase and/or α-N-acetylgalactosaminidase activities) were cloned into pET16b plasmid using the Golden Gate™ cloning strategy (Engler 2008), the primer sequences are set out in TABLE B. The proteins were expressed in BL21(DE3), cultured in 10 mL ZY5052 auto induction media (Studier 2005) for 20 h at 37° C., 220 rpm. Cells were harvested by centrifugation (40 00×g, 4° C., 10 min) and resuspended in 1 mL lysis buffer (100 mM NaH₂PO₄, pH 7.4, 2% (v/v) Triton-X™ 100, 1× Protease Inhibitor EDTA-free [Pierce™]). A coupled assay (Kwan 2015) was performed with 50 μl crude cell lysate from the candidates mixed with 50 μ1 assay buffer (100 mM NaH₂PO₄, pH 7.4, 50 μg/mL SpHex, 50 μg/mL AfcA, 50 μg/mL BgaC, 100 μM A antigen subtype 1ₜₑₜᵣₐ-MU or 100 μM B antigen subtype 1tetra-MU) and incubated at 37° C. All reactions were performed as triplicates in a black 96-well plate. Fluorescence (365/435 nm) was monitored continuously for 4 hours using a Synergy™ H1 plate reader [BioTek™]. Assays from crude extracts showing cleavage activity for A or B antigen were repeated, this time without the coupled enzymes, and the reaction product was isolated via an HF Bond Elut C18 column and analysed with LC-MS and/or TLC. TLC was performed using TLC Silica Gel 60 F254 TLC plates [EMD Millipore Corp.™, Billerica, Mass., USA].

TABLE B
Primer Sequences

| | | SEQ ID |
|---|---|---|
| Primer | Sequence | NO: |
| FpGalNAcDeAc_withoutSignalP_fw | ATGGTCTCGCCATGCAGACTCCAGCGAGTCCG | 38 |
| FpGalNAcDeAc_D1min_rv | ATGGTCTCGATTCTTACGTCGTGTAGCCGGGGTC | 39 |
| FpGalNAcDeAc_D1ext_rv | ATGGTCTCGATTCTTAATCACTGGAGGTATATTTCACGACC | 40 |
| FpGalNAcDeAc_D1+2_rv | ATGGTCTCGATTCTTACGCAGGCTCGATTGGACCATAC | 41 |
| FpGalNAcDeAc_D2ext_fw | ATGGTCTCGCCATGATGTGGCGACGGTGGATGAG | 42 |
| FpGalNAcDeAc_rv | ATGGTCTCGATTCTTATTCTCCCACATACGAAAAATAGTCG | 43 |
| FpGalNase_withoutSignalP_fw | ATGGTCTCGCCATCGTGGTAAAAAGTTCATATCACTCAC | 44 |
| FpGalNase_truncA_rv | ATGGTCTCGATTCTTATGCGTTAGTGGTATAAGTCAAATAGTC | 45 |
| FpGalNase_rv | ATGGTCTCGATTCTTATTCCGAAATTTCCACCGCTTTAAC | 46 |
| Ct5757_fw | ATGGTCTCGccatTATAATTTAATTGATAATATTAGTGTTGAAAAATTAG | 47 |
| Ct5757_rv | ATGGTCTCGattcTTATTGTGTTAAACCCTCAATAAAC | 48 |
| Ct5757_GalNase_rv | ATGGTCTCGattcTTAATGAGTACTTTGATTTAATCCATCATAAG | 49 |
| Ct5757_DeAcase_fw | ATGGTCTCGccatTCAGGGCAATATTGGTTAGTTTTC | 50 |
| Rp31021_fw | ATGGTCTCGccatGGGAACGGATTAGAGGTGAAAG | 51 |
| Rp31021_rv | ATGGTCTCGattcTCATAATACCATTTTGTATTTCTTTATATTGG | 52 |
| R18755_fw | ATGGTCTCGccatGAAGAAACCGATTTGCTTGTAAAC | 53 |
| R18755_rv | ATGGTCTCGattcTTAGCGTTCCAATATTTTCATAAATTCAG | 54 |
| Rp3671_fw | ATGGTCTCGccatTCACCATTGAGCGCTGCGG | 55 |
| Rp3671_rv | ATGGTCTCGattcTTATGACTTTGTTTTAACATTTACAGACTTG | 56 |
| Rp33672_fw | ATGGTCTCGccatGCTGAGACTGCAACAGAAGAAAATG | 57 |
| Rp33672_rv | ATGGTCTCGattcTTATTTCTGAATTTTTGCCTTGCCAG | 58 |

HPAE-PAD Assay

The analysis of the enzymatic release of galactosamine was carried out on an HPAE-PAD (Dionex™) HPLC system. Cleavage activity of the different proteins was tested on the following substrates: 7.5 μg/μL mucin from porcine stomach Type II in 100 mM NaH₂PO₄ pH 7.4; 5 mM A antigen subtype 1ₚₑₙₜₐ-MU in 100 mM NaH2PO4 pH 7.4 and RBCs (50% hematocrit) from A+, B+ and O-Type Donors in 1×PBS pH 7.4. Samples containing 10 μg/mL enzyme were incubated for two hours at 37° C. then stored at −80° C. for further analysis. Small aliquots of the reaction (10 μl) were diluted in H₂O (100 μl) and submitted to analysis on the HPAE-PAD instrument. Separation was performed on a CarboPAC PA200™ (150 mm) column with guard column, and detection was achieved using a disposable gold on polytetrafluoroethylene (PTFE) electrode and a four-potential waveform. The separation conditions were as follows: 100 mM sodium hydroxide and a sodium acetate gradient from 70 to 300 mM over the first 10 min of the separation. The eluent was held at the final gradient conditions for 1 mM and then returned to the starting conditions over the next minute. The flow rate was 1.0 ml/min and an injection was made every 27 min. A standard of the free sugars GalNAc, Gal and GalN (10 μM) was also applied to HPAE-PAD to determine the peak elution time for reference.

Kinetic Assays

All kinetic assays utilizing 4-methylumbelliferone as leaving group were performed through measurement of fluorescence. To avoid measurement errors based on the inner filter effect (Palmier 2007) standard curves were used to validate the linear range of the fluorophore.

FpGalactosaminidase

Michaelis-Menten parameter was determined for GalN antigen subtype 1ₚₑₙₜₐ-MU and A antigen subtype 1ₚₑₙₜₐ-MU in 100 mM NaH₂PO₄, pH 7.4 at 37° C. Reaction was performed in too with 3.4 nM FpGalactosaminidase (5.31 nM FpGalNase_truncA) and 0.1 mg/mL SpHex, AfcA, 0.2 mg/mL BgaC and varying concentrations of substrate (5 μM-2 mM). The reactions were run as a series of four with controls (no FpGalactosaminidase) as duplicates. The fluorescence signal (365/435 nm) resulting from MU release by hydrolysis was monitored by Synergy H1™ plate reader [BioTek™] and converted to concentration using MU standard concentration curves determined under identical reaction conditions. Initial rates (μM/s) were determined and plotted in Grafit 7.0™ to determine the kinetic parameters.

k_{cat}/K_{M} parameter was determined for GalN antigen subtype 1/2/4tetra-MU and B antigen subtype 1ₜₑₜᵣₐ-MU at pH7.4 and 37° C. Reactions (total volume of 100 μL) were performed in black 96-plate wells and as coupled assays in 100 mM NaH₂PO₄ (pH 7.4) with 8.63 nM FpGalactosaminidase, 0.1 mg/mL SpHex, BgaC (BgaA for Subtype 2), AfcA, varying concentrations of substrate (25 μM, 20 μM, 15 μM, 10 μM, 7.5 μM, 5 μM). The reactions were run as a series of four with controls (no FpGalactosaminidase) as duplicates. The fluorescence signal (365/435 nm) resulting from MU release by hydrolysis was monitored by Synergy H1™ plate reader [BioTek™] and converted to concentration using MU standard concentration curves determined under identical reaction conditions. Initial rates (μM/s) were determined and plotted in Grafit 7.0™ to determine the k_{cat}/K_{M} (s⁻¹*mM⁻¹) parameters.

Michaelis-Menten parameters were determined for GalN-α-pNP in in clear 96-plate at 37° C. with 8630.2 nM FpGalactosaminidase (in 100 mM NaH₂PO₄, pH70.4) or 369.9 nM FpGH4 (in 50 mM Tris/HCl, pH 7.4, 10004 NAD⁺, 1 mM MnCl₂) with varying concentrations of substrate (101 μM-5 mM) in a volume of 100 μl. The reactions were run as a series of three with two controls (no enzyme). The absorption (at 405 nm) resulting from pNP release by hydrolysis was monitored by Synergy H1™ plate reader [BioTek™] and converted to concentration using p-nitrophenol standard concentration curves determined under identical reaction conditions. Initial rates (μM/s) were determined and plotted in Grafit 7.0™ to determine the kinetic parameters.

FpGalNacDeacetylase

Michaelis-Menten parameters were determined for A antigen subtype 1ₚₑₙₜₐ-MU in 100 mM NaH₂PO₄, pH 7.4 at 37° C. using the coupled assays described previously (Kwan 2015). The assay was modified to allow detection of cleavage of the subtype 1 (and later 4), by use of BgaC (Jeong 2009) instead of BgaA (Singh 2014) as β-galactosidase. In addition, since A antigen subtype 1ₚₑₙₜₐ-MU contains an additional galactose, the concentration of BgaC was increased to 0.2 mg/mL to compensate for its need to cleave both the Gal-β-1,3-β-GlcNAc-β-1,3-Gal-β-MU and Gal-β-MU. Further, FpGalactosaminidase was included to allow the cleavage of the galactosamine-containing intermediate. Reaction setup in 100 μl was 3 nM FpGalNacDeacetylase (4.52 nM FpGalNacDeAc_D1ext, 3.55 nM FpGalNacDeAc_D1+2) and 0.01 mg/mL FpGalactosaminidase, 0.1 mg/mL SpHex, AfcA, 0.2 mg/mL BgaC and varying concentrations of substrate (5 μM-2.5 mM). The reactions were run as a series of four with controls (no FpGalNacDeacetylase) as duplicates. The fluorescence signal (365/435 nm) resulting from MU release by hydrolysis was monitored on a Synergy H1™ plate reader (BioTek™) and converted to concentration using MU standard concentration curves determined under identical reaction conditions. Initial rates (μM/s) were determined and plotted in Grafit 7.0 to determine the kinetic parameters.

k_{cat}/K_{M} parameter were determined for A antigen subtype 1/2/4ₜₑₜᵣₐ-MU at pH 7.4 at 37° C. Reactions (total volume of 100 μL) were performed in black 96-plate wells and as coupled assays in 100 mM NaH₂PO₄ (pH 7.4) with 12 nM FpGalNAcDeacetylase 0.1 mg/mL SpHex, BgaC (BgaA for subtype II), AfcA, at varying concentrations of substrate (25 μM, 20 μM, 15 μM, 10 μM, 7.5 μM, 5 μM). The reactions were run as a series of four with controls (no FpGalNAcDeacetylase) as duplicates. The fluorescence signal (365/435 nm) resulting from MU release by hydrolysis was monitored on a Synergy H1™ plate reader (BioTek™) and converted to concentration using MU standard concentration curves determined under identical reaction conditions. Initial rates (μM/s) were determined and plotted in Grafit™ 7.0 to determine the kcat/KM (s-1*mM-1) parameters.

GH109 Subtype Kinetic

kcat/KM parameter was determined for A antigen subtype 1/2/4ₜₑₜᵣₐ-MU at pH 7.4 and 37° C. Reactions (total volume of 100 μL) were performed in black 96-plate wells and performed as coupled assays in 100 mM NaH2PO4, pH 7.4 with 86.02 nM BvGH109_1/100.49 nM EmGfl109/80.52 nM BvGH109_2/87.4 nM BsGH109 and 5 μM NAD+, 0.1 mg/mL each of SpHex, BgaC (BgaA for Subtype 2), AfcA, varying concentrations of substrate (25 μM, 20 μM, 15 μM, 10 μM, 7.5 μM, 5 μM). The reactions were run as a series of four with controls (no α-N-acetylgalactosaminidase) as duplicates. The fluorescence signal (365/435 nm) resulting from MU release by hydrolysis was monitored by Synergy H1™ plate reader [BioTek™] and converted to concentration using MU standard concentration curves determined under identical reaction conditions. Initial rates (μM/s) were determined and plotted in Grafit 70.0™ to determine the kcat/KM (s-1*mM-1) parameters.

Crystallography

Prior to crystallization, FpGalNAcDeAc_D1ext was digested with thrombin (Novagen™) at a concentration of 1 mg/mL overnight using the manufacturer's suggested protocol. Protein was then purified by HisTrap FF column and the flow-through was collected, buffer-exchanged into 10 mM Tris pH 8.0+75 mM NaCl, and concentrated to 12 mg/mL.

Crystallization

FpGalNAcDeAc_D1ext (12 mg/mL) was crystallized by use of the hanging drop diffusion method using a reservoir solution composed of 0.2 M CaCl₂, 0.1 M MES pH 6, 18% PEG 4000, and 20 mM MnCl₂ at a 1:1 protein:reservoir ratio. A quick bromide soak was used to derivatize crystals for phasing and was prepared by transferring the crystal to a solution of 1M NaBr, 25% glycerol, 18% PEG4000, 20 mM CaCl⁻²), and 0.1 M Mes pH for 30 seconds and flash frozen in liquid nitrogen. Crystal complexes with blood group B antigen trisaccharide (B_tri) were prepared by pre-incubating protein (12 mg/mL) with m mM B_tri for 2 hours before setting up drops under the same conditions as above, but omitting MnCl₂. Crystals were cryoprotected with reservoir solution supplemented with 25% glycerol.

Data Collection, Phasing and Structure Determination

Datasets were collected at the Canadian Light Source™. Data were integrated using XDS (Kabsch 2010) and scaled with Aimless™ (Evans 2013). Phasing and automated structure solution was performed using CRANK2™ (Skubak 2013) in the CCP4I2™ program suite (Potterton 2018). The structure was checked and refined using alternating cycles of Coot™ (Emsley 2004) and Refmac™ (Vagin 2004). The B_tri structure complex was solved by difference Fourier and the ligand was manually built in Coot™ as were the water and metal ions. Difference density maps confirmed the presence of Mn²⁺ in the apo structure and Ca²⁺ in the liganded structure. Models were validated by Coot™ and Molprobity™ (Chen 2010). Atomic coordinates and structure factors of the apo and B_tri complex have been deposited in the Protein Data Bank (PDB) with accession numbers:

Flavonifractor plautii GalNAcDeacetylase Protein SEQ ID NO.: WP_009260926.1; and

Flavonifractor plautii Galactosaminidase Protein SEQ ID NO.: WP_044942952.1

Active-Site Mutagenesis

Based on structural information (not shown) and sequence alignment (not shown) FpGalNAcDeAc_D1min and FpGalNase_truncA were mutated using the QuickChange™ protocol (Zhang 2004), utilizing the primers noted in TABLE B. The mutants were purified via NiNTA and HIC columns as described above. The structural integrity of all mutants was checked via CD spectroscopy; all tested enzymes were structurally similar to their wild-type. For mutants with relatively low activity, reactions were carried out under the same conditions used for full kinetic determinations; however the substrate depletion method was used for determination of kcat/KM values as has been previously described (Vocadlo 2002). In brief: at low concentrations of substrate where [Substrate]<K_{M} (equivalent to ˜⅕- 1/10 of Kₘ) the k_{cat}/K_{M} value can be approximated upon non-linear fitting of the reaction time course to a first order curve and dividing by the enzyme concentration.

GH36 Phylogenetic Mapping

Reference sequences of GH36 were downloaded from the CAZy™ database using SACCHARIS™ cazy_extract.pl script (Jones 2018). Phylogenetic-based protein profiling software, TreeSAPP™ (available at https://github.com/hallamlab/TreeSAPP), was used to both build the reference trees and map the sequences to these trees. Briefly, HMMs from dbCAN were used to extract protein family domains from all full-length sequences downloaded from CAZy™ (Yin 2012). These sequences were then clustered at 70% sequence similarity using UCLUST™ to remove redundant sequence space and decrease the size of the tree (Edgar 2010). RAxML™ version 8.2.0 was used to build the reference trees with the ‘-autoMRE’ to decide when to quit bootstrapping before loon replicates have been performed, and PROTGAMMAAUTO™ to select the optimal protein model (Stamatakis 2006; and Stamatakis 2008).

TreeSAPP™ was then used to map the query sequences onto these reference trees. Briefly, protein sequences were aligned to HMMs using Hmmsearch™ and the aligned regions were extracted (Eddy 1998). Hmmalign™ was used to include the new query sequences in the reference multiple alignment and then TrimAl™ removed the unconserved positions from the alignment file (Capella-Gutierrez 2009). RAxML′ was used to classify the query sequences in the reference tree through insertions. Placements of each query sequence were filtered and concatenated into a single. Jplace™ file before being visualized in iTOL™ (Matsen 2012; and Letunic 2016).

RBC Assays

Whole blood from healthy consenting donors was collected into a citrate Vacutainer using a protocol approved by the clinical ethics committee of The University of British Columbia. The tube was spun at 1000×g for 4 min at RT, and RBCs were separated and washed 3 times with 1×PBS pH 7.4. For assays in the presence of dextran 40k, washed RBCs (200 μL, 10% Hematocrit) were placed in a tube, and the supernatant was partially removed and replaced with 1×PBS pH70.4 with and without dextran 40k (final concentration of 300 mg/mL). In addition some assays were performed in 1×PBS pH70.4+25% plasma or 100% plasma. RBCs were mixed carefully and placed on an orbital shaker for 30 s. Diluted enzyme solutions were then added, to a final volume of 200 μL. The tubes were vortexed very gently, and placed on an orbital shaker for defined times at set temperatures.

MTS Cards

After the reaction, RBCs were washed 3 times with an excess of 1×PBS pH 7.4 and analysed using Micro Typing System™ (MTS) cards [MTS′, Florida, USA]. RBCs (12 μl, 5% Hematocrit), suspended in diluent [MTS, Florida, USA], were added carefully to the mini gel column, leaving a space between the blood and the contents of the mini gel. The MTS cards were centrifuged at 156×g for 6 min at RT using a Beckman Coulter Allegra X-22R™ centrifuge with a modified sample holder as recommended. The extent of antigen removal from the surface of the RBC was evaluated from the location of RBCs in the mini gel after spinning, according to the manufacturer's instructions. RBCs with a high surface antigen concentration agglutinated upon interaction with the monoclonal antibody present in the gel column and could not penetrate (MTS™ score 4). RBCs with no surface antigens did not agglutinate and migrated to the bottom of the mini gel (MTS score 0). RBCs that underwent partial removal of surface antigens migrated to positions between these and were assigned scores between 0 (not present) and 4 (present) according to the manufacturer's instructions.

Agglutination Assays for H-Antigen

To analyse the conversion of A antigen to H antigen after enzymatic treatment, washed A-ECO-RBCs were mixed in equal parts with 2 μg/mL anti-H antibody (Anti-Blood Group H ab antigen antibody [97-I]: cat no. ab24213 (Abcam™)) and the appearance of agglutination within a 30 minutes time frame monitored. RBCs that underwent agglutination with the Anti-H antibody were assigned scores between 0 (no agglutination within 1800 sec) and 5 (agglutination within 120 sec).

FACS

Enzyme treated RBCs were washed 2× with 1×PBS pH 7.4 and 1% hematocrit ECO-RBCs were treated with 1/100 APC-anti-A antibody (Alexa Fluor™ 647 Mouse Anti-Human Blood Group A: cat no. 565384 (BD Pharmingen™)) and/or anti-H antibody (Anti-Blood Group H ab antigen antibody [97-I]: cat no. ab24213 (Abcam™)) for 30 minutes at RT, then washed 2× with 1×PBS pH7.4. For detection of the anti-H antibody a secondary FITC-labelled antibody (Goat F(ab′)2 Anti-Mouse IgM mu chain (FITC): cat no. ab5926 (Abcam™)) in a 1/500 concentration was used. The data were assessed after reconstitution into 1×PBS pH70.4 (1% hematocrit) with a flow cytometer (CytoFLEX™ (Beckman Coulter™)).

Enzyme Adsorption and Antigenicity

To test whether the enzymes can be readily removed from the RBCs after treatment, potential adsorption was assessed. Pacific blue-labelled FpGalNAcDeacetylase and FpGalNase (F/P=1) were incubated with the RBC's for 1 h at 37° C. alone, and after several wash steps, and then residual fluorescence measured on a flow cytometer (CytoFLEX™ (Beckman Coulter™)).

Antigenicity was tested by incubating RBCs with 50 μg/mL of each enzyme and mixing the enzyme treated RBCs with allogeneic or autologous serum, observing potential agglutination. Additionally, to assess potential Anti-IgG,-C3d exposure the treated RBCs were tested on Anti-IgG,-C3d MTS™ cards [MTS™, Florida, USA]. Incubation time was 30 minutes at 37° C.

Antigen Subtype's Synthesis

The synthesis of the A and B antigen subtypes 1/2/4tetra-MU was a performed with a modified protocol, described in Kwan (Kwan et al. 2015).

Two-Step H Antigen Subtype 1/2/4Tri-MU Synthesis

All three synthesis were performed in scales of 20 mg GalNAc-α-MU/GlcNAc-α-MU in 10 mL 50 mM Tris/HCl, 200 mM NaCl, pH 7.4, 10 mM MnCl₂, 50 U Alkaline Phosphorylase, 1.5 equivalent UDP-Gal, 1.2 equivalent GDP-Fuc (scaled on LacNAc-MU product). Depending on the desired product different glycosyl transferases in a concentration of 100 μg/mL were added; for subtype I CgtB S42 and Te2FT, for subtype II HP0826 and WbgL, for Subtype IV LgtD and Te2FT. The reaction was performed at 37° C. and the progress controlled via TLC (mobile phase, EtAc:MeOH:H₂O with a ratio of 6:2:1), the 4-Methylumbelliferone was hydrolysed from the compounds via 10% H₂SO₄ and detected via UV (360 nm). After no further product increase could be observed the reaction was applied to a HF Bond Elut C18 column, washed with several column volumes of 5% Methanol, and product was eluted with 25% Methanol. The solvent was then removed in vacuo.

A Antigen Subtype 1/2/4ₜₑₜᵣₐ-MU Synthesis

The final synthesis step was performed in scale of 10 mg H antigen subtype 1/2/4ₜᵣᵢ_MU in 5 mL 50 mM Tris/HCl, 200 mM NaCl, pH70.4, 10 mM MnC₂, 25 U Alkaline Phosphorylase, 1.5 equivalent UDP-GalNAc and 100 μg/mL BgtA at 37° C. The progress was followed via TLC, after no further product increase could be observed the reaction was applied to a HF Bond Elut C18 column, washed with several column volumes of 5% Methanol, and product was eluted with 25% Methanol. The solvent was then removed in vacuo. The final product was further purified on a 1.5×46 cm HW-40F size exclusion column and then freeze-dried.

B Antigen Subtype 1/2/4ₜₑₜᵣₐ-MU Synthesis

The final synthesis step was performed in scale of 10 mg H antigen subtype 1/2/4ₜᵣᵢ-MU in 5 mL 50 mM Tris/HCl, 200 mM NaCl, pH 7.4, 25 U Alkaline Phosphorylase, 1.5 equivalent UDP-Gal and 100 μg/mL BoGT6a at 37° C. The progress was followed via TLC, after no further product increase could be observed the reaction was applied to a HF Bond Elut C18 column, washed with several column volumes of 5% Methanol, and product was eluted with 25% Methanol. The solvent was then removed in vacuo. The final product was further purified on a 1.5×46 cm HW-40F size exclusion column and then freeze-dried.

GalN Antigen Subtype 1ₚₑₙₜₐ-MU Synthesis

10 mg of A antigen subtype 1-penta-MU were incubated with 1 μg/mL FpGalNAcDeacetylase in 5 mL 100 mM NaH₂PO₄ at 37° C. for 30 min and then stopped through addition of 1 mM EDTA. The complete conversion of the substrate was checked via TLC and the reaction applied to a HF Bond Elut C18 column, washed with several column volumes of 2% Methanol, and product was eluted with 10% Methanol. The solvent was then removed in vacuo.

Protein Purification

All proteins and there truncations were cloned via Golden Gate™ cloning (Engler 2008) or PIPE cloning (Mock 2008) into pET16b or pET28a. The primer sequences are set out in TABLE B.

The production of proteins for extended characterisation was performed in BL21(DE3) cells, cultured in 200 mL ZY5052 auto induction media (Studier 2005) for 20 h at 37° C., 220 rpm inoculated with 100 μl of an over-night LB culture. Cells were harvested by centrifugation (400 0×g, 40° C., 10 min) and resuspended in 10 mL lysis buffer (50 mM Tris/HCl, 150 mM NaCl, 1% (v/v) Glycerol, 40 mM Imidazol, pH7.4, 2 mM DTT, 1× Protease Inhibitor EDTA-free (Pierce™), 2 U Benzonase (Novagen™), 0.3 mg/mL Lysozyme, 10 mM MgCl₂), followed by sonification (3 min pulse time; 5 sec pulse, 10 sec pause, 35% amplitude) on ice. After removal of cell debris by centrifugation (14000×g. 4° C., 30 min), supernatant was collected and loaded on a nickel affinity chromatography column (5 mL HisTrap HP™ column (GE™)) using a peristaltic pump. The elution was performed and monitored on an AEKTApurifier™ system (GE™) with a 10-75% gradient of 50 mM Tris/HCl, 400 mM Imidazol, pH 7.4, 2 mM DTT, via SDS-PAGE the fractions containing the protein were identified and then pooled. Buffer exchange into 50 mM Tris/HCl, 150 mM NaCl, pH 7.4, 2 mM DTT and concentration was performed in Amicon Ultra-15 Centrifugal Filter Units™ MWCO 10 kDa (Millipore™).

FpGalNAcDeacetylase, FpGalactosaminidase and there truncations had to undergo a second round of purification, a Amicon Ultra-15 Centrifugal Filter Units™ MWCO 10 kDa (Millipore™) was used to exchange the buffers before loading the proteins on a hydrophobic interaction chromatography column (10 mL Phenyl Sepharose High Performance column (Pharmacia Biotech™)). Loading, washing and elution (gradient 0-100%) of the column was handled through an AEKTApurifier™ system (GE™), utilizing following buffer conditions: FpGalNAcDeacetylase; binding 1×PBS, 800 mM NH₂PO₄, pH 7.4 and elution 1×PBS, pH 7.4 and FpGalactosaminidase; binding 25 mM Tris/HCl, 1 M NaCl, pH70.4 and elution 25 mM Tris/HCl pH70.4. Via SDS-PAGE the fractions containing the protein were identified and then pooled. Buffer exchange into 50 mM Tris/HCl, 150 mM NaCl, pH7.4 and concentration was performed in Amicon Ultra-15 Centrifugal Filter Units™ MWCO 10 kDa (Millipore™).

Protein Characterization

Optimum pH Value

The general pH range for activity of FpGalNAcDeacetylase and FpGalactosaminidase for A antigen subtype 1ₚₑₙₜₐ-MU and GalN antigen subtype 1ₚₑₙₜₐ-MU, respectively was determined by product occurrence on TLC plates for varying pH values. The reaction was performed in 100 μl scales at 37° C. with 50 μM substrate and 1 μg/mL enzyme in the appropriate buffer system. Buffers for pH4 to 6 were based on a 50 mM citric acid/sodium citrate buffer, for pH 6-8 a 50 mM sodium phosphate buffer and pH 8-10 a 50 mM glycine/sodium hydroxide buffer.

To determine the optimal pH value 5 μg/mL FpGalactosaminidase was incubated in 100 μl 50 mM sodium phosphate buffer with varying pH range (50.8-8.0) and 200 μM GalN-α-pNP. The absorption (at 405 nm) resulting from pNP release was monitored by a Synergy H1™ plate reader (BioTek™) for 1 h at 37° C.

5 μg/mL FpGalNAcDeacetylase and 50 μM A antigen subtype Ipenta-MU was pre-incubated for 10 min at 37° C. in 25 mM sodium phosphate buffer with varying pH range (50.8-10.0). The reaction was quenched with 100 mM sodium phosphate buffer pH7.5, 100 μM EDTA, 5 μg/mL FpGalactosaminidase, 50 μg/mL SpHex, 50 μg/mL AfcA and 50 μg/mL BgaC, final volume 100 μl. The fluorescence signal (365/435 nm) resulting from MU release by hydrolysis was monitored by a Synergy H1™ plate reader (BioTek™) for 30 min at 37° C.

Protein Stability

FpGalNAcDeacetylase and FpGalNase were stored in 1×PBS buffer pH70.4 at 4° C. After 2 and 12 weeks, the activity of the enzymes were tested like described for the pH optimum against the A antigen subtype 1ₚₑₙₜₐ-MU in a coupled enzyme reaction for FpGalNAcDeacetylase and with GalN-α-pNP for FpGalNase.

FpGalNAcDeacetylase Inhibition

FpGalNAcDeacetylase was tested against different potential inhibitors in 96-well plate format as a coupled assay. Reaction was performed in 100 μL scale at 37° C. with 50 μM A antigen subtype 1penta-MU and 5 μg/mL FpGalNAcDeacetylase in 100 mM NaH₂PO₄ pH7.4 with 10 μg/mL FpGalactosaminidase, 50 μg/mL SpHex, 50 μg/mL AfcA, 50 μg/mL BgaC. As inhibitors EDTA (1, 10, 100 μM), Marimastat (1, 10, 100, 1000 μM), DMSO (2%, 4%), Protease Inhibitor Cocktail EDTA-free (Pierce™) (1×, 2× and 4×) were tested. The Fluorescence (365/435 nm) was monitored continuously for 1 hours using a Synergy H1™ plate reader (BioTek™). Additives showing strong effects were run again without the coupled enzymes and the product formation analysed via TLC.

Limited Proteolysis

To investigate if there are smaller, stable subdomains of FpGalactosaminidase, a limited proteolysis was performed. FpGalactosaminidase was treated with Thermolysin (10:1 protein:protease mass ratio) at various temperatures (20° C., 37° C., 42° C., 50° C., and 65° C.) for 1.5 hr. Samples were then run on an SDS-PAGE gel and a stable fragment was identified running around 70 kDa (down from the initial 118 kDa) with nearly complete digestion achieved at the 50° C. incubation temperature. This fragment was sent to the UBC proteomics core facility for peptide identification and was determined to be a C-terminal truncated version of the full length protein with cleavage site between amino acids 690-700.

Glycan Array Screening

For the glycan array screening 500 μg of FpGalNAcDeAc_D2ext were labeled with Fluorescein isothiocyanate (FITC) with a F/P ratio of 1 using the Fluorotag™ FITC conjugation Kit (Sigma™). The screening was performed in the CFG's Protein-Glycan Interaction Core Facility™ with version 5.3 of the printed array, consists of 600 glycans in replicates of 6 for 5 and 50 μg/mL protein concentration. Analysis of binding motifs was performed with the webtool at Emory University (https://glycopattern.emory.edu/).

Enzyme Testing in Buffered Extracellular Solutions

A composition comprising purified GalNAcDeacetylase enzyme (SEQ ID NO:5) and purified Galactosaminidase enzyme (SEQ ID NO:10) was used to test compatibility with buffered extracellular solutions PBS, Steen™ and Perfadex™ at 37° C., 37° C. and 4° C., respectively. Human type-A red blood cells (RBC) were incubated in various doses of enzyme composition in PBS, Steen™ and Perfadex™ to determine the ability of the enzymes to cleave A antigen from the red blood cells. A 1% RBC solution was treated with enzymes of various dosages in PBS, Steen™ and Perfadex™ solutions and the level of antigen removal at the end of treatment is analyzed by flow cytometry.

Immunohistochemical Analysis of Artery Biopsies

In order to test the dose escalation effects of the enzyme composition comprising purified GalNAcDeacetylase enzyme (SEQ ID NO:5) and purified Galactosaminidase enzyme (SEQ ID NO:10) was used to test type A human arteries in STEEN™ solution, the percent of type A antigen was quantified by immunohistochemical analysis of biopsies taken from untreated (control), treated (treatment) type A arteries and type O arteries as a negative control. Using area quantification software and normalized against the control group using the formula:

$$\% Normalized A ntigen = \frac{A r e {a}_{b t A} ( Treatment ) * \frac{A r e {a}_{total} ( Treatment )}{A r e {a}_{total} ( control )}}{A r e {a}_{b t A} ( Control )} .$$

Residual positive levels of type A antigen quantified in the type O group accounts for the artifacts occurred during process.

Enzymatic treatment on human artery was tested in human pulmonary artery (static treatment). The dose involved was prepared as a unit of weight of enzyme over volume of STEEN™ solution. The arteries were biopsied, processed and analyzed by immunohistochemistry with a double staining of CD31 (stains positive for endothelial cells) and BTA (stains positive for blood type A antigen). A 4-hour enzymatic treatment was done at both 11 μg/mL and 10 μg/mL on human artery. Artery biopsies immunohistochemical staining images at 20× magnification of artery treated without enzymes (Control) and artery treated with enzymes (Treatment). CD31 showed where endothelial cells (blood vessels) were located and BTA showed where blood type A antigen was located. The BTA in untreated arteries colocalized with endothelial cells (CD31 positives) and BTA was absent in the treated arteries.

Human Donor Lung Studies

Effects of 1-hour enzymatic treatment on ex vivo perfused human donor lungs, the expression level of type A antigen was quantified using immunohistochemical analysis of lung tissue biopsies and area quantification software, normalized against the Pre treatment biopsies using the formula:

$$\% Normalized Antigen = \frac{A r e {a}_{b t A} ( Post ) * \frac{A r e {a}_{total} ( Post )}{A r e {a}_{total} ( P re )}}{A r e {a}_{b t A} ( Pre )} .$$

The effects of i-hour and 3-hour enzymatic treatments (i.e. enzyme composition comprising purified GalNAcDeacetylase enzyme (SEQ ID NO:5) and purified Galactosaminidase enzyme (SEQ ID NO: 10)) on ex vivo perfused human donor lungs was tested. Immunohistochemical staining of biopsied human donor lungs was imaged at lox magnification to determine the effects of lung treated with the enzyme composition. CD31 shows where endothelial cells (blood vessels) are located; BTA shows where blood type A antigen are located. The pre-treatment images showed the blood type antigens locate both inside the blood vessels and air ways. In the post-treatment images the right upper dependent (RUD), right upper non-dependent (RUND), right middle non-dependent (RMND), right middle dependent (RMD), right lower non-dependent (RLND), and right lower dependent (RLD) areas of the lung, the blood type A antigens are absent in the blood vessels.

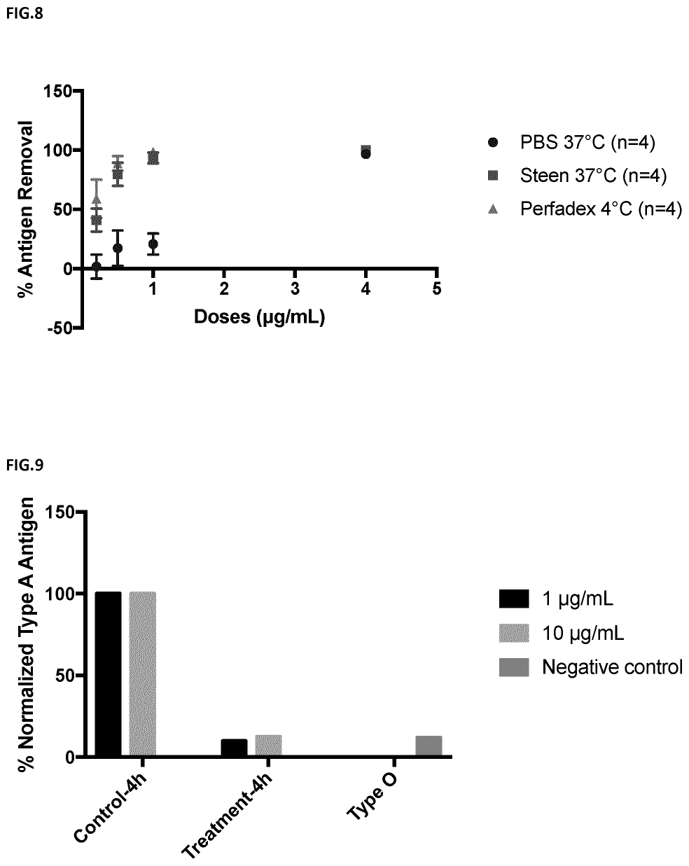

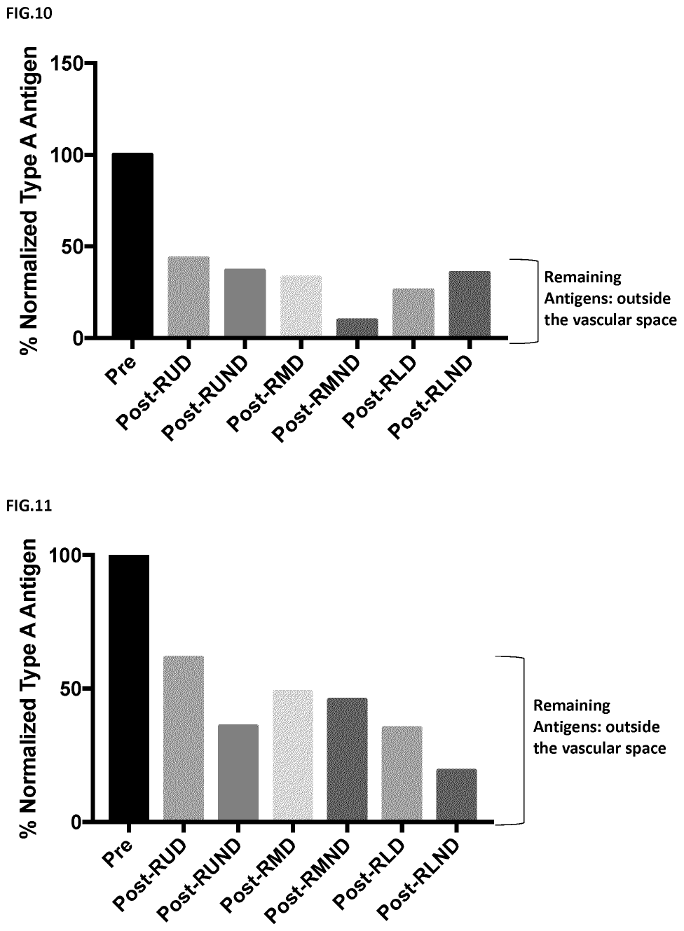

Two separate ex vivo perfused human donor lungs were tested in this study and the results are shown in FIGS. 10 and 11 at 1 and 3 hrs, respectively.

### EXAMPLES

### Example 1: Metagenomic Library Construction and Screening

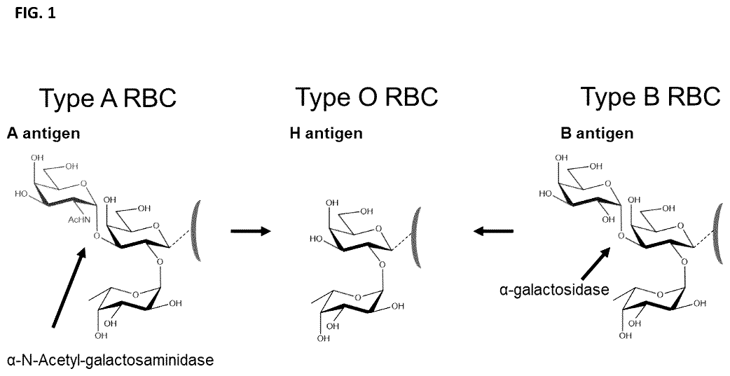

We constructed a metagenomic library that contains large (35-65 kb) fragments of DNA extracted from fecal samples provided by a male donor of AB⁺ blood type. Such a library contains multiple genes per bacterium, increasing the probability of expression of at least some of those genes and allowing expression of small “pathways” of multiple genes. Our library comprised ˜19,500 clones in 51×384 well plates, potentially around 800,000 genes, thus initial screening of such a library with expensive A-antigen substrates was impractical. Rather we first screened with simple, sensitive fluorogenic substrates—the methylumbelliferyl α-glycosides of galactose and N-acetyl-galactosamine (Gal-α-MU and GalNAc-α-MU). This initial screen, with a mixture of the two substrates, yielded a subset of 226 hits. These were re-screened against each individual substrate, identifying 44 with GalNAcase and 166 with galactosidase activity. A second round of screening was performed on these hits using the A-antigen and B-antigen tetrasaccharide glycoside substrates shown in FIG. 1, using a coupled enzyme assay (Kwan 2015), along with a no-substrate control: only if the initial Gal or GalNAc is cleaved can the coupling enzymes act and release MU. Eleven of these hits contained A-antigen cleaving activity, one of which also cleaved B-antigen, while six produced fluorescence in the absence of substrate thus encode pathways that generate unrelated fluorescent products.

### Example 2: Sequencing and Initial Analysis of Hits

The eleven fosmids were sequenced on an Illumina MiSeg™ and ORFs therein that are present in the CAZy™ database (http://www.cazy.org/)(Lombard 2014) were identified using Metapathways™ software (Konwar 2015). Due to the considerable depth of human microbiome sequencing now available, the organisms from which all fosmids were derived could be identified. Their sequences can be grouped into five clusters since eight of the eleven derived from overlapping fragments of the genomes of just two Bacteroides sp. The only gene common to all fosmids in cluster B is a GH109 enzyme (B. vulgatus); Cluster A also contains a GH109 (B. stercoris), while a GH109 is the only CAZy gene found in the other Bacteroides-derived fosmid (B. vulgatus). Fosmid No8, from the obligate anaerobe Flavonifractor plautii (Li 2015), contains three ORFs found within CAZy: an apparent carbohydrate binding module CBM32, and two potential glycoside hydrolases—a GH36 and a GH4. Finally fosmid K05 from a Collinsella sp., probably Collinsella tanakaei, contains no CAZy related ORFs. Here the generation of a sub-library of fosmid K05 allowed the identification of the ORF with A cleaving activity, later identified as a GH36 (not shown).

### Example 3: Analysis of the GH109 Enzymes

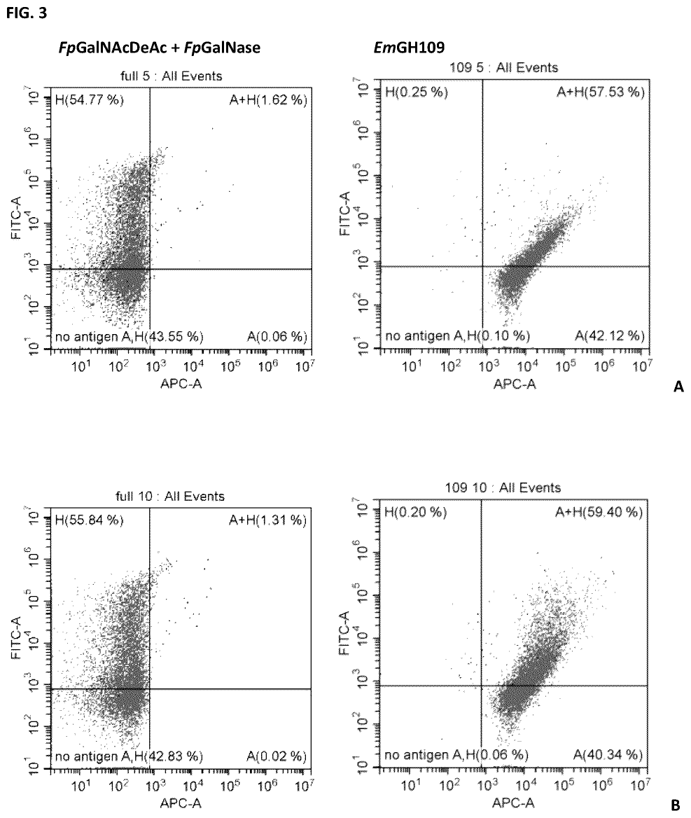

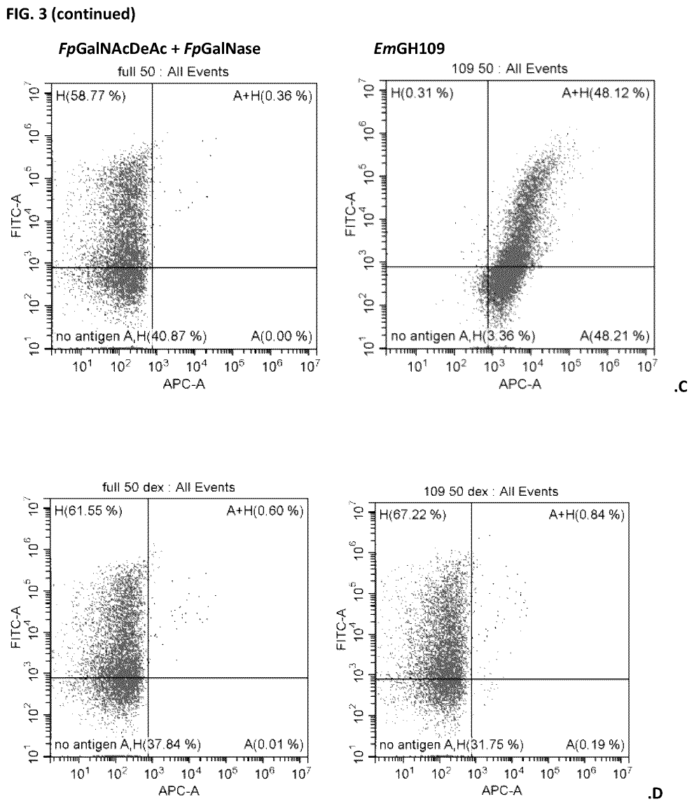

The GH109 family was founded on the basis of the A-antigen-cleaving activity of several of its members. These enzymes employ an unusual NAD⁺¬-dependent mechanism first uncovered in enzymes from GH4 Add Yip Ref (2004) J. Amer. Chem. Soc., 126, 8354-8355 as this was the one that showed the mechanism (Varrot 2005; and Liu 2007). The three GH109 genes identified here were cloned with a His tag after removal of signal peptides and expressed in Escherichia coli BL21(DE3). These three proteins, BsGH109, BvGH109_1 and BvGH109_2 (not shown), along with the canonical GH109 from Elizabethkingia meningosepticum (EmGH109) (Liu 2007) as a standard were purified and kinetic parameters for each determined. The three new enzymes displayed similar catalytic efficiencies with each of the three A-subtype substrates tested, largely mirroring the kinetic parameters of the EmGH109 standard. By contrast, when their A-antigen removal activity was tested on A⁺ RBCs using approved MTS cards, disappointingly only EmGH109 was significantly active. Testing was performed in the presence of Dextran 40K as a crowding agent, which we have shown to increase activity by concentrating enzyme on the cell surface (Chapanian 2014). In its absence, even at 150 ug/mL EmGH109 was ineffective, while in the presence of 300 mg/mL Dextran 40K, 15 μg/mL of enzyme was sufficient (see FIGS. 3 and 4). Previous studies showed that low ionic strength also boosted the activity of EmGH109 on cells (Liu 2007). Accordingly EmGH109 is not effective in whole blood.

### Example 4: Analysis of GH36 from Fosmid K05 from Collinsella sp

The identified GH36 protein within the Fosmid K05 (named K05GH36) was active towards GalNAc-α-MU and the A antigen tetrasaccharide. This is consistent with its membership of the GH36 family, which contains primarily α-galactosidases and α-N-acetyl galactosaminidases and carries out hydrolysis via a double displacement mechanism involving a covalent β-glycosyl enzyme intermediate (Comfort 2007). Phylogenetic analysis aligned its sequence within cluster 4 of the GH36 subfamilies (Fredslund 2011). Interestingly this cluster also contains, in close proximity, a characterized GH36 from Clostridium perfringens that is also known to cleave A antigen structures (Calcutt 2002). However, when we tested the ability of K05GH36 to remove A antigens from red blood cells its activity was disappointing, scoring only a 3, even when used in conjunction with a crowding agent.

### Example 5: Analysis of Fosmid No8 from Flavonifractor plautii

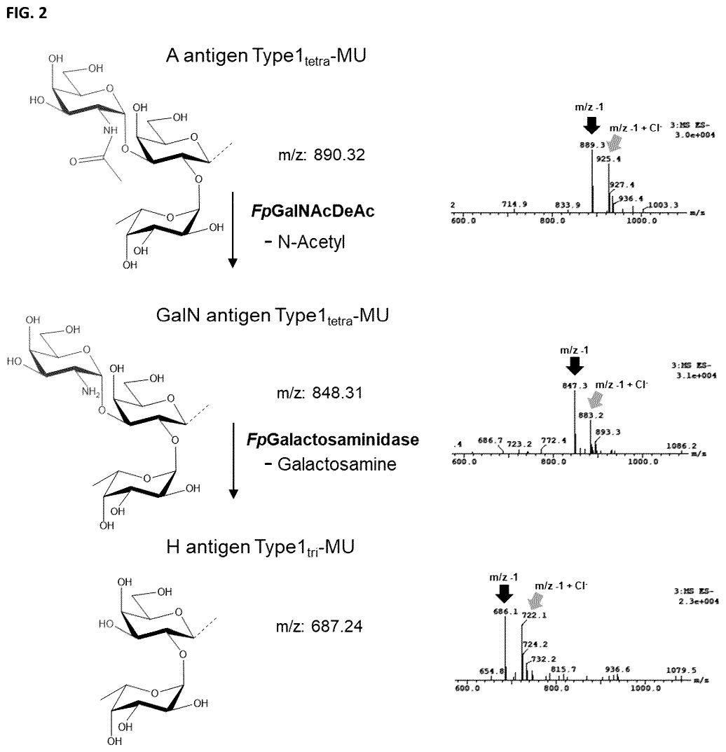

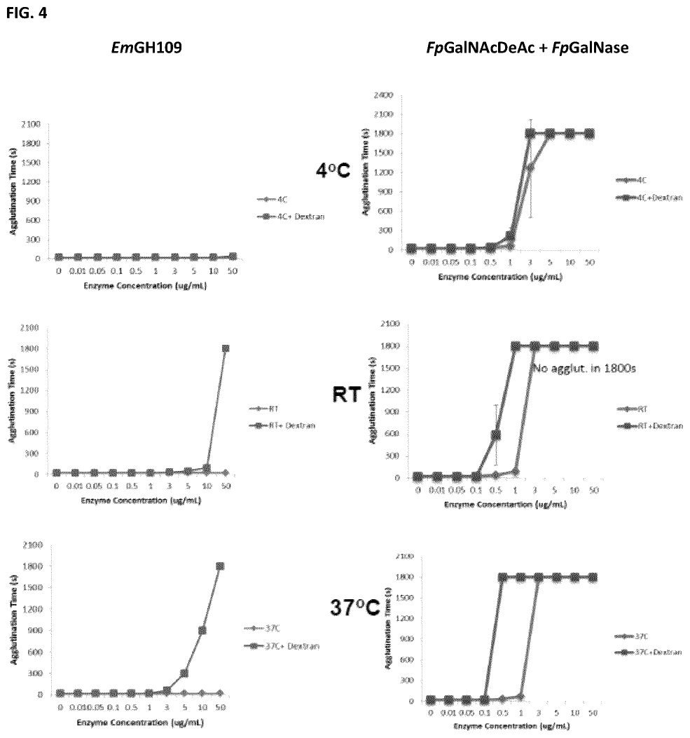

Since these new enzymes offered no advantages, our attention turned to the No8 fosmid from F. plautii, especially since its gene products cleave both A and B-antigens. The three CAZy-related genes were cloned, their signal peptide sequences removed, expressed in E. coli BL21(DE3) and the resulting enzymes purified in yields of up to 140 mg/L. Surprisingly, when we tested the individual purified proteins against the A and B tetrasaccharide substrates the only cleavage observed was of the B-antigen by N08GH36, with no cleavage of A-antigens by any of them. We therefore tested pairwise combinations of these enzymes and were surprised to discover that the mix of N08CBM32 and N08GH36 rapidly cleaved the A-antigen tetrasaccharide. TLC analysis of reaction mixtures with the individual enzymes revealed that N08CBM32 catalysed the conversion of A-antigen to a more polar but still UV-active product, while subsequent addition of N08GH36 released a sugar product that co-migrated with galactosamine, along with H antigen trisaccharide. MS analysis of reaction mixtures demonstrated that N08CBM32 is an A-antigen de-acetylase, hence the decrease of 42 in m/z and the more polar product, while N08GH36 is a galactosaminidase, a new activity for this family (FIG. 2). This was further confirmed by high performance anion exchange chromatography (HPAE-PAD) analysis of the reaction (FIG. 5), which showed that treatment of A-antigen with both enzymes released galactosamine, while the individual enzymes did not. Similar results were obtained with gastric mucin substrates, for which this enzyme presumably evolved. These two enzymes are therefore henceforth referred to as FpGalNAc deacetylase (FpGalNAcDeAc) and FpGalactosaminidase (FpGalNase).

While this pathway for degradation of the A-antigen was previously uncharacterised, fascinatingly it had been suggested over 50 years ago as an explanation for the so-called “acquired” B phenomenon wherein A-type patients infected with Clostridium tertium underwent an apparent change in blood type to type B (Gerbal 1975), as did forensic samples of human tissue that had been submerged in the river Thames (Ref Judd and Annesley https://doi.org/10.1016/S0887-7963(96)80087-3, Transfusion medicine reviews (1996) 10, 111-117). This presumably arose because the anti-B antibodies used in typing were unable to distinguish between terminal Gal and GalN.

Investigation of the third enzyme in the fosmid, the GH4, showed that while it hydrolyses Gal-α-pNP, GalN-α-pNP and GlcN-α-pNP, it does not cleave any A-antigen-based substrates. It therefore does not seem to play a direct role in conversion of A-antigen. However, these glycosaminidases do represent new activities within the GH4 family.

### Example 6: Characterisation of FpGalNAc Deacetylase

Closer bioinformatic analysis of this gene with Phyre2™ (Kelley 2015) indicated a 308 amino acid domain of previously unknown function at the N-terminus and an 145 amino acid CBM32 near the C-terminus, with linker regions between. Truncation analysis confirmed this basic structure since all constructs containing the intact deacetylase domain were indeed catalytically active (TABLE 2). This protein is therefore classified as the founding member of a new carbohydrate esterase family, CExx.

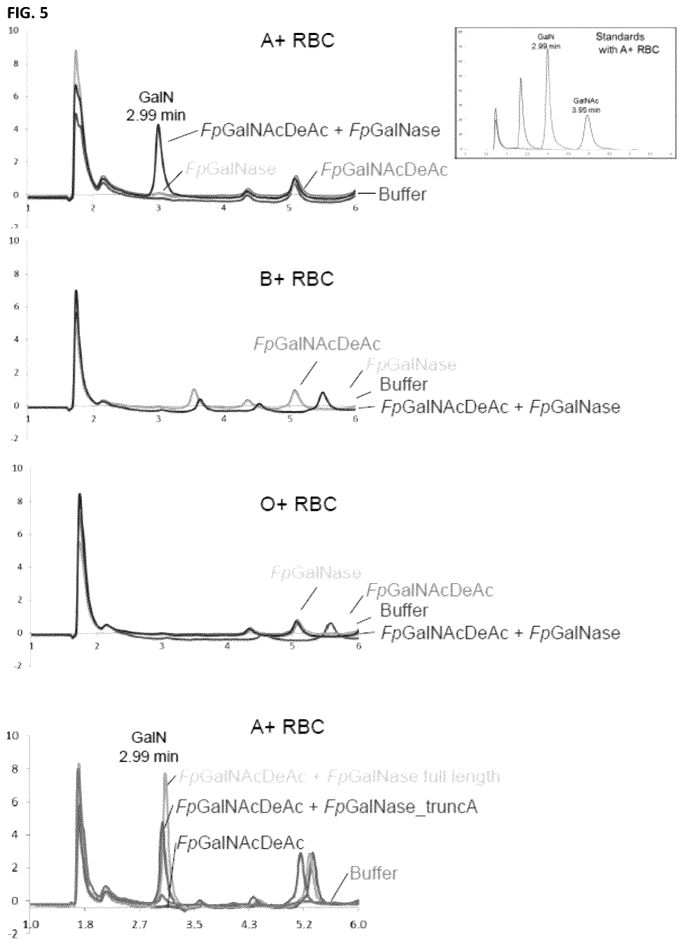

Acetamidosugar deacetylases have all proved to be metalloenzymes requiring divalent metal ions (Blair 2005). Consonant with this, treatment with 100 μM EDTA largely obliterated the enzyme activity, while addition of Mn²⁺, Co²⁺, Ni²⁺ or Zn²⁺ increased it. Other inhibitors of (non-metallo) amidases had no effect. The enzyme has a somewhat broad pH profile with an optimum around pH 8 (FIG. 6) and a narrow substrate specificity, restricted to the different A-subtypes and shorter versions thereof. However, within those sub-types it is not very discriminatory, there being only a 2-fold difference in specific activity between all of these sub-types (TABLE 2). Such a pH-dependence and specificity profile is ideal for RBC conversion since all subtypes of A are deacetylated, but nothing else.

The specificity of the CBM portion of the protein was explored using the glycan array of the Consortium for Functional Glycomics (CFG). The preferred targets were glycans with repeating N-acetyl lactosamine (LacNAc) structures, as also seen for the founding member of the CBM32 family; the N-acetylglucosaminidase from Clostridium perfringens (Ficko-Blean 2006). However, unlike that CBM, ours shows no high affinity binding to blood antigen structures. Repeating LacNAc structures are a common component of cell surfaces (Cohen 2009) as a universal component of complex and hybrid N-glycans, as well as some 0-glycans and glycolipids. In our case they presumably serve as the anchor point for attachment of the deacetylase domain. This would bring its catalytic domain into close proximity to the A-antigen without competition for its own substrate. In support of this model, removal of the domain resulted in a decreased activity on RBC's, with no effect on rates of soluble substrate cleavage (TABLE 2).

### Example 7: Crystallographic Analysis of FpGalNAc Deacetylase

To provide structural insight into this novel enzyme activity the truncated proteins were subjected to crystallisation trials and FpGalNAcDeAc_D1ext found to produce the crystals that diffracted to the best resolution. Solution of this structure revealed a catalytic domain that adopts a 5-fold beta propeller structure with an active site harbouring a divalent metal ion coordinated by D100 and H252. Co-crystallization of the enzyme with B-antigen trisaccharide as a close analogue of the reaction product unveiled its binding mode. At the base of the active site pocket, the non-reducing end galactosyl moiety, which is the distinguishing group between A-antigen and B-antigen, makes hydrogen bonding interactions with H97, E64 and two of the metal coordinated waters. The rest of the ligand is surface-exposed and polar interactions are identified between the fucosyl group and the S61 and D121 sidechains. The C1-OH group of the reducing end galactosyl moiety is solvent exposed, thus extensions to the substrate (i.e. with GlcNAc) are readily accommodated by the enzyme. Modelling of the N-acetyl group of the A-trisaccharide onto this structure allowed us to make rational mutations of the nearby amino acids, potentially involved in substrate deacetylation. The residue E64 proved to be critical for activity since both mutants were inactive, suggesting a direct role, probably in activation of the nucleophilic water molecule (TABLE 1). The residues that coordinate the divalent metal, D100, Y315 and H252 also proved to be important, with mutation of any resulting in ˜5000-fold rate decreases, consistent with their apparent role in binding the divalent metal ions. By analogy to other acetamidosugar deacetylases we propose that FpGalNAc deacetylase carries out hydrolysis by the mechanism, wherein the metal serves to polarize the carbonyl and activate a water molecule for nucleophilic attack on the carbonyl to form the tetrahedral intermediate. Decomposition of that intermediate is facilitated by proton donation to the sugar nitrogen atom by His 100.

TABLE 1
Specific activity of FpGalNAcDeAc_D1min its
mutants for the cleavage of A antigen Type2ₜₑₜᵣₐ-MU

| mutation site | k_{cat}/K_{M} [min⁻¹*mM⁻¹] |
|---|---|
| WT | 492.751 ± 124.002 |
| E64A | N.D. |
| E64L | N.D. |
| C99A | 140.680 ± 5.798 |
| C99S | 75.595 ± 2.491 |
| D100N | 0.073 ± 0.005 |
| H252A | 0.084 ± 0.012 |
| H252F | 0.030 ± 0.002 |
| Y315F | 0.167 ± 0.039 |

N.D. = no detectable activity

### Example 8: Characterisation of FpGalNAcDeAc and FpGalNase

Phylogenetic analysis of the sequence places FpGalNase in a new subgroup (5) of the GH36 family (Fredslund 2011). The 390 amino acid catalytic domain is located in the centre of this large (1079 amino acid) protein, with a potential carbohydrate binding domain at the C terminus. Removal of this C-terminal domain had no effect on kinetic parameters of the enzyme with soluble substrates (TABLE 2), but led to reduced efficiency in cleavage of deacetylated A⁺ RBCs. The enzyme is specific for galactosamine-containing sugars and will not cleave GalNAc residues in any context tested. However, it has a fairly broad specificity for cleavage of de-N-acetylated galactosaminides ranging from the simple aryl glycosides GalN-α-pNP upwards. Indeed (TABLE 2) k_{cat}/K_{M} values for the three A subtypes tested were all similar to each other and to those of the deacetylase. Values of k_{cat}/K_{M} for cleavage of B-antigen were over 2000 times lower than for the corresponding GalN-antigen, but nonetheless were sufficient to yield a positive hit on the original screen. This specificity for de-acetylated alpha galacto-configured substrates, coupled with its pH optimum of ˜6.5-7.0 suit it well for use in blood type conversion in conjunction with the deacetylase (FIG. 6).

TABLE 2
Kinetic parameters of FpGalNAcDeAc and FpGalNase
constructs for different antigen substrates

| | | | K_{M} | k_{cat} | k_{cat}/K_{M} |
|---|---|---|---|---|---|
| | | Substrate | [μM] | [s⁻¹] | [s⁻¹*mM⁻¹] |
| FpGalNAcDeAc | full length | A antigen Type1ₚₑₙₜₐ-MU | 340 ± 35 | 4.86 | 14.28 |
| | _D1ext | A antigen Type1ₚₑₙₜₐ-MU | 212 ± 37 | 5.37 | 25.23 |
| | _D1 + 2 | A antigen Type1ₚₑₙₜₐ-MU | 265 ± 43 | 9.99 | 37.59 |
| | full length | A antigen Type1ₜₑₜᵣₐ-MU | — | — | 23.49 ± 0.63 |
| | full length | A antigen Type2ₜₑₜᵣₐ-MU | — | — | 23.37 ± 0.71 |
| | full length | A antigen Type4ₜₑₜᵣₐ-MU | — | — | 33.27 ± 2.08 |
| FpGalNase | full length | GalN antigen Type1ₚₑₙₜₐ-MU | 64.5 ± 7.6 | 2.35 | 36.48 |
| | _truncA | GalN antigen Type1ₚₑₙₜₐ-MU | 54.7 ± 3.1 | 1.86 | 34.08 |
| | full length | GalN antigen Type1ₜₑₜᵣₐ-MU | — | — | 45.70 ± 3.57 |
| | full length | GalN antigenType2ₜₑₜᵣₐ-MU | — | — | 47.27 ± 7.49 |
| | full length | GalN antigen Type4ₜₑₜᵣₐ-MU | — | — | 30.05 ± 2.33 |
| | full length | B antigen Type1ₜₑₜᵣₐ-MU | — | — | 0.02 |
| | full length | GalN-α-pNP | 700 ± 151 | 0.029 | 0.04 |

### Example 9: Cleavage of A-Antigen from RBCs

Type A⁺, B⁺ and O⁺ RBCs were incubated with FpGalNAcDeAc and FpGalNase, individually and as a mixture and the released sugars analysed on a HPAE-PAD ion chromatogram. Neither of the enzymes used individually released any sugar products. However, when the mixture of the two was employed, galactosamine was clearly released from Type A⁺ RBCs but not from B⁺ or O⁺, proving a high specificity towards only the A antigen. This is very important as it shows that GalNAc is not released from the RBC surface in any other context. The truncated version of FpGalNase was also effective, but with slightly lower activity.

We then moved on to testing for antigen removal from RBCs using the industry standard MTS' cards. These antibody-conjugated columns are loaded with RBCs and spun in a centrifuge. Antigen-free RBCs migrate to the bottom of the column and are scored as 0, while untreated RBCs bearing the corresponding antigen stick at the top and are scored as 4, with intermediate scores ranking the degree of antigen removal. Treatment with FpGalNase alone did not remove A or B antigenicity at the concentration employed (TABLE 3) consistent with its inactivity on GalNAc substrates, and its low activity on Gal. Incubation with FpGalNAcDeAc removed antigenicity due to conversion of the acetamide to an amine, compromises binding of the Anti-A antibody employed. The minimal amount of enzyme required for complete antigen de-acetylation was assessed for FpGalNAcDeAc alone and in combination with FpGalNase, both in the absence and presence of 300 mg/ml Dextran as crowding agent. Amounts of FpGalNase down to 3 μg/ml were sufficient without assistance from Dextran, while inclusion of 300 mg/ml dextran reduced the required loading to 0.5 μg/ml (TABLE 3). By comparison the best previous enzyme, EmGH109 was ineffective in the absence of Dextran, unless low salt buffers were employed, while in the presence of dextran the minimum effective concentration was 15 μg/ml, a 30-fold higher loading. Versions of FpGalNAcDeAc missing the CBM were much less effective.

TABLE 3
MTS card results for treatment of A⁺, B⁺ and AB⁺
RBCs with EmGH109, FpGalNAcDeAc and FpGalNase.

| | | | Enzyme | | |
|---|---|---|---|---|---|
| | Blood | Dextran | cone, | Anti-A | Anti-B |
| Enzyme | type | 40k | [μg/ml] | MTS | MTS |
| none | A⁺ | | | 4 | |
| none | B⁺ | | | | 4 |
| none | AB⁺ | | | 4 | 4 |
| EmGH109 | A⁺ | 300 mg/ml | 15 | 0 | |
| FpGalNAcDeAc | A⁺ | | 3 | 0 | |
| FpGalNAcDeAc | A⁺ | | 2 | 1 | |
| FpGalNAcDeAc | A⁺ | 300 mg/ml | 0.5 | 0 | |
| FpGalNAcDeAc | A⁺ | 300 mg/ml | 0.4 | 2 | |
| FpGalNase | B⁺ | | 100 | | 4 |
| FpGalNase | B⁺ | 300 mg/ml | 100 | | 4 |
| FpGalNase | AB⁺ | | 100 | 4 | 4 |
| FpGalNase | AB⁺ | 300 mg/ml | 100 | 4 | 4 |

Since the MTS' card test on its does not assess the complete conversion of the A antigen and since no antibody was available to detect the GalN antigen we focused on the detection of newly formed H antigens on the treated RBC's. FpGalNase was functional at a concentration of only 5 μg/ml, leading to an increase of H-antigen level in concert with loss of A-antigen, as confirmed by FACS analysis seen in FIG. 3. By measuring agglutination times in the presence of Anti-H-antibodies we demonstrated the functionality of both enzymes for several A⁺ RBC donors, also in whole blood reaction conditions, an ability no other blood converting enzyme achieved before. Thus this pair of enzymes converts A⁺ RBCs to O-type “universal donor” RBCs using much lower enzyme loadings than required for the best previous enzymes. However, before transfusion of these RBCs into a patient, removal of all traces of the enzymes used in conversion to avoid adverse immune response, most likely by washing cells after centrifugation is advised. To confirm that this could be achieved we treated A⁺ RBCs with a fluorescently labelled sample of FpGalNAcDeAc and FpGalNase, then used FACS analysis to confirm that indeed simple washing was effective (FIG. 3).

Further characterization of the produced A-ECO RBC's may be useful to assess their full viability for usage in transfusion medicine, but the possibility to including the enzymes directly in to the blood plasma, potentially while collecting the blood donation, may allow an easy and cost efficient implementation off the process into the already existing automated routines of the blood collection and storage. In particular, the stability of the enzymes was tested as shown in TABLE 4.

TABLE 4
Storage Stability for Galactosaminidase and a GalNAcDeacetylase

| protein | time | Samples [mU] | | |
|---|---|---|---|---|
| FpGalNase | fresh sample 1 | 0.492 | 0.494 | 0.502 |
| | fresh sample 2 | 0.425 | 0.437 | 0.439 |
| | 2 weeks stored at 4° C. | 0.480 | 0.454 | 0.468 |
| | 12 weeks stored at 4° C. | 0.494 | 0.497 | 0.499 |
| FpGalNAcDeAc | fresh sample 1 | 888 | 840 | 888 |
| | 2 weeks stored at 4° C. | 864 | 840 | 936 |
| | 12 weeks stored at 4° C. | 864 | 840 | 888 |

Substrate: 100 μM GalN-α-pNP
Substrate: 100 uM A antigen T1ₜₑₜᵣₐ-MU

### Example 10: GalNAcdeacetylase and Galactosaminidase Fusion from Clostridium tertium

In looking for similar enzymes a novel Clostridium tertium natural fusion of a Galactosaminidase and GalNAcDeacetylase connected by a CBM (GH36_domain-CBM-Deacetylation_domain) was identified. Initial testing showed that the enzyme cleaves the A antigen (same mechanism, first deacetylation then galactosamine cleavage) of red blood cells, but not as efficiently (i.e. similar to the EmGH109). The Clostridium tertium deacetylation domain is not as efficient as the F. plautii GalNAcDeacetylase, but if subsidized with the F. plautii GalNAcDeacetylase the Clostridium tertium Galactosaminidase domain shows similar activity to F. plautii Galactosaminidase on red blood cells.

### Example 11: Alternative GalNAcdeacetylase and Galactosaminidase Enzymes

Data shows that the Clostridium tertium Galactosaminidase (Ct5757_GalNAse) and Rp1021 do have comparable enzyme activity for the conversion of GalN antigen to H antigen (2nd reaction step).

Data was also collected for alternative GalNAcdeacetylase and Galactosaminidase enzymes and the alternative enzymes were compared to the Flavonifractor plautii GalNAcDeacetylase and Flavonifractor plautii Galactosaminidase. As shown in TABLE 5, the MTS scores for anti-A antibodies on treated A RBC are shown for Clostridium tertium natural fusion of a Galactosaminidase and GalNAcDeacetylase, which requires the presence of Dextran to effectively cleave A antigen, and also shows good activity Clostridium tertium GalNAcDeacetylase (Ct5757_DeAcase) when combined with Flavonifractor plautii Galactosaminidase (FpGalNase). Also in TABLE 6, the data shows that Robinsoniella peoriensis (Rp) Rp3672 and Rp3671 are able to deacetylate the A antigen on RBCs, but are less efficient then FpGalNAcDeAcase and activity was only achieved in the presence of a crowding agent (i.e. Dextran 40 k).

TABLE 5
MTS scores for anti-A antibodies on treated A RBC

| Sample | Anti-A MTS Score |
|---|---|
| A RBC Control | 4 |
| FpGalNAcDeAcase + FpGalNase (10 μg/mL) | 0 |
| Ct5757 (10 μg/mL) | 4 |
| Ct5757 (50 μg/mL) + Dextran 40k | 0 |
| Ct5757_DeAcase + FpGalNase (10 μg/mL) | 0 |

TABLE 6
Robinsortiella peoriensis (Rp) 3671 and 3672 MTS scores

| Sample | Anti-A MTS Score |
|---|---|
| A RBC Control | 4 |
| RP3671 (50 μg/mL) + Dextran 40k | 3 |
| RP3672 (50 μg/mL) + Dextran 40k | 1 |

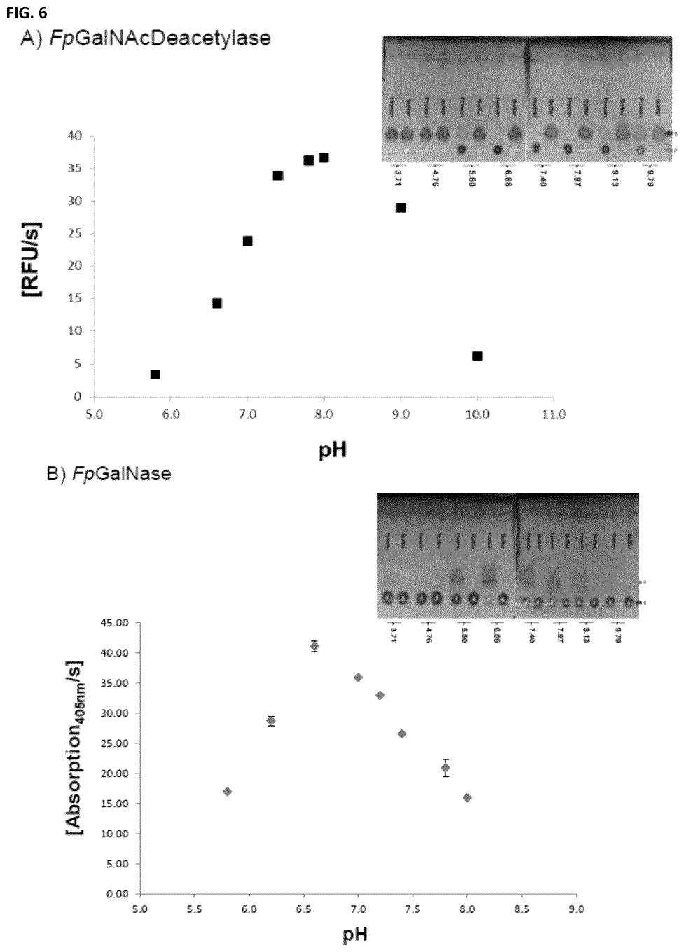

FIG. 7 shows conversion of A antigen to H antigen on A RBCs as analysed via FACS sorting, for (A) A+ RBC control, (B) Flavonifractor plautii GalNAcDeacetylase (FpGalNAcDeAc)+Flavonifractor plautii Galactosaminidase (FpGalNase) (10 μg/mL), (C) FpGalNAcDeAc+Clostridium tertium (Ct) Ct5757_GalNase (10 μg/mL) and (D) FpGalNAcDeAc+Robinsoniella peoriensis (Rp) Galactosaminidase (Rp1021) GalNase (10 μg/mL). The data shows that Clostridium tertium (Ct) Ct5757_GalNase and Robinsoniella peoriensis (Rp) Galactosaminidase (Rp1021) GalNase have comparable enzyme activity to Flavonifractor plautii Galactosaminidase (FpGalNase) for the conversion of GalN antigen to H antigen (2^{nd} reaction step).

### Example 12: Compatibility of the Enzyme Composition with Perfusion/Preservation Fluids

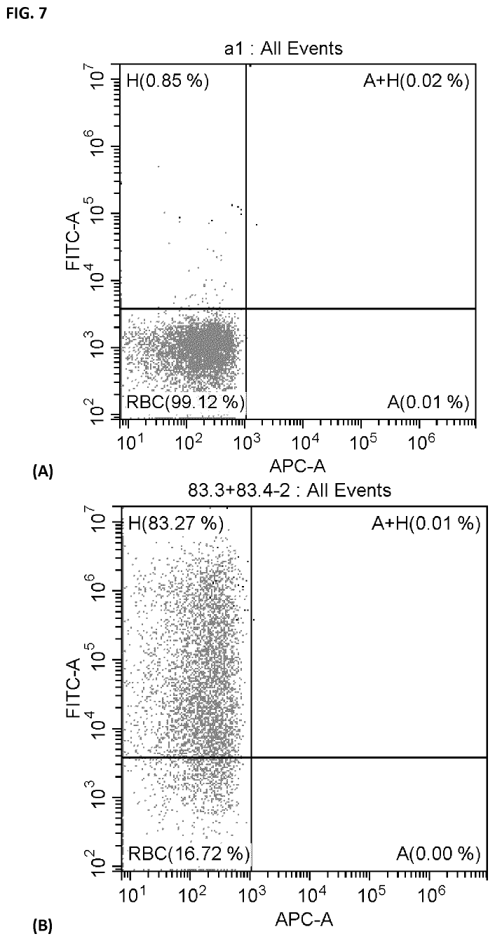

To ensure that the enzyme composition was compatible with the EVLP system, we first tested the function of enzymes (purified Flavonifractor plautii GalNAcDeacetylase protein of SEQ ID NO.:5 and purified Flavonifractor plautii Galactosaminidase protein of SEQ ID NO.:10) in organ perfusion/preservation fluids (STEEN™ and Perfadex™, XVIVO perfusion). The compatibility was evaluated based on the enzyme compositions' ability to remove blood type A antigens on red cell in STEEN™ at 37° C. or Perfadex™ at 4° C. Phosphate buffered saline (PBS) at 37° C. was used as a comparison group, as PBS is one of the standard solutions used for the blood treatment. The temperatures studied for STEEN™ and Perfadex™ were based on their working temperatures in clinical practice. The level of antigen removal was analyzed by flow cytometry. A dose escalation study in STEEN™ and Perfadex™ was carried out to help predict the appropriate dose to be used in organs (see FIG. 8). The unit of dose used throughout the study is defined as weight of enzymes (itg) over volume of solution (mL).

It was shown that the enzyme composition was fully compatible with STEEN™ and Perfadex™ perfusion/preservation fluids, the perfusion/preservation fluids enhanced the enzyme compositions' efficiency as compared to PBS. The enzyme composition was able to remove over 90% of antigen in STEEN™ and Perfadex™ at the total enzyme concentration of 1 μg/mL, while the same effect in PBS was achieved at the dose of 4 μg/mL (FIG. 8).

### Example 13: Static Treatment of Human Arteries

To test the efficacy of the enzymes (purified Flavonifractor plautii GalNAcDeacetylase protein of SEQ ID NO.:5 and purified Flavonifractor plautii Galactosaminidase protein of SEQ ID NO.:10) on the tissue level, an in vitro model of the human artery was used. Pulmonary arteries from the same human donor were split into a control group (STEEN™ solution) and treatment group (enzyme composition+STEEN™ solution), and incubated statically at 37° C. for 4 hours. Biopsies were taken at the end of incubation for both groups. The enzyme compositions were dosed at 1 μg/mL and 10 μg/mL. The change in blood type antigen was analyzed by immunohistochemistry. Consecutive sections of the biopsies were double-stained with CD31. (a marker of endothelial cells) to show the location of inner surface of blood vessel and BTA to show the expression of blood type antigen.

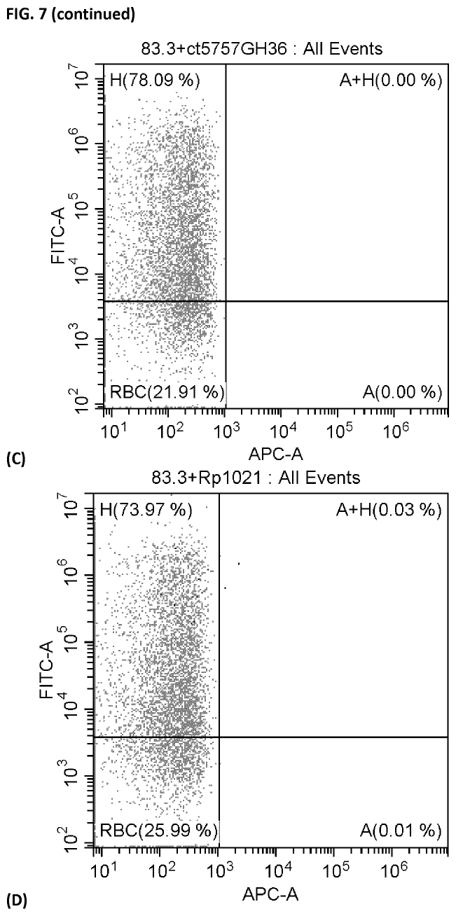

The expression level of blood type A antigen in the treatment group was significantly reduced as compared to the control group. The dose effect of 1 μg/mL and 1.0 μg/mL were similar the treated arteries. It is possible that at a total enzyme concentration (dose) lower than 1 μg/mL, the enzyme will also work. The disappearance of blood type antigen was confirmed when comparing the staining images of BTA to CD31. (FIG. 9).

### Example 14: Ex Vivo Perfusion of Human Lungs

The efficacy of enzyme-containing STEEN™ solution in removing histo-blood type antigens in human organs (for example, lungs) was tested under the Toronto EVLP setting. Donor human were lungs assessed with clinical ex vivo lung perfusion (EVLP) and were determined as unsuitable for transplantation and thus suitable for testing of the enzyme composition. After lungs were declined, the enzyme composition (purified Flavonifractor plautii GalNAcDeacetylase protein of SEQ ID NO.:5 and purified Flavonifractor plautii Galactosaminidase protein of SEQ ID NO.:10) was added to the STEEN™ perfusion fluid to initiate the treatment. A dose of 1 μg/mL was used. Biopsies were taken before and after treatment. The change in expression of blood type antigen was analyzed by immunohistochemistry. The function and physiology of lungs was also monitored hourly throughout the experiment to ensure no acute side effects were caused by the treatment.

For human lungs, the volume of perfusion liquid required is 1.5 L for single lung EVLP and 2 L for double lung EVLP. In the first test (FIG. 10) a single right lung EVLP, 1.5 mg of enzyme composition was added to perfusion fluid to reach the 1 μg/mL dose. The lung was treated for one (i) hour. Immunohistochemical analysis showed marked decreases in the level of blood type A antigen post treatment (FIG. 10). A comparison of sections of the pre-treatment biopsy, which was double-stained for blood type antigen and blood vessel, revealed that the antigens in lungs are located not only on the surface of blood vessel wall but also in airways. Comparison of the double-stained post-treatment biopsy indicates that the intravascular antigens were effectively removed.

In the second test (FIG. 11) a different single right lung EVLP was treated with 1.5 mg of enzyme composition in STEEN™ perfusion fluid to reach the concentration of 1 μg/mL. The lung was treated for three (3) hours. Immunohistochemical analysis showed that the expression level of blood type A antigen was markedly decreased. A comparison of the pre-treatment biopsy, that was double-stained for blood type antigen and blood vessel, revealed that the blood type antigens in lungs are located not only on the surface of blood vessels but also in airways (FIG. 11). Comparison of the double-stained post-treatment biopsy indicates that the intravascular antigens were effectively removed (FIG. 11). No acute side effect was observed in the lung's physiology and function after initiation of the enzymatic treatment.

The results show that at the dose of 1 μg/mL, the enzymes take effect in perfused human lungs within an hour.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. The invention includes all embodiments and variations substantially as hereinbefore described and with reference to the examples and drawings.

### Sequences

The Flavonifractor plautii DNA sequences were modified from the naturally occurring DNA seq (GalNAcDeacetylase 2311/2319nt/Galactosaminidase 3228/3237nt). In particular, there is a difference in the length of the sequences used for protein purification, whereby the signal peptides was removed and a N-terminal HisTag was added through the vector backbone.

INFORMAL SEQUENCE LISTING
Description: Flavonifractor plautii GalNAcDeacetylase (Protein seq)
SEQ ID NO: 2
MRNRRKAVSLLTGLLVTAQLFPTAALAADSSESALNKAPGYQDFPAYYSDSAHADDQVTHPDVVVLEEPWNGYRYWAVYTPNV
MRISIYENPSIVASSDGVHWVEPEGLSNPIEPQPPSTRYHNCDADMVYNAEYDAMMAYWNWADDQGGGVGAEVRLRISYDGVH
WGVPVTYDEMTRVWSKPTSDAERQVADGEDDFITAIASPDRYDMLSPTIVYDDFRDVFILWANNTGDVGYQNGQANFVEMRYS
DDGITWGEPVRVNGFLGLDENGQQLAPWHQDVQYVPDLKEFVCISQCFAGRNPDGSVLHLTTSKDGVNWEQVGTKPLLSPGPD
GSWDDFQIYRSSFYYEPGSSAGDGTMRVWYSALQKDTNNKMVADSSGNLTIQAKSEDDRIWRIGYAENSFVEMMRVLLDDPGY
TTPALVSGNSLMLSAETTSLPTGDVMKLETSFAPVDTSDQVVKYTSSDPDVATVDEFGTITGVSVGSARIMAETREGLSDDLE
IAVVENPYTLIPQSNMTATATSVYGGTTEGPASNVLDGNVRTIWHTNYAPKDELPQSITVSFDQPYTVGRFVYTPRQNGTNGI
ISEYELYAIHQDGSKDLVASGSDWALDAKDKTVSFAPVEAVGLELKAIAGAGGFGTAAELNVYAYGPIEPAPVYVPVDDRDAS
LVFTGAWNSDSNGSFYEGTARYTNEIGASVEFTFVGTAIRWYGQNDVNFGAAEVYVDGVLAGEVNVYGPAAAQQLLFEADGLA
YGKHTIRIVCVSPVVDFDYFSYVGE
Description: Flavonifractor plautii GalNAcDeacetylase (removed
signal peptide Protein seq)
SEQ ID NO: 4
ADSSESALNKAPGYQDFPAYYSDSAHADDQVTHPDVVVLEEPWNGYRYWAVYTPNVMRISIYENPSIVASSDGVHWVEPEGLS
NPIEPQPPSTRYHNCDADMVYNAEYDAMMAYWNWADDQGGGVGAEVRLRISYDGVHWGVPVTYDEMTRVWSKPTSDAERQVAD
GEDDFITAIASPDRYDMLSPTIVYDDFRDVFILWANNTGDVGYQNGQANFVEMRYSDDGITWGEPVRVNGFLGLDENGQQLAP
WHQDVQYVPDLKEFVCISQCFAGRNPDGSVLHLTTSKDGVNWEQVGTKPLLSPGPDGSWDDFQIYRSSFYYEPGSSAGDGTMR
VWYSALQKDTNNKMVADSSGNLTIQAKSEDDRIWRIGYAENSFVEMMRVLLDDPGYTTPALVSGNSLMLSAETTSLPTGDVMK
LETSFAPVDTSDQVVKYTSSDPDVATVDEFGTITGVSVGSARIMAETREGLSDDLEIAVVENPYTLIPQSNMTATATSVYGGT
TEGPASNVLDGNVRTIWHTNYAPKDELPQSITVSFDQPYTVGRFVYTPRQNGTNGIISEYELYAIHQDGSKDLVASGSDWALD
AKDKTVSFAPVEAVGLELKAIAGAGGFGTAAELNVYAYGPIEPAPVYVPVDDRDASLVFTGAWNSDSNGSFYEGTARYTNEIG
ASVEFTFVGTAIRWYGQNDVNFGAAEVYVDGVLAGEVNVYGPAAAQQLLFEADGLAYGKHTIRIVCVSPVVDFDYFSYVGE
Description: Flavonifractor plautii GalNAcDeacetylase with HisTag
(pET16a-Protein seq)
SEQ ID NO: 5
MGHHHHHHHHHHSSGADSSESALNKAPGYQDFPAYYSDSAHADDQVTHPDVVVLEEPWNGYRYWAVYTPNVMRISIYENPSIV
ASSDGVHWVEPEGLSNPIEPQPPSTRYHNCDADMVYNAEYDAMMAYWNWADDQGGGVGAEVRLRISYDGVHWGVPVTYDEMTR
VWSKPTSDAERQVADGEDDFITAIASPDRYDMLSPTIVYDDFRDVFILWANNTGDVGYQNGQANFVEMRYSDDGITWGEPVRV
NGFLGLDENGQQLAPWHQDVQYVPDLKEFVCISQCFAGRNPDGSVLHLTTSKDGVNWEQVGTKPLLSPGPDGSWDDFQIYRSS
FYYEPGSSAGDGTMRVWYSALQKDTNNKMVADSSGNLTIQAKSEDDRIWRIGYAENSFVEMMRVLLDDPGYTTPALVSGNSLM
LSAETTSLPTGDVMKLETSFAPVDTSDQVVKYTSSDPDVATVDEFGTITGVSVGSARIMAETREGLSDDLEIAVVENPYTLIP
QSNMTATATSVYGGTTEGPASNVLDGNVRTIWHTNYAPKDELPQSITVSFDQPYTVGRFVYTPRQNGTNGIISEYELYAIHQD
GSKDLVASGSDWALDAKDKTVSFAPVEAVGLELKAIAGAGGFGTAAELNVYAYGPIEPAPVYVPVDDRDASLVFTGAWNSDSN
GSFYEGTARYTNEIGASVEFTFVGTAIRWYGQNDVNFGAAEVYVDGVLAGEVNVYGPAAAQQLLFEADGLAYGKHTIRIVCVS
PVVDFDYFSYVGE
Description: Flavonifractor plautii Galactosaminidase
SEQ ID NO: 7
MRGKKFISLTLSTMLCLQLLPTASFAAAPATDTGNAGLIAEGDYAIAGNGVRVTYDADGQTITLYRTEGSGLIQMSKPSPLGG
PVIGGQEVQDFSHISCDVEQSTSGVMGSGQRMTITSQSMSTGLIRTYVLETSDIEEGVVYTATSYEAGASDVEVSWFIGSVYE
LYGAEDRIWSYNGGGEGPMHYYDTLQKIDLTDSGKFSRENKQDDTAASIPVSDIYIADGGITVGDASATRREVHTPVQETSDS
AQVSIGWPGKVIAAGSVIEIGESFAVVHPGDYYNGLRGYKNAMDHLGVIMPAPGDIPDSSYDLRWESWGWGFNWTIDLIIGKL
DELQAAGVKQITLDDGWYTNAGDWALNPEKFPNGASDALRLTDAIHEHGMTALLWWRPCDGGIDSILYQQHPEYFVMDADGRP
ARLPTPGGGTNPSLGYALCPMADGAIASQVDFVNRAMNDWGFDGFKGDYVWSMPECYNPAHNHASPEESTEKQSEIYRVSYEA
MVANDPNVFNLLCNCGTPQDYYSLPYMTQIATADPTSVDQTRRRVKAYKALMGDYFPVTADHNNIWYPSAVGTGSVLIEKRDL
SGTAKEEYEKWLGIADTVQLQKGRFIGDLYSYGFDPYETYVVEKDGVMYYAFYKDGSKYSPTGYPDIELKGLDPNKMYRIVDY
VNDRVVATNLMGDNAVFNTRFSDYLLVKAVEISEPDPEPVDPDYGFTSVDDRDEALIYTGTWHDDNNASFSEGTARYTNSTDA
SVVFSFTGTSIRWYGQRDTNFGTAEVYLDDELKTTVDANGAAEAGVCLFEALDLPAAEHTIKIVCKSGVIDIDRFAYEAATLE
PIYEKVDALSDRITYVGNWEEYHNSEFYMGNAMRTDEAGAYAELTFRGTAVRLYAEMSFNFGTADVYLDGELVENIILYGQEA
TGQLMFERTGLEEGEHTIRLVQNAWNINLDYISYLPEQDQPTPPETTVTVDAMDAQLVYTGVWNDDYHDVFQEGTARYASSAG
ASVEFEFTGSEIRWYGQNDSNFGVASVYIDNEFVQQVNVNGAAAVGKLLFQKADLPAGSHTIRIVCDTPVIDLDYLTYTTNA
Description: Flavonifractor plautii Galactosaminidase (removed signal
peptide Protein seq)
SEQ ID NO: 9
AAPATDTGNAGLIAEGDYAIAGNGVRVTYDADGQTITLYRTEGSGLIQMSKPSPLGGPVIGGQEVQDFSHISCDVEQSTSGVM
GSGQRMTITSQSMSTGLIRTYVLETSDIEEGVVYTATSYEAGASDVEVSWFIGSVYELYGAEDRIWSYNGGGEGPMHYYDTLQ
KIDLTDSGKFSRENKQDDTAASIPVSDIYIADGGITVGDASATRREVHTPVQETSDSAQVSIGWPGKVIAAGSVIEIGESFAV
VHPGDYYNGLRGYKNAMDHLGVIMPAPGDIPDSSYDLRWESWGWGFNWTIDLIIGKLDELQAAGVKQITLDDGWYTNAGDWAL
NPEKFPNGASDALRLTDAIHEHGMTALLWWRPCDGGIDSILYQQHPEYFVMDADGRPARLPTPGGGTNPSLGYALCPMADGAI
ASQVDFVNRAMNDWGFDGFKGDYVWSMPECYNPAHNHASPEESTEKQSEIYRVSYEAMVANDPNVFNLLCNCGTPQDYYSLPY
MTQIATADPTSVDQTRRRVKAYKALMGDYFPVTADHNNIWYPSAVGTGSVLIEKRDLSGTAKEEYEKWLGIADTVQLQKGRFI
GDLYSYGFDPYETYVVEKDGVMYYAFYKDGSKYSPTGYPDIELKGLDPNKMYRIVDYVNDRVVATNLMGDNAVFNTRFSDYLL
VKAVEISEPDPEPVDPDYGFTSVDDRDEALIYTGTWHDDNNASFSEGTARYTNSTDASVVFSFTGTSIRWYGQRDTNFGTAEV
YLDDELKTTVDANGAAEAGVCLFEALDLPAAEHTIKIVCKSGVIDIDRFAYEAATLEPIYEKVDALSDRITYVGNWEEYHNSE
FYMGNAMRTDEAGAYAELTFRGTAVRLYAEMSFNFGTADVYLDGELVENIILYGQEATGQLMFERTGLEEGEHTIRLVQNAWN
INLDYISYLPEQDQPTPPETTVTVDAMDAQLVYTGVWNDDYHDVFQEGTARYASSAGASVEFEFTGSEIRWYGQNDSNFGVAS
VYIDNEFVQQVNVNGAAAVGKLLFQKADLPAGSHTIRIVCDTPVIDLDYLTYTTNA
Description: Flavonifractor plautii Galactosaminidase with HisTag
(pET16a-Protein seq)
SEQ ID NO: 10
MGHHHHHHHHHHSSGAAPATDTGNAGLIAEGDYAIAGNGVRVTYDADGQTITLYRTEGSGLIQMSKPSPLGGPVIGGQEVQDF
SHISCDVEQSTSGVMGSGQRMTITSQSMSTGLIRTYVLETSDIEEGVVYTATSYEAGASDVEVSWFIGSVYELYGAEDRIWSY
NGGGEGPMHYYDTLQKIDLTDSGKFSRENKQDDTAASIPVSDIYIADGGITVGDASATRREVHTPVQETSDSAQVSIGWPGKV
IAAGSVIEIGESFAVVHPGDYYNGLRGYKNAMDHLGVIMPAPGDIPDSSYDLRWESWGWGFNWTIDLIIGKLDELQAAGVKQI
TLDDGWYTNAGDWALNPEKFPNGASDALRLTDAIHEHGMTALLWWRPCDGGIDSILYQQHPEYFVMDADGRPARLPTPGGGTN
PSLGYALCPMADGAIASQVDFVNRAMNDWGFDGFKGDYVWSMPECYNPAHNHASPEESTEKQSETYRVSYEAMVANDPNVFNL
LCNCGTPQDYYSLPYMTQTATADPTSVDQTRRRVKAYKALMGDYFPVTADHNNIWYPSAVGTGSVLIEKRDLSGTAKEEYEKW
LGIADTVQLQKGRFIGDLYSYGFDPYETYVVEKDGVMYYAFYKDGSKYSPTGYPDIELKGLDPNKMYRIVDYVNDRVVATNLM
GDNAVFNTRFSDYLLVKAVEISEPDPEPVDPDYGFTSVDDRDEALIYTGTWHDDNNASFSEGTARYTNSTDASVVFSFTGTSI
RWYGQRDTNFGTAEVYLDDELKTTVDANGAAEAGVCLFEALDLPAAEHTIKIVCKSGVIDIDRFAYEAATLEPIYEKVDALSD
RITYVGNWEEYHNSEFYMGNAMRTDEAGAYAELTFRGTAVRLYAEMSFNFGTADVYLDGELVENTILYGQEATGQLMFERTGL
EEGEHTIRLVQNAWNINLDYISYLPEQDQPTPPETTVTVDAMDAQLVYTGVWNDDYHDVFQEGTARYASSAGASVEFEFTGSE
IRWYGQNDSNFGVASVYIDNEFVQQVNVNGAAAVGKLLFQKADLPAGSHTIRIVCDTPVIDLDYLTYTTNA
Description: Clostridium tertium isolated Protein sequence for identity
099345757.1 - Ct5757 (fusion of Galactosaminidase and GalNAcDeacetylase connected
by an CBM (original Protein sequence)
SEQ ID NO: 12
MKKRILATFITAMCGLGFFSNWTSSNAYNLIDNISVEKLDTDISQANENVFLNGNGIALEVDNRGATCIYLVDENGVKTKATT
SLDTADFSGYPIIGGQKIRDEVIISKNLEENINSILGVGNRLTIISKSSSTNLIRKIVFETSNSNPGAIYSTVSYKAESNDLL
VDSFHENEYTMSLGQGPFLAYQGCADQQGANTIVNVTNGYNHNSGQNNYSVGVPFSYVYNSVGGIGIGDASTSRREFKLPIIG
KDNTVSLGMEWNGQTLKKGAETAIGTSVITTTNGDYYSGLKSYAEVMKDKGISAPASTPDIAYDSRWESWGFEFDFTIEKIVN
KLDELKAMGIKQITLDDGWYTYAGDWKLSPQKFPNGNADMKYLTDEIHKRGMTAILWWRPVDGGINSKLVSEHPEWFIKNSQG
NMVRLPGPGGGNGGTAGYADCPNSEGSIQHHKDFVTVALEEWGFDGFKEDYVWGIPKCYDSSHKHSSLSDTLENQYKFYEAIY
EQSIAINPDTFIELCNCGTPQDFYSTPYVNHAPTADPISRVQTRTRVKAFKAIFGDDFPVTTDHNSVWLPSALGTGSVMITKH
TTLSSSDREQYNKYFGLARDLELAKGEFIGNLYKYGIDPLESYVIRKGEDIYYSFYKDNSSYSGNIEIKGLDSNATYRIEDYV
NNRVIARGVKGPTATINTSFTDNLLVRAIPDDTPAEVTTFDVGNNTILSSTDSGNSKYLNAVSTTLEKTATIDSLSIYIGNNS
ENGKLQIAIYDDNNGKPGTKKAYVEEFVPTKNSWNTKKVVNSVTLPSGQYWLVFQPDNDVLQTKTNPSSMKQSANNNPYNYNI
LPNSFPIGTGYNAYKGDVSFYATFKEASSQAIPQNSWALKYVDSEETTGENGRATNAFDGNNNTIWHTKYSGGNAAPMPHEIQ
IDLRGVYNINQINYLPRQDGGTNGTIKDYEVYLSLDGVNWGQPISKGTFESNSTEKIVKFNETKSRYVKLKALSEINNKQFTT
VADLKVFGWEISKIEKPLQNAETYLNIPTYDGLNQSTHPDVKYFKNGWNGYKYWMIMTPNRTGSSVAENPSILASDDGINWEV
PAGVTNPIAPMPQVGHNCDVDMIYNEATDELWVYWVESDDITKGWVKLIKSKDGVNWSSQQVVVDDNRAKYSTLSPSIIFKDN
KYYMWSVNTGNSGWNNQSNKVELRESSDGVNWSNPTVVNTLAQDGSQIWHVNVEYIPSKNEYWATYPAYKNGTGSDKTELYYA
KSSDGVNWTTYKNPILSKGTSGKWDDMETYRSCFVYDEDTNMIKVWYGAVSQNPQIWKIGFTENDYDKFIEGLTQ
Description: Clostridium tertium 5757 (Ct5757) isolated Protein
sequence with signal peptide removed (identity 099345757.1 - Ct5757)
SEQ ID NO: 14
YNLIDNISVEKLDTDISQANENVFLNGNGIALEVDNRGATCIYLVDENGVKTKATTSLDTADFSGYPIIGGQKIRDEVIISKN
LEENINSILGVGNRLTIISKSSSTNLIRKIVFETSNSNPGAIYSTVSYKAESNDLLVDSFHENEYTMSLGQGPFLAYQGCADQ
QGANTIVNVTNGYNHNSGQNNYSVGVPFSYVYNSVGGIGIGDASTSRREFKLPIIGKDNTVSLGMEWNGQTLKKGAETAIGTS
VITTTNGDYYSGLKSYAEVMKDKGISAPASIPDIAYDSRWESWGFEFDFTIEKIVNKLDELKAMGIKQITLDDGWYTYAGDWK
LSPQKFPNGNADMKYLTDEIHKRGMTAILWWRPVDGGINSKLVSEHPEWFIKNSQGNMVRLPGPGGGNGGTAGYALCPNSEGS
IQHHKDFVTVALEEWGFDGFKEDYVWGIPKCYDSSHKHSSLSDTLENQYKFYEAIYEQSIAINPDTFIELCNCGTPQDFYSTP
YVNHAPTADPISRVQTRTRVKAFKAIFGDDFPVTTDHNSVWLPSALGTGSVMITKHTTLSSSDREQYNKYFGLARDLELAKGE
FIGNLYKYGIDPLESYVIRKGEDIYYSFYKDNSSYSGNIEIKGLDSNATYRIEDYVNNRVIARGVKGPTATINTSFTDNLLVR
AIPDDTPAEVTTFDVGNNTILSSTDSGNSKYLNAVSTTLEKTATIDSLSIYIGNNSENGKLQIAIYDDNNGKPGTKKAYVEEF
VPTKNSWNTKKVVNSVTLPSGQYWLVFQPDNDVLQTKTNPSSMKQSANNNPYNYNILPNSFPIGTGYNAYKGDVSFYATFKEA
SSQAIPQNSWALKYVDSEETTGENGRATNAFDGNNNTIWHTKYSGGNAAPMPHEIQIDLRGVYNINQINYLPRQDGGTNGTIK
DYEVYLSLDGVNWGQPISKGTFESNSTEKIVKFNETKSRYVKLKALSEINNKQFTTVADLKVFGWEISKIEKPLQNAETYLNI
PTYDGLNQSTHPDVKYFKNGWNGYKYWMIMTPNRTGSSVAENPSILASDDGINWEVPAGVTNPIAPMPQVGHNCDVDMIYNEA
TDELWVYWVESDDITKGWVKLIKSKDGVNWSSQQVVVDDNRAKYSTLSPSIIFKDNKYYMWSVNTGNSGWNNQSNKVELRESS
DGVNWSNPTVVNTLAQDGSQIWHVNVEYIPSKNEYWAIYPAYKNGTGSDKTELYYAKSSDGVNWTTYKNPILSKGTSGKWDDM
EIYRSCFVYDEDTNMIKVWYGAVSQNPQIWKIGFTENDYDKFIEGLTQ
Description: Clostridium tertium 5757 (Ct5757) Fusion Protein sequence
expression construct (in pET28a vector) with HisTag and Thrombin Cleavage site
SEQ ID NO: 15
MGSSHHHHHHSSGLVPRGSHYNLIDNISVEKLDTDISQANENVFLNGNGIALEVDNRGATCIYLVDENGVKTKATTSLDTADF
SGYPIIGGQKIRDFVIISKNLEENINSILGVGNRLTIISKSSSTNLIRKIVFETSNSNPGAIYSTVSYKAESNDLLVDSFHEN
EYTMSLGQGPFLAYQGCADQQGANTIVNVTNGYNHNSGQNNYSVGVPFSYVYNSVGGIGIGDASTSRREFKLPIIGKDNTVSL
GMEWNGQTLKKGAETAIGTSVITTTNGDYYSGLKSYAEVMKDKGISAPASIPDIAYDSRWESWGFEFDFTIEKIVNKLDELKA
MGIKQITLDDGWYTYAGDWKLSPQKFPNGNADMKYLTDEIHKRGMTAILWWRPVDGGINSKLVSEHPEWFIKNSQGNMVRLPG
PGGGNGGTAGYALCPNSEGSIQHHKDFVTVALEEWGFDGFKEDYVWGIPKCYDSSHKHSSLSDTLENQYKFYEAIYEQSIAIN
PDTFIELCNCGTPQDFYSTPYVNHAPTADPISRVQTRTRVKAFKAIFGDDFPVTTDHNSVWLPSALGTGSVMITKHTTLSSSD
REQYNKYFGLARDLELAKGEFIGNLYKYGIDPLESYVIRKGEDIYYSFYKDNSSYSGNIEIKGLDSNATYRIEDYVNNRVIAR
GVKGPTATINTSFTDNLLVRAIPDDTPAEVTTFDVGNNTILSSTDSGNSKYLNAVSTTLEKTATIDSLSIYIGNNSENGKLQI
AIYDDNNGKPGTKKAYVEEFVPTKNSWNTKKVVNSVTLPSGQYWLVFQPDNDVLQTKTNPSSMKQSANNNPYNYNILPNSFPI
GTGYNAYKGDVSFYATFKEASSQAIPQNSWALKYVDSEETTGENGRATNAFDGNNNTIWHTKYSGGNAAPMPHEIQIDLRGVY
NINQINYLPRQDGGTNGTIKDYEVYLSLDGVNWGQPISKGTFESNSTEKIVKFNETKSRYVKLKALSEINNKQFTTVADLKVF
GWEISKIEKPLQNAETYLNIPTYDGLNQSTHPDVKYFKNGWNGYKYWMIMTPNRTGSSVAENPSILASDDGINWEVPAGVTNP
IAPMPQVGHNCDVDMIYNEATDELWVYWVESDDITKGWVKLIKSKDGVNWSSQQVVVDDNRAKYSTLSPSIIFKDNKYYMWSV
NTGNSGWNNQSNKVELRESSDGVNWSNPTVVNTLAQDGSQIWHVNVEYIPSKNEYWAIYPAYKNGTGSDKTELYYAKSSDGVN
WTTYKNPILSKGTSGKWDDMEIYRSCFVYDEDTNMIKVWYGAVSQNPQIWKIGFTENDYDKFIEGLTQ
Description: Clostridium tertium 5757 (Ct5757) GalNAcDeacetylase
Protein sequence expression construct (in pET28a vector) with HisTag and
Thrombin Cleavage site
SEQ ID NO: 17
MGSSHHHHHHSSGLVPRGSHSGQYWLVFQPDNDVLQTKTNPSSMKQSANNNPYNYNILPNSFPIGTGYNAYKGDVSFYATFKE
ASSQAIPQNSWALKYVDSEETTGENGRATNAFDGNNNTIWHTKYSGGNAAPMPHEIQIDLRGVYNINQINYLPRQDGGTNGTI
KDYEVYLSLDGVNWGQPISKGTFESNSTEKIVKFNETKSRYVKLKALSEINNKUTTVADLKVEGWEISKIEKPLQNAETYLN
IPTYDGLNQSTHPDVKYEKNGWNGYKYWMIMTPNRTGSSVAENPSILASDDGINWEVPAGVTNPIAPMPQVGHNCDVDMIYNE
ATDELWVYWVESDDITKGWVKLIKSKDGVNWSSQQVVVDDNRAKYSTLSPSIIFKDNKYYMWSVNTGNSGWNNQSNKVELRES
SDGVNWSNPTVVNTLAQDGSQIWHVNVEYIPSKNEYWAIYPAYKNGTGSDKTELYYAKSSDGVNWTTYKNPILSKGTSGKWDD
MEIYRSCFVYDEDTNMIKVWYGAVSQNPQIWKIGFTENDYDKFIEGLTQ
Description: Clostridium tertium 5757 (Ct5757) Protein sequence
Calactosaminidase_expression construct (in pET28a vector) with HisTag and
Thrombin Cleavage site
SEQ ID NO: 19
MGSSHHHHHHSSGLVPRGSHYNLIDNISVEKLDTDISQANENVELNGNGIALEVDNRGATCIYLVDENGVKTKATTSLDTADF
SGYPIIGGQKIRDEVIISKNLEENINSILGVGNRLTIISKSSSTNLIRKIVFETSNSNPGAIYSTVSYKAESNDLLVDSFHEN
EYTMSLGQGPFLAYQGCADQQGANTIVNVTNGYNHNSGQNNYSVGVPFSYVYNSVGGIGIGDASTSRREFKLPIIGKDNTVSL
GMEWNGQTLKKGAETAIGTSVITTTNGDYYSGLKSYAEVMKDKGISAPASIPDIAYDSRWESWGFEEDFTIEKIVNKLDELKA
MGIKQITLDDGWYTYAGDWKLSPQKFPNGNADMKYLTDEIHKRGMTAILWWRPVDGGINSKLVSEHPEWFIKNSQGNMVRLPG
PGGGNGGTAGYALCPNSEGSIQHHKDEVTVALEEWGEDGEKEDYVWGIPKCYDSSHKHSSLSDTLENQYKEYEAIYEQSIAIN
PDTFIELCNCGTPQDFYSTPYVNHAPTADPISRVQTRTRVKAFKAIFGDDFPVTTDHNSVWLPSALGTGSVMITKHTTLSSSD
REQYNKYFGLARDLELAKGEFIGNLYKYGIDPLESYVIRKGEDIYYSFYKDNSSYSGNIEIKGLDSNATYRIEDYVNNRVIAR
GVKGPTATINTSFTDNLLVRAIPDDTPAEVTTFDVGNNTILSSTDSGNSKYLNAVSTTLEKTATIDSLSIYIGNNSENGKLQI
AIYDDNNGKPGTKKAYVEEFVPTKNSWNTKKVVNSVTLPSGQYWLVFQPDNDVLQTKTNPSSMKQSANNNPYNYNILPNSFPI
GTGYNAYKGDVSFYATEKEASSQAIPQNSWALKYVDSEETTGENGRATNAFDGNNNTIWHTKYSGGNAAPMPHEIQIDLRGVY
NINQINYLPRQDGGTNGTIKDYEVYLSLDGVNWGQPISKGTFESNSTEKIVKFNETKSRYVKLKALSEINNKUTTVADLKVF
GWEISKIEK
Description: Robinsoniella peoriensis Rp1021 Galactosaminidase Protein
expression construct (in pET28a vector) with HisTag and Thrombin Cleavage site
SEQ ID NO: 21
MGSSHHHHHHSSGLVPRGSHGNGLEVKASPREVAQITGNGVSVTFFQEDGTVQLSCIEDDGNTAFMTRNSEVSYPVVGGEEVT
DFSDFQCEVQENVTGAAGAGSRMTITSISSGRGIQRSVVIETVDEVKGLLHISSSYRAEEEVDADEFIDSRFSLDNPSDTVWS
YNGGGEGAQSRYDTLQKIDLSDGESFYRENLQNQTAAGIPVADIYGKDGGITVGDASVTRRQLSTPVNERNGTAYVSVKHPGA
VITQRETEISQSFVNVHRGDYYSGLRGYADGMKQIGETTLSREQIPESSYDLRWESWGWEEDWTVELIINKLDELKEMGIKQI
TLDDGWYNAAGEWGLNNWKLPNGALDMRHLTDAIHERGMTAVLWWRPCDGGREDSALFKEHPEYFIKNQDGSFGKLAGPGQWN
SFLGSCGYALCPLSEGAVQSQVDFINRAMNEWGEDGEKSDYVWSLPKCYSQDHHHEYPEESTEQQAVEYRAVYEAMTDNDPNA
FHLLCNCGTPQDYYSLPYVTQVPTADPTSVDQTRRRVKAYKALCGDYFPVTTDHNEVWYPSTIGTGAILIEKRDLSGWEEEEY
AKWLKIAQENQLHKGTFIGDLYSYGYDPYETYTVYKDGIMYYAFYKDGNRYRPSGNPDIELKGLEDGKLYRIVDYVNNQVVAT
NVTSSNAVESYPFSDYLLVKAVEISEPDTDGPGPVPDPEGAVTVEENDPELVYTGDWVREENDGYHGGGARYTKEAEASVELA
FYGTGAAWYGQHDVNFGSARIYIDGTYVKTVSCMGEPGINIKLFEISGLDLASHRIKIECETPVIDIDRLTYIKGEEVPAKVM
TADLRALTVIANQYDMNSFADGNYKDQLGVSLVRANQLLAADDVTQGAVNEEQKYLLNAMLKIRKKVDKSWIGLPGPIPQDIQ
TENISRDNLAKVISYTGQLDRDEIIPAIKEQLNDSYDKAVSIAERQDASQPEIDRAWAELMNAVQYSSYIRGSKEELLSLLDE
YGKVDTTVYKDAALFIESLEAAKKVYQDENAMDGEISDCIKQLRDAKDQLQLKDPVDPPKPDPDPDPKPDPTPDPGPDPKPDP
TPDPTPDPKPNPTPTPDPTPEPALKKPEQVSGLKSKAETDYLTVSWKKLNNAESYKVYIYKSGKWRLAGKTTKTSIKIKKLVS
GTKYTVKVAAVNKAGQGKYSSQVYTAAKPKKVKLKSVSRYRTSKVKLNYGKVKAGGYEIWMKNGKGSYKKAATSTKTTAIKSG
LKKGKTYYFKVRAYVKNKNQVIYGSFSNIKKYKMVL
Description: Ruthenibacterium lactatiformans R18755 GalNAcDeacetylase
protein Sequence expression construct (in pET28a vector) with HisTag and
Thrombin Cleavage site
SEQ ID NO: 23
MGSSHHHHHHSSGLVPRGSHEETDLLVNGGFETGDSTGWNWFNNAVVDSAAPHSGNYCAKVAKNSSYEQVVTVSPDTKYVLTG
WAKSEGSSVMTLGVKNYGGQETFSATLSADYQQLAVTFTTGPNAQTATIYGYRQNSGSGAGYFDDVELTAVQDFAPYQPLANA
IAPQAIPTYDGANQPTHPSVVKFEQPWNGYLYWMAMTPYPFNDGSYENPSIVASNDGENWIVPEGVSNPLAGTPSPGHNCDVD
LVYVPASDELRMYYVEADDIISSRVKMISSRDGVHWSEPQVVMQDLVRKYSILSPSIEILPDGTYMMWYVDTGNAGWNSQNNQ
VKYRTSADGIKWSGAVTCTDFVQPGYQIWHIDVHYDTSSGAYYAVYPAYPNGTDCDHCNLFFAVNRTGKQWETFSRPILKPST
EGGWDDFCIYRSSMLIDDGMLKVWYGAKKQEDSSWHTGLTMRDFSEFMKILER
Description: Robinsoniella peoriensis Rp3671 GalNAcDeacetylase
protein_expression construct (in pET28a vector) with HisTag and Thrombin
Cleavage site
SEQ ID NO: 25
MGSSHHHHHHSSGLVPRGSHSPLSAAAESGTGTRLVKGQTGYLTEEQAIRNQEQTTEEREQKLTGEETAEVLMEGTKDSGIVQ
TEEVQTKEMQTEDAQTEEVQTEEMQTEDAQTKEVQTEEMQTEDAQTEEVQTKEEPAEETHMKEIQTQGTKKASDRNGKARVTE
ILEDAQDPANRIVYLSDLQWKSENHTVDSELPTRKDKSFGGGKITLKVDGTVTEFDKGIGTQTDSTIVYDLEGKGYTKFETYV
GVDYSQKENIPGEVCDVKFRVKIDDKIVSETGVLDPLSNAVKISVNIPDTAKTLTLYADKVTETWSDHANWADAKFYQALPEP
ENVAFKKTVVTRKTSDNSEAPVNPDSAVNSSKAVDGVIDSSSYFDFGDQANSGAVRESLYMEVDLKGSYLLSDIQLWRYWKDG
RTYAATAIVVAEDENFENAAVIYNSDTTGEIHHLGAGSDMLYAETESGKTFPVPENTKARYIRVYTYGVNGTSGVTNHIVELK
VNAYVFGDEILPEKPDDSKIFPNAVNPLKLQGPGTNDQVTHPDVTVFDEPWNGYKYWMAYTPNKPGSSYFENPCIAASNDGVN
WEFPAQNPVQPRYDSEIENQNEHNCDTDIVYDPVNDRLIMYWEWAQDEAVNGKTHRSEIRYRVSYDGINWGVEDKTGVLMTGP
TDHGCAIATEGERYSDLSPTVVYDKTEKIYKMWANDAGDVGYENKQNNKVWYRTSQDGISNWSDKTYVENFLGVNEDGLQMYP
WHQDIQWVEEFQEYWALQQAFPAGSGPDNSSLRFSKSKDGLHWEPVSEKALITVGAPGTWDAGQIYRSTFWYEPGGAKGNGTF
HIWYAALAEGQSHWDIGYTSANYADAMYKLTGSRPEVEKRIEVNNENPLLIMPLYGKSYSESGSTLDWGDDLVSRWKQVPEDL
KENAVIEIHLGGKIGLNESDSHTAKAFYEQQLAIAQENNIPVMMVVATAGQQNYWTGTANLDAEWIDRMFKQHSVLKGIMSTE
NYWTDYNKVATMGADYLRVAAENGGYFVWSEHQEGVIENVIANEKFNEALKLYGNNFIFTWKNTPAGTNSNAGTASYMQGLWL
TGICAQWGGLADTWKWYEKGFGKLFDGQYSYNPGGEEARPVATEPEALLGIEMMSIYTNGGCVYNFEHPAYVYGSYNQNSPCF
ENVIAEFMRYAIKNPAPGKEEVLADTKAVFYGKLSSLKSAGNLLQKGLNWEDATLPTQTTGRYGLIPAVPEAVDEKTVKAVFG
DIEILNQSSAQLANKDAKKAYFEEKYPEQYTGTAFGQLLNDTWYLYNSNVNVDGVQNAKLPLEGNKSVDITMTPHTYVILDDQ
DGELQIKLNNYRVDKDSIWEGYGTTVTDRWDTDHNTKLQDWIRDEYIPNPDDDTFRDTTFELVGLESEPEVNVTNGLKDQYQE
PVVEYDAAAGTAMITVSGNGWVDLTIDTNTAEVPQVDKAKLNSKIAEAKGIRQGNYTDESYKALQEEIGKSQAVSNKTDATQE
EVNAQLSRLESAIARLKEKPAVVSKTAINAKIAEAKGIRQGNYTDESYKALQNAIVKAQELSNKTDATQQQVNDLVSALTNAI
KNLKIDADKLAAESAKKVAAVKVAVKAVSYKSKEIKLSWKTVADADGYVIRVKTGKKWSTEKTIKNNRIITYTYKKGTPGKKY
VFEVKAFKKVNGKTTYSKYKTATKKVVPQTVTAKAKASKNNVVVKWNKVSGASGYVVMKKKGKTWVKAAQVNAKKLYFTDKKV
KKGKVYSYKVKAYKVYKGKKVYGSYSKSVNVKTKS
Description: Robinsoniella peoriensis Rp3672 GalNAcDeacetylase
protein_expression construct (in pET28a vector) with HisTag and Thrombin
Cleavage site
SEQ ID NO: 27
MGSSHHHHHHSSGLVPRGSHAETATEENAALEKTVTLHKSDGTELPEDYRNPQRPATMAVDGIIDDTGEYNYCDFGKDGDKAA
LYMQVDLGGLYDLSRVNMWRYWKDSRTYDATVITTSESGDFTDEAVIYNSDRSNVHGFGAGGDERYAETASGHEFPVPDGTKA
QAVRVYVFGSQNGTTNHINELQVWGTPHTENPDVNSYQVTIPQGNGYQVIPYENDPTTVEEGGSFRFQVLIDSDNGYSATSAV
KANGVSLEAVDSVYTIENITEDQVITIEGVHKAQYEVKFPENPQGYSVEIQNEGSTTVDYNGSVSFKLIIDEAYNESVPVVKA
NGGAALGKDELGVYTIANIQDDITVTVEGIQENTVVKTKTMYLSDMDWKSAANAVGATGEKDTPTKDLNHLQQQMKLLVNGAE
KSFDKGIGVQTDSSIVYDLEDKGYTSFHTLAGVDYSAMEYVDGEGCDIQFKVYLDDVVVFDSGVVDASDEAQEVNVAITSENK
ELKLEAKMVKEPYNDWGNWADASFEMAYPEPSNVALNKTVTVKKTADNSDSEVNSSRPGSMAVDGIIGPTSDSNYCDFGQDGD
NTSRYLQVDLGDVYELTQINMFRYWADGRVYNGTVIAVSENADFSNPTFIYNSDKADKHGLGAGSDDTYGETQSGKLFEVPAG
TMGQYVRVYMAGSNKGTTNHIAELQVMGYNFNTEPKPYEANAFENAEVYLDMPTHFQDLDSNKNDDGSLKHIGGQVTHPDIQV
FDQPWNGYKYWMIYTPNTMITSQYENPYIVASEDGQTWVEPEGISNPIEPEPPSTRFHNCDADLLYDSVNDRLLAYWNWADDG
GGIDDELKDQNCQIRLRISYDGINWGVPYDKDGNIATTADTVVRMETGDKDFIPAISEKDRYGMLSPTFTYDDFRGIYTMWAQ
NSGDAGYNQSGKFIEMRWSEDGINWSEPQKVNNFLGKDENGRQLWPWHQDIQYIPELQEYWGLSQCFSTSNPDGSVLYLTKSR
DGVNWEQAGTQPVLRAGKSGTWDDFQIYRSTFYYDNQSDSPTGGKFRIWYSALQANTSGKTVLAPDGTVSLQVGSQDTRIWRI
GYTENDYMEVMKALTQNKNYEEPELVDAVSLNLSMDKTSISVGEEATVSTAFVPENATDRIVKYTSQDPEIAVIDPTGIVTGV
KDGTTTIVAETKSGAKGELSVTVGELQRGEIRFEVSNDHPMYLENYYWSDDAPKKDGLDANKNYYGDERVDSPVMLYNTVPEE
LKDNTVILLIAERSLNSTDAVRDWIKKNVELCNENKIPCAVQIANGETNVNTTIPLSFWNELATNNEYLVGFNAAEMYNRFAG
DNRSYVMDMIRLGVSHGVCMMWTDTNIFGTNGVLYDWLTQDEKLSGLMREYKEYISLMTKESYGSEAANTDALFKGLWMTDYC
ENWGIASDWWHWQLDSNGALFDAGSGGDAWKQCLTWPENMYTQDVVRAVSQGATCFKSEAQWYSNATKGMRTPTYQYSMIPFL
EKLVSKEVKIPTKEEMLERTKAIVVGAENWNNFNYNTTYSNLYPSTGQYGIVPYVPSNCPEEELAGYDLVVRENLGKAGLKSA
LDTVYPVQKSEGTAYCETFGDTWYWMNSSEDKNVSQYTEFTTAINGAESVKIAGEPHVFGIIKENPGSLNVYLSNYRLDKTEL
WDGTIPGGLSDQGCYNYVWQMCERMKNGTGLDTQLRDTVITVKNAVEPKVNFVTESPADRSFAEDNYVRPYKYTVAQKEGTTD
EWVITVSHNGIVEFNIVTGDEKVPATSVELSTDKVDVIRNRTAVVKATVLPQNAGNKQLTWTIADPEIASVDNKGTVTGLKEG
KTVLRAAISGSVYKECEVNVIDRKVTEVNLNKTELSLSAGDSAKLEASIAPEDPSDSSITWTSTNENVATVASNGTVTAHKAG
VAQIIAQSAYQAKGIATVTVNYAASVKLDRTGMTATANSEQSKSGGEGPASNVLDGKQDTMWHTSWTDKPELHPHWIKIDLNG
TKTINKFAYTPRTGASNGTIYNYVLIITDLEGNEKQVAKGVWAANADVKYAEFDAVEATAIKLQVDGNDDKASKGGYGSAAEI
NIFEVAQKPSANELAENIKVIAPVKAEDTKVSIPVITGFDIVISNSSNPDVIGIDGSITRPENDTVVTLTLKVKETDAKSVKA
AGTEATTNVDVLVTGTKTSDVEAESVTLDQTSADLTVGGELLLNAVVKPDIATNKAVTWSSDKPGTATVENGRVKALAAGEAR
ITAATANGKTADCVINVKEKEEPEVILPAEVRLNIPSAEFTVGDQIQLTASVLPANAADKTITWKSDKPEVATVANGWVKGIA
AGTAKITATSVNGKTAVCVITVKAQPQNLPTGVSLNKKTASVKLNKTLTLSAVVQPSNADNKTVKWTSDNTYVATVENGVVKA
VNAGTARITAATVNGHKATCTITVPGTKISKAKVSLASSKTHTGKAIKPSVKVTYGKNTLKKNTDYTVSYKNNINPGTASVTI
TGKGKYYGTINKTFAIKAAEGKTYTVGKGKYKVTDASAKNKTVTFMAPVKKTYSSFSVPSKVKIGNDTYKVTAVAKNAFKKNT
KLTKLTIGSNVKTIGSYAFYGASQLKTLTLKTTGLNSVGKNAFKKTNAKLTVKVPKSKLADYKKLLKGKGLSGKAKIQK
Description: Robinsoniella peoriensis Rp3671 GalNAcDeacetylase Protein
Rp3671_expression construct (in pET28a vector) with HisTag and Thrombin Cleavage
site
SEQ ID NO: 29
MGSSHHHHHHSSGLVPRGSHSPLSAAAESGTGTRLVKGQTGYLTEEQAIRNQEQTTEEREQKLTGEETAEVLMEGTKDSGIVQ
TEEVQTKEMQTEDAQTEEVQTEEMQTEDAQTKEVQTEEMQTEDAQTEEVQTKEEPAEETHMKEIQTQGTKKASDRNGKARVTE
ILEDAQDPANRIVYLSDLQWKSENHTVDSELPTRKDKSFGGGKITLKVDGTVTEFDKGIGTQTDSTIVYDLEGKGYTKFETYV
GVDYSQKENIPGEVCDVKFRVKIDDKIVSETGVLDPLSNAVKISVNIPDTAKTLTLYADKVTETWSDHANWADAKFYQALPEP
ENVAFKKTVVTRKTSDNSEAPVNPDSAVNSSKAVDGVIDSSSYFDFGDQANSGAVRESLYMEVDLKGSYLLSDIQLWRYWKDG
RTYAATAIVVAEDENFENAAVIYNSDTTGEIHHLGAGSDMLYAETESGKTFPVPENTKARYIRVYTYGVNGTSGVTNHIVELK
VNAYVFGDEILPEKPDDSKIFPNAVNPLKLQGPGTNDQVTHPDVTVFDEPWNGYKYWMAYTPNKPGSSYFENPCIAASNDGVN
WEFPAQNPVQPRYDSEIENQNEHNCDTDIVYDPVNDRLIMYWEWAQDEAVNGKTHRSEIRYRVSYDGINWGVEDKTGVLMTGP
TDHGCAIATEGERYSDLSPTVVYDKTEKIYKMWANDAGDVGYENKQNNKVWYRTSQDGISNWSDKTYVENFLGVNEDGLQMYP
WHQDIQWVEEFQEYWALQQAFPAGSGPDNSSLRFSKSKDGLHWEPVSEKALITVGAPGTWDAGQIYRSTFWYEPGGAKGNGTF
HIWYAALAEGQSHWDIGYTSANYADAMYKLTGSR
Description: Robinsoniella peoriensis Rp3672_GalNAcDeacetylase_protein
expression construct (in pET28a vector)with HisTag and Thrombin Cleavage site
SEQ ID NO: 31
MGSSHHHHHHSSGLVPRGSHAETATEENAALEKTVTLHKSDGTELPEDYRNPQRPATMAVDGIIDDTGEYNYCDFGKDGDKAA
LYMQVDLGGLYDLSRVNMWRYWKDSRTYDATVITTSESGDFTDEAVIYNSDRSNVHGFGAGGDERYAETASGHEFPVPDGTKA
QAVRVYVFGSQNGTTNHINELQVWGTPHTENPDVNSYQVTIPQGNGYQVIPYENDPTTVEEGGSFRFQVLIDSDNGYSATSAV
KANGVSLEAVDSVYTIENITEDQVITIEGVHKAQYEVKFPENPQGYSVEIQNEGSTTVDYNGSVSFKLIIDEAYNESVPVVKA
NGGAALGKDELGVYTIANIQDDITVTVEGIQENTVVKTKTMYLSDMDWKSAANAVGATGEKDTPTKDLNHLQQQMKLLVNGAE
KSFDKGIGVQTDSSIVYDLEDKGYTSFHTLAGVDYSAMEYVDGEGCDIQFKVYLDDVVVFDSGVVDASDEAQEVNVAITSENK
ELKLEAKMVKEPYNDWGNWADASFEMAYPEPSNVALNKTVTVKKTADNSDSEVNSSRPGSMAVDGIIGPTSDSNYCDFGQDGD
NTSRYLQVDLGDVYELTQINMFRYWADGRVYNGTVIAVSENADFSNPTFIYNSDKADKHGLGAGSDDTYGETQSGKLFEVPAG
TMGQYVRVYMAGSNKGTTNHIAELQVMGYNFNTEPKPYEANAFENAEVYLDMPTHFQDLDSNKNDDGSLKHIGGQVTHPDIQV
FDQPWNGYKYWMIYTPNTMITSQYENPYIVASEDGQTWVEPEGISNPIEPEPPSTRFHNCDADLLYDSVNDRLLAYWNWADDG
GGIDDELKDQNCQIRLRISYDGINWGVPYDKDGNIATTADTVVRMETGDKDFIPAISEKDRYGMLSPTFTYDDFRGIYTMWAQ
NSGDAGYNQSGKFIEMRWSEDGINWSEPQKVNNFLGKDENGRQLWPWHQDIQYIPELQEYWGLSQCFSTSNPDGSVLYLTKSR
DGVNWEQAGTQPVLRAGKSGTWDDFQIYRSTFYYDNQSDSPTGGKFRIWYSALQANTSGKTVLAPDGTVSLQVGSQDTRIWRI
GYTENDYMEVMKALTQNKNYEE
Description: Clostridium tertium 5757 (Ct5757) GalNAcDeacetylase
Protein sequence
SEQ ID NO: 32
HSGQYWLVFQPDNDVLQTKTNPSSMKQSANNNPYNYNILPNSFPIGTGYNAYKGDVSFYATFKEASSQAIPQNSWALKYVDSE
ETTGENGRATNAFDGNNNTIWHTKYSGGNAAPMPHEIQIDLRGVYNINQINYLPRQDGGTNGTIKDYEVYLSLDGVNWGQPIS
KGTFESNSTEKIVKFNETKSRYVKLKALSEINNKQFTTVADLKVFGWEISKIEKPLQNAETYLNIPTYDGLNQSTHPDVKYFK
NGWNGYKYWMIMTPNRTGSSVAENPSILASDDGINWEVPAGVTNPIAPMPQVGHNCDVDMIYNEATDELWVYWVESDDITKGW
VKLIKSKDGVNWSSQQVVVDDNRAKYSTLSPSIIFKDNKYYMWSVNTGNSGWNNQSNKVELRESSDGVNWSNPTVVNTLAQDG
SQIWHVNVEYIPSKNEYWAIYPAYKNGTGSDKTELYYAKSSDGVNWTTYKNPILSKGTSGKWDDMEIYRSCFVYDEDTNMIKV
WYGAVSQNPQIWKIGFTENDYDKFIEGLTQ
Description: Ruthenibacterium lactatiformans R18755 GalNAcDeacetylase
protein sequence
SEQ ID NO: 33
HEETDLLVNGGFETGDSTGWNWFNNAVVDSAAPHSGNYCAKVAKNSSYEQVVTVSPDTKYVLTGWAKSEGSSVMTLGVKNYGG
QETFSATLSADYQQLAVTFTTGPNAQTATIYGYRQNSGSGAGYFDDVELTAVQDFAPYQPLANAIAPQAIPTYDGANQPTHPS
VVKFEQPWNGYLYWMAMTPYPFNDGSYENPSIVASNDGENWIVPEGVSNPLAGTPSPGHNCDVDLVYVPASDELRMYYVEADD
IISSRVKMISSRDGVHWSEPQVVMQDLVRKYSILSPSIEILPDGTYMMWYVDTGNAGWNSQNNQVKYRTSADGIKWSGAVTCT
DFVQPGYQIWHIDVHYDTSSGAYYAVYPAYPNGTDCDHCNLFFAVNRTGKQWETFSRPILKPSTEGGWDDFCIYRSSMLIDDG
MLKVWYGAKKQEDSSWHTGLTMRDFSEFMKILER
Description: Robinsoniella peoriensis Rp3671 GalNAcDeacetylase Protein
SEQ ID NO: 34
HSPLSAAAESGTGTRLVKGQTGYLTEEQAIRNQEQTTEEREQKLTGEETAEVLMEGTKDSGIVQTEEVQTKEMQTEDAQTEEV
QTEEMQTEDAQTKEVQTEEMQTEDAQTEEVQTKEEPAEETHMKEIQTQGTKKASDRNGKARVTEILEDAQDPANRIVYLSDLQ
WKSENHTVDSELPTRKDKSEGGGKITLKVDGTVTEFDKGIGTQTDSTIVYDLEGKGYTKFETYVGVDYSQKENIPGEVCDVKF
RVKIDDKIVSETGVLDPLSNAVKISVNIPDTAKTLTLYADKVTETWSDHANWADAKFYQALPEPENVAFKKTVVTRKTSDNSE
APVNPDSAVNSSKAVDGVIDSSSYFDFGDQANSGAVRESLYMEVDLKGSYLLSDIQLWRYWKDGRTYAATAIVVAEDENFENA
AVIYNSDTTGEIHHLGAGSDMLYAETESGKTFPVPENTKARYIRVYTYGVNGTSGVTNHIVELKVNAYVFGDEILPEKPDDSK
IFPNAVNPLKLQGPGTNDQVTHPDVTVFDEPWNGYKYWMAYTPNKPGSSYFENPCIAASNDGVNWEFPAQNPVQPRYDSEIEN
QNEHNCDTDIVYDPVNDRLIMYWEWAQDEAVNGKTHRSEIRYRVSYDGINWGVEDKTGVLMTGPTDHGCAIATEGERYSDLSP
TVVYDKTEKIYKMWANDAGDVGYENKQNNKVWYRTSQDGISNWSDKTYVENFLGVNEDGLQMYPWHQDIQWVEEFQEYWALQQ
AFPAGSGPDNSSLRFSKSKDGLHWEPVSEKALITVGAPGTWDAGQIYRSTFWYEPGGAKGNGTFHIWYAALAEGQSHWDIGYT
SANYADAMYKLTGSR
Description: Robinsoniella peoriensis Rp3672_GalNAcDeacetylase_protein
SEQ ID NO: 35
HAETATEENAALEKTVTLHKSDGTELPEDYRNPQRPATMAVDGIIDDTGEYNYCDFGKDGDKAALYMQVDLGGLYDLSRVNMW
RYWKDSRTYDATVITTSESGDFTDEAVIYNSDRSNVHGEGAGGDERYAETASGHEFPVPDGTKAQAVRVYVEGSQNGTTNHIN
ELQVWGTPHTENPDVNSYQVTIPQGNGYQVIPYENDPTTVEEGGSFRFQVLIDSDNGYSATSAVKANGVSLEAVDSVYTIENI
TEDQVITIEGVHKAQYEVKFPENPQGYSVEIQNEGSTTVDYNGSVSFKLIIDEAYNESVPVVKANGGAALGKDELGVYTIANI
QDDITVTVEGIQENTVVKTKTMYLSDMDWKSAANAVGATGEKDTPTKDLNHLQQQMKLLVNGAEKSFDKGIGVQTDSSIVYDL
EDKGYTSFHTLAGVDYSAMEYVDGEGCDIQFKVYLDDVVVFDSGVVDASDEAQEVNVAITSENKELKLEAKMVKEPYNDWGNW
ADASFEMAYPEPSNVALNKTVTVKKTADNSDSEVNSSRPGSMAVDGIIGPTSDSNYCDFGQDGDNTSRYLQVDLGDVYELTQI
NMFRYWADGRVYNGTVIAVSENADFSNPTFIYNSDKADKHGLGAGSDDTYGETQSGKLFEVPAGTMGQYVRVYMAGSNKGTTN
HIAELQVMGYNFNTEPKPYEANAFENAEVYLDMPTHFQDLDSNKNDDGSLKHIGGQVTHPDIQVFDQPWNGYKYWMIYTPNTM
ITSQYENPYIVASEDGQTWVEPEGISNPIEPEPPSTRFHNCDADLLYDSVNDRLLAYWNWADDGGGIDDELKDQNCQIRLRIS
YDGINWGVPYDKDGNIATTADTVVRMETGDKDFIPAISEKDRYGMLSPTFTYDDFRGIYTMWAQNSGDAGYNQSGKFIEMRWS
EDGINWSEPQKVNNFLGKDENGRQLWPWHQDIQYIPELQEYWGLSQCFSTSNPDGSVLYLTKSRDGVNWEQAGTQPVLRAGKS
GTWDDFQIYRSTFYYDNQSDSPTGGKFRIWYSALQANTSGKTVLAPDGTVSLQVGSQDTRIWRIGYTENDYMEVMKALTQNKN
YEE
Description: Clostridium tertium 5757 (Ct5757) Galactosaminidase
Protein sequence
SEQ ID NO: 36
HYNLIDNISVEKLDTDISQANENVFLNGNGIALEVDNRGATCIYLVDENGVKTKATTSLDTADFSGYPIIGGQKIRDFVIISK
NLEENINSILGVGNRLTIISKSSSTNLIRKIVFETSNSNPGAIYSTVSYKAESNDLLVDSFHENEYTMSLGQGPFLAYQGCAD
QQGANTIVNVTNGYNHNSGQNNYSVGVPFSYVYNSVGGIGIGDASTSRREFKLPIIGKDNTVSLGMEWNGQTLKKGAETAIGT
SVITTTNGDYYSGLKSYAEVMKDKGISAPASIPDIAYDSRWESWGFEFDFTIEKIVNKLDELKAMGIKQITLDDGWYTYAGDW
KLSPQKFPNGNADMKYLTDEIHKRGMTAILWWRPVDGGINSKLVSEHPEWFIKNSQGNMVRLPGPGGGNGGTAGYALCPNSEG
SIQHHKDFVTVALEEWGFDGFKEDYVWGIPKCYDSSHKHSSLSDTLENQYKFYEAIYEQSIAINPDTFIELCNCGTPQDFYST
PYVNHAPTADPISRVQTRTRVKAFKAIFGDDFPVTTDHNSVWLPSALGTGSVMITKHTTLSSSDREQYNKYFGLARDLELAKG
EFIGNLYKYGIDPLESYVIRKGEDIYYSFYKDNSSYSGNIEIKGLDSNATYRIEDYVNNRVIARGVKGPTATINTSFTDNLLV
RAIPDDTPAEVTTFDVGNNTILSSTDSGNSKYLNAVSTTLEKTATIDSLSIYIGNNSENGKLQIAIYDDNNGKPGTKKAYVEE
FVPTKNSWNTKKVVNSVTLPSGQYWLVFQPDNDVLQTKTNPSSMKQSANNNPYNYNILPNSFPIGTGYNAYKGDVSFYATFKE
ASSQAIPQNSWALKYVDSEETTGENGRATNAFDGNNNTIWHTKYSGGNAAPMPHEIQIDLRGVYNINQINYLPRQDGGTNGTI
KDYEVYLSLDGVNWGQPISKGTFESNSTEKIVKFNETKSRYVKLKALSEINNKQFTTVADLKVFGWEISKIEK
Description: Robinsoniella peoriensis Rp1021 Galactosaminidase Protein
Sequence
SEQ ID NO: 37
HGNGLEVKASPREVAQITGNGVSVTFFQEDGTVQLSCIEDDGNTAFMTRNSEVSYPVVGGEEVTDFSDFQCEVQENVTGAAGA
GSRMTITSISSGRGIQRSVVIETVDEVKGLLHISSSYRAEEEVDADEFIDSRFSLDNPSDTVWSYNGGGEGAQSRYDTLQKID
LSDGESFYRENLQNQTAAGIPVADIYGKDGGITVGDASVTRRQLSTPVNERNGTAYVSVKHPGAVITQRETEISQSFVNVHRG
DYYSGLRGYADGMKQIGFTTLSREQIPESSYDLRWESWGWEFDWTVELIINKLDELKEMGIKQITLDDGWYNAAGEWGLNNWK
LPNGALDMRHLTDAIHERGMTAVLWWRPCDGGREDSALFKEHPEYFIKNQDGSFGKLAGPGQWNSFLGSCGYALCPLSEGAVQ
SQVDFINRAMNEWGFDGFKSDYVWSLPKCYSQDHHHEYPEESTEQQAVFYRAVYEAMTDNDPNAFHLLCNCGTPQDYYSLPYV
TQVPTADPTSVDQTRRRVKAYKALCGDYFPVTTDHNEVWYPSTIGTGAILIEKRDLSGWEEEEYAKWLKIAQENQLHKGTFIG
DLYSYGYDPYETYTVYKDGIMYYAFYKDGNRYRPSGNPDIELKGLEDGKLYRIVDYVNNQVVATNVTSSNAVFSYPFSDYLLV
KAVEISEPDTDGPGPVPDPEGAVTVEENDPELVYTGDWVREENDGYHGGGARYTKEAEASVELAFYGTGAAWYGQHDVNFGSA
RIYIDGTYVKTVSCMGEPGINIKLFEISGLDLASHRIKIECETPVIDIDRLTYIKGEEVPAKVMTADLRALTVIANQYDMNSF
ADGNYKDQLGVSLVRANQLLAADDVTQGAVNEEQKYLLNAMLKIRKKVDKSWIGLPGPIPQDIQTENISRDNLAKVISYTGQL
DRDEIIPAIKEQLNDSYDKAVSIAERQDASQPEIDRAWAELMNAVQYSSYIRGSKEELLSLLDEYGKVDTTVYKDAALFIESL
EAAKKVYQDENAMDGEISDCIKQLRDAKDQLQLKDPVDPPKPDPDPDPKPDPTPDPGPDPKPDPTPDPTPDPKPNPTPTPDPT
PEPALKKPEQVSGLKSKAETDYLTVSWKKLNNAESYKVYIYKSGKWRLAGKTTKTSIKIKKLVSGTKYTVKVAAVNKAGQGKY
SSQVYTAAKPKKVKLKSVSRYRTSKVKLNYGKVKAGGYEIWMKNGKGSYKKAATSTKTTAIKSGLKKGKTYYFKVRAYVKNKN
QVIYGSFSNIKKYKMVL

### REFERENCES

Kuznetsova, I. M et al. Int J Mol Sci. (2014) “What Macromolecular Crowding Can Do to a Protein” 15(12): 23090-23140.
Marcus, D, M. et al. Biochem (1964) “Immunochemical Studies on Blood Groups. XXXI. Destruction of Blood Group A Activity by an Enzyme from Clostridium tertium Which Deacetylates N-Acetylgalactosamine in Intact Blood Group Substances” (4) 437-443.
Daniels, G. and Reid M. E. Transfusion (2010) “Blood groups: the past 50 years.” 50 (2):281-9. doi: 10.1111/j.1537−2995.2009.02456. x. Epub 2009 Nov. 9
Vox Sang. 2011 November; 101(4):327-32. doi: 10.1111/J.1423-0410.2011.01540.x. Epub 2011 Sep. 6.
Garratty, G. Vox Sang. (2008) “Modulating the red cell membrane to produce universal/stealth donor red cells suitable for transfusion.” 94(2):87-95. Epub 2007 Nov. 22.
Goldstein et al. Science (1982) “Group B erythrocytes enzymatically converted to group O survive normally in A, B, and O individuals.” 215(4529):168-70.
U.S. Pat. No. 4,609,627; and CA2272925
Kruskall M. S. et al. Transfusion (2000) “Transfusion to blood group A and O patients of group B RBCs that have been enzymatically converted to group O.” 40 (11):1290-8.
Clausen, H and Hakomori, S. Vox Sang. (1989) “ABH and related histo-blood group antigens; immunochemical differences in carrier isotypes and their distribution.” 56 (1):1-20.
EP2243793
Liu, Q. P. et al. J Biol Chem. (2008) “Identification of a GH110 subfamily of alpha 1,3-galactosidases: novel enzymes for removal of the alpha 3Gal xenotransplantation antigen.” 283(13):8545-54. doi: 10.1074/jbc.M709020200. Epub 2008 Jan. 28.
PCT/US1992/010113; and PCT/SE2015/050108
U.S. Pat. Nos. 4,088,538; 4,141,857; 4,206,259; 4,218,363; 4,229,536; 4,239,554; 4,619,897; 4,748,121; 4,749,653; 4,897,352; 4,954,444; 4,978,619; 5,154,808; 5,914,367; 5,962,279; 6,030,933; 6,291,582; 6,254,645; 10,016,490; and 10,041,055
Jeong, J. K. et al. J Bacteriol. (2009) “Characterization of the Streptococcus pneumoniae BgaC protein as a novel surface beta-galactosidase with specific hydrolysis activity for the Galbetai-3GlcNAc moiety of oligosaccharides.” 191(9):3011-23. doi: 10.1128/JB.01601-08. Epub 2009 Mar. 6.
Singh, A. K. et al. PLoS Pathog. (2014) “Unravelling the multiple functions of the architecturally intricate Streptococcus pneumoniae 3-galactosidase, BgaA.” 10(9): e1004364. doi: 10.1371/journal.ppat.1004364. eCollection 2014 September
Katayarna, T. et al. J Bacteriol. (2004) “Molecular cloning and characterization of Bifidobacterium bifidum 1,2-alpha-L-fucosidase (AfcA), a novel inverting glycosidase (glycoside hydrolase family 95).” 186(15):4885-93.
Williams, S. J. et al. J Biol Chem. (2002) “Aspartate 313 in the Streptomyces plicatus hexosaminidase plays a critical role in substrate-assisted catalysis by orienting the 2-acetamido group and stabilizing the transition state.” 277(42):40055-65. Epub 2002 Aug. 8.
Bolger, A. M. et al. Bioinformatics. (2014) “Trimmomatic: a flexible trimmer for Illumina sequence data.” 30 (15):2114-20. doi: 10.1093/bioinformatics/btu170. Epub 2014 Apr. 1.
Li 2013
Treangen, T. J. et al. Curr Protoc Bioinformatics (2011) “Next generation sequence assembly with AMOS.” Chapter 11:Unit 11.8. doi: 10.1002/0471250953bi1108s33
Hyatt, D. et al. BMC Bioinformatics. (2010) “Prodigal: prokaryotic gene recognition and translation initiation site identification.” 11:119. doi: 10.1186/1471-2105-11-119.
Konwar, K. M. et al. Bioinformatics. (2015) “MetaPathways v2.5: quantitative functional, taxonomic and usability improvements.” 31(20):3345-7. doi: 10.1093/bioinformatics/btv361. Epub 2015 Jun. 15.
Studier, F. W. Protein Expr Purif. (2005) “Protein production by auto-induction in high density shaking cultures.” 41 (1):207-34.
Palmier M. O. and Van Doren S. R. Anal Biochem. (2007) “Rapid determination of enzyme kinetics from fluorescence: overcoming the inner filter effect.” 371(1):43-51. Epub 2007 Jul. 18.
Kabsch, W. Acta Crystallogr D Biol Crystallogr. (2010) “XDS” 66(Pt 2):125-32. doi: 10.1107/S0907444909047337. Epub 2010 Jan. 22.
Evans, P. R. and Murshudov, G. N. Acta Crystallogr D Biol Crystallogr. (2013) “How good are my data and what is the resolution?” 69(Pt7):1204-14. doi: 10.1107/S0907444913000061. Epub 2013 Jun. 13.
Skubák, P. and Pannu, N. S. Nat Commun. (2013) “Automatic protein structure solution from weak X-ray data.” 4:2777. doi: 10.1038/ncomms3777.
Potterton, L. et al. Acta Crystallogr D Struct Biol. (2018) “CCP4i2: the new graphical user interface to the CCP4 program suite.” 74(Pt 2):68-84. doi: 10.1107/S2059798317016035. Epub 2018 Feb. 1.
Emsley, P. and Cowtan, K. Acta Crystallogr D Biol Crystallogr. (2004) “Coot: model-building tools for molecular graphics.” 60(Pt 12 Pt 1):2126-32. Epub 2004 Nov. 26.
Vagin, A. A. et al. Acta Crystallogr D Biol Crystallogr. (2004) “REFMAC5 dictionary: organization of prior chemical knowledge and guidelines for its use.” 60(Pt 12 Pt 1):2184-95. Epub 2004 Nov. 26.
Chen, V. B. et al. Acta Crystallogr D Biol Crystallogr. (2010) “MolProbity: all-atom structure validation for macromolecular crystallography.” 66(Pt 1):12-21. doi: 10.1107/S0907444909042073. Epub 2009 Dec. 21.
Zhang 2004
Vocadlo, D. J. et al. Biochemistry. (2002) “A case for reverse protonation: identification of Glu160 as an acid/base catalyst in Thermoanaerobacterium saccharolyticum beta-xylosidase and detailed kinetic analysis of a site-directed mutant.” 41 (31):9736-46.
Jones, D. R. et al. Biotechnol Biofuels. (2018) “SACCHARIS: an automated pipeline to streamline discovery of carbohydrate active enzyme activities within polyspecific families and de novo sequence datasets.” 11:27. doi: 10.1186/s13068-018-1027-x. eCollection 2018.
Yin, Y. et al. Nucleic Acids Res. (2012) “dbCAN: a web resource for automated carbohydrate-active enzyme annotation.” 40(Web Server issue):W445-51. doi: 10.1093/narigks479. Epub 2012 May 29.
Edgar, R. C. Bioinformatics. (2010) “Search and clustering orders of magnitude faster than BLAST.” 26(19):2460-1. doi: 10.1093/bioinformaticsibtq461. Epub 2010 Aug. 12.
Stamatakis, A. Bioinformatics. (2006) “RAxML-VI-HPC: maximum likelihood-based phylogenetic analyses with thousands of taxa and mixed models.” 22:2688-2690. doi:10.1093/bioinformatics/bt1446.
Stamatakis, A. and Ott, M. Philos Trans R Soc Lond B Biol Sci. (2008) “Efficient computation of the phylogenetic likelihood function on multi-gene alignments and multi-core architectures.” 363(1512):3977-84. doi: 10.1098/rstb.2008.0163.
Eddy, S. R. Bioinformatics. (1998) “Profile hidden Markov models.” 14(9):755-63. Review.
Capella-Gutiérrez, S. et al. Bioinformatics. (2009) “trimAl: a tool for automated alignment trimming in large-scale phylogenetic analyses.” 25(15):1972-3. doi: 10.1093/bioinformatics/btp348. Epub 2009 Jun. 8.
Matsen, F. A. et al. PLoS One. (2012) “A format for phylogenetic placements.” 7(2):e31009. doi: 10.1371/journal.pone.0031009. Epub 2012 Feb. 22.
Letunic, I. and Bork, P. Nucleic Acids Res. (2016) “Interactive tree of life (iTOL) v3: an online tool for the display and annotation of phylogenetic and other trees.” 44(W1):W242-5. doi: 10.1093/nar/gkw290. Epub 2016 Apr. 19.
Engler, C. et al. PLoS One. (2008) “A one pot, one step, precision cloning method with high throughput capability.” 3(11):e3647. doi: 10.1371/journal.pone.0003647. Epub 2008 Nov. 5.
Kwan, D. H. et al. J Am Chem Soc. (2015) “Toward Efficient Enzymes for the Generation of Universal Blood through Structure-Guided Directed Evolution.” 137(17):5695-705. doi: 10.10201/ja5116088. Epub 2015 Apr. 24. The eleven fosmids were sequenced on an Illumina MiSeg™ and ORFs therein that are present in the CAZy™ database (http://www.cazy.org/)(Lombard 2014
Konwar, K. M. et al. Bioinformatics. (2015) “MetaPathways v2.5: quantitative functional, taxonomic and usability improvements.” 31(20):3345-7. doi: 10.1093/bioinformatics/btv361. Epub 2015 Jun. 15.
Li, D. et al. Bioinformatics. (2015) “MEGAHIT: an ultra-fast single-node solution for large and complex metagenomics assembly via succinct de Bruijn graph.” 31 (10):1674-6. doi: 10.1093/bioinformatics/btv033. Epub 2015 Jan. 20.
enzymes from GH4 Add Yip, V. L. and Withers, S. G. J. Amer. Chem. Soc. (2006) “Mechanistic analysis of the unusual redox-elimination sequence employed by Thermotoga maritima BglT: a 6-phospho-beta-glucosidase from glycoside hydrolase family 4.” 126, 8354⁻⁸³⁵⁵
Chapanian, R. et al. Nat Commun. (2014) “Enhancement of biological reactions on cell surfaces via macromolecular crowding.” 5:4683. doi: 10.1038/ncomms5683.
Varrot, A. et al. J Mol Biol. (2005) “NAD+ and metal-ion dependent hydrolysis by family 4 glycosidases: structural insight into specificity for phospho-beta-D-glucosides.” 346 (2):423-35. Epub 2005 Jan. 7.
Liu, Q. P. et al. Nat Biotechnol. (2007) “Bacterial glycosidases for the production of universal red blood cells.” 25(4):454-64. Epub 2007 Apr. 1.
Comfort, D. A. et al. Biochemistry (2007) “Biochemical analysis of Thermotoga maritima GH36 alpha-galactosidase (TmGalA) confirms the mechanistic commonality of clan GH-D glycoside hydrolases.” 46(11):3319-30. Epub 2007 Feb. 27.
Calcutt, M. J. et al. FEMS Microbiol Lett. (2002) “Identification, molecular cloning and expression of an alpha-N-acetylgalactosaminidase gene from Clostridium perfringens.” 214(1):77-80.
Gerbal, A. Maslet, C. and Salmon, C. Vox Sang. (1975) “Immunological aspects of the acquired B antigen.” 28(5):398-403.
Judd, W. J. and Annesley, T. M Transfusion medicine reviews (1996) “The acquired-B phenomenon.” 10, 111-117.
Kelley, L. A. et al. Nat Protoc. (2015) “The Phyre2 web portal for protein modeling, prediction and analysis.” 10(6):845-58. doi: 10.1038/nprot.2015.053. Epub 2015 May 7.
Ficko-Blean, E. and Boraston, AB. J Biol Chem. (2006) “The interaction of a carbohydrate-binding module from a Clostridium perfringens N-acetyl-beta-hexosaminidase with its carbohydrate receptor.” 281(49):37748-57. Epub 2006 Sep. 21.
Cohen, M. et al. Blood. (2009) “ABO blood group glycans modulate sialic acid recognition on erythrocytes.” 114(17):3668-76. doi: 10.1182/blood-2009-06-227041. Epub 2009 Aug. 24.
Fredslund, F. et al. J Mol Biol. (2011) “Crystal structure of α-galactosidase from Lactobacillus acidophilus NCFM: insight into tetramer formation and substrate binding.” 412(3):466-80. doi: 10.1016/j.jmb.2011.07.057. Epub 2011 Jul. 30.
Guibert, E. E. et al. Transfus Med Hemother. (2011) “Organ Preservation: Current Concepts and New Strategies for the Next Decade” 38 (2): 125-142.

What is claimed is:

1. A perfusion fluid for enzymatically cleaving A-antigens from a donor organ comprising:
(a) a purified GalNAcDeacetylase protein; and
(b) a purified Galactosaminidase protein.

2. The perfusion fluid of claim 1, wherein:
(a) the GalNAcDeacetylase is a purified protein selected from one or more of: SEQ ID NO.:2; SEQ ID NO.:4; SEQ ID NO.:5; SEQ ID NO.:17; SEQ ID NO.:23; SEQ ID NO.:29; SEQ ID NO.:31; SEQ ID NO.:32; SEQ ID NO.:33; SEQ ID NO.:34; and SEQ ID NO.:35; and
(b) the Galactosaminidase is a purified protein is selected from one or more of the following: SEQ ID NO.:7; SEQ ID NO.:9; SEQ ID NO.:10; SEQ ID NO.:19; SEQ ID NO.:21; SEQ ID NO.:36; and SEQ ID NO.:37.

3. The perfusion fluid of claim 1, wherein the perfusion fluid comprises: a purified enzyme having a GalNAcDeacetylase activity consisting essentially of an amino acid sequence at least 90% identical to the sequence set forth in one of SEQ ID NOs:2, 4, 5, 17, 23, 29, 31 and 32-35; and a purified enzyme having Galactosaminidase activity consisting essentially of an amino acid sequence at least 90% identical to the sequence set forth in one of SEQ ID NOs:7, 9, 10, 19, 21, 36 and 37.

4. The perfusion fluid of claim 1 or 2, wherein the perfusion fluid comprises enzymes selected from one or more of:
(a) the purified GalNAcDeacetylase protein is a purified Flavonifractor plautii GalNAcDeacetylase protein of SEQ ID NO.:2, SEQ ID NO.:4 and SEQ ID NO.:5; and
(b) the purified Galactosaminidase protein is a purified Flavonifractor plautii Galactosaminidase protein of SEQ ID NO.:7, SEQ ID NO.:9 and SEQ ID NO.:10.

5. The perfusion fluid of claim 1 or 2, wherein the perfusion fluid comprises one or more of:
(a) the purified GalNAcDeacetylase protein is a purified Clostridium tertium GalNAcDeacetylase protein of SEQ ID NO.:17 or SEQ ID NO.:32; and
(b) the purified Galactosaminidase protein is a purified Clostridium tertium Galactosaminidase protein of SEQ ID NO.:19 or SEQ ID NO.:36.

6. The perfusion fluid of any one of claims 1-5, wherein the GalNAcDeacetylase and Galactosaminidase are capable of cleaving A-antigen at or below 1 μg/ml.

7. The perfusion fluid of any one of claims 1-6, wherein the GalNAcDeacetylase and Galactosaminidase have A-antigen cleaving activity at a pH between about 6.5 and about 7.5.

8. The perfusion fluid of any one of claims 1-7, wherein the GalNAcDeacetylase and Galactosaminidase have A-antigen cleaving activity at a temperatures between 4° C. and 37° C.

9. The perfusion fluid of any one of claims 1-8, wherein the perfusion fluid further comprises a buffered extracellular solution.

10. The perfusion fluid of claim 9, wherein the buffered extracellular solution is selected from: Steen™; Perfadex™; Perfadex Plus™; EuroCollins solution; Histidine-Tryptophan-Ketoglutarate (HTK) solution; University of Wisconsin solution (UW); Celsior solution; Kidney Perfusion solution (KPS-1); Kyoto University solution; IGL-1 solution; and Citrate solution.

11. A method for enzymatically cleaving A-antigens ex vivo from a donor organ, the method comprising:
(a) perfusing a donor organ displaying type A antigen with a fluid comprising GalNAcDeacetylase protein and a Galactosaminidase protein for a period of time sufficient to allow the enzymes to cleave A-antigens from the donor organ; or
(b) incubating a donor organ displaying type A antigen with a fluid comprising GalNAcDeacetylase protein and a Galactosaminidase protein for a period of time sufficient to allow the enzymes to cleave A-antigens from the donor organ.

12. The method of claim 11, wherein the GalNAcDeacetylase is a purified protein selected from one or more of: SEQ ID NO.:2; SEQ ID NO.:4; SEQ ID NO.:5; SEQ ID NO.:17; SEQ ID NO.:23; SEQ ID NO.:29; SEQ ID NO.:31; SEQ ID NO.:32; SEQ ID NO.:33; SEQ ID NO.:34; and SEQ ID NO.:35; and the Galactosaminidase is a purified protein is selected from one or more of the following: SEQ ID NO.:7; SEQ ID NO.:9; SEQ ID NO.:10; SEQ ID NO.:19; SEQ ID NO.:21; SEQ ID NO.:36; and SEQ ID NO.:37.

13. The method of claim 11, wherein the composition comprises: a purified enzyme having GalNAcDeacetylase activity consisting essentially of an amino acid sequence at least 90% identical to the sequence set forth in one of SEQ ID NOs:2, 4, 5, 17, 23, 29, 31 and 32-35; and a purified enzyme having Galactosaminidase activity consisting essentially of an amino acid sequence at least 90% identical to the sequence set forth in one of SEQ ID NOs: 7, 9, 10, 19, 21, 36 and 37.

14. The method of claim 11, wherein the GalNAcDeacetylase is a purified Flavonifractor plautii GalNAcDeacetylase protein of SEQ ID NO.:4 or SEQ ID NO.:5 and the Galactosaminidase is a purified Flavonifractor plautii Galactosaminidase protein of SEQ ID NO.:9 or SEQ ID NO.:10.

15. The method of any one of claims 11-14, wherein the GalNAcDeacetylase protein and the Galactosaminidase protein are in a buffered extracellular solution.

16. The method of claim 15, wherein the buffered extracellular solution is selected from: Steen™; Perfadex™; Perfadex Plus™; EuroCollins solution; Histidine-Tryptophan-Ketoglutarate (HTK) solution; University of Wisconsin solution (UW); Celsior solution; Kidney Perfusion solution (KPS-1); Kyoto University solution; IGL-1 solution; and Citrate solution.

17. The method of any one of claims 11-16, wherein the donor organ is a solid organ.

18. The method of claim 17, wherein the solid organ is selected from one of the following: lung; kidney; liver; heart; pancreas; and intestine.

19. The method of claim 18, wherein the solid organ is a lung.

20. The method of claim 17, wherein the GalNAcDeacetylase protein and the Galactosaminidase protein is mixed with an ex vivo buffered extracellular lung solution and circulated through the lung, whereby the GalNAcDeacetylase protein and the Galactosaminidase protein are in contact with the vasculature of the donor organ for a period of time sufficient to substantially clear the A-antigens from the vasculature of the lung.

21. The method of claim 20, wherein the time to clear the A-antigens from the vasculature of the lung is about 1 hour.

22. The method of any one of claims 11-21, wherein the method further comprises washing the donor organ to remove GalNAcDeacetylase, Galactosaminidase and cleaved A-antigens.

23. The method of any one of claims 11-22, wherein the GalNAcDeacetylase and Galactosaminidase are capable of cleaving A-antigen at or below 1 μg/ml.

24. The method of any one of claims 11-23, wherein the GalNAcDeacetylase and Galactosaminidase have A-antigen cleaving activity at a pH between about 6.5 and about 7.5.

25. The method of any one of claims 11-24, wherein the GalNAcDeacetylase and Galactosaminidase have A-antigen cleaving activity at a temperatures between 4° C. and 37° C.


SEQUENCE LISTING (108 sequences)

SEQ ID NO: 1 - DNA, 2319 bp, Flavonifractor plautii
atgagaaatcgaaggaaagctgtttcgcttctaacgggcctactcgtgacggcccagttatttccaaccgcggcgcttgcggcagactccagcgagtccgcattgaacaaggcccccggatatcaggattttcccgcctattacagcgacagtgcgcatgccgatgaccaggtgactcacccggacgtagttgtcctggaagaaccgtggaacggctatcgctattgggccgtttatacgcccaacgtgatgcggatctccatctacgaaaacccgtccatcgttgcctccagcgacggagtgcattgggtagaaccggaggggctttccaatcccattgagccgcagccgcccagcacccgctaccacaactgcgacgctgatatggtctataacgcggaatacgatgccatgatggcctattggaactgggcggatgaccagggcggaggcgttggggccgaagtccggctgcggatttcctatgacggcgtacattggggcgtccccgtgacttatgatgagatgacccgcgtatggtcgaagcccacctccgacgcggagcgtcaggttgcggatggagaggatgacttcattaccgccattgcttctccagaccgctacgatatgctctctcccactattgtctacgatgacttccgggatgtgttcatcctgtgggccaacaataccggcgacgtggggtatcagaatggtcaggcgaacttcgtggaaatgcgttattcggacgacgggatcacctggggtgagccagtccgcgtcaacggcttcctggggcttgacgagaatgggcagcagttggccccctggcatcaggatgtccagtatgttccagatttgaaggagtttgtttgtatttcccagtgctttgccggccgaaatccggatggctctgtcctgcacctgaccacatcaaaggatggagtcaactgggagcaggtgggcaccaagcccctgctgtcccccgggccagacggcagttgggatgatttccagatctatcgctccagtttttactatgagccaggcagttccgccggagatggtaccatgcgcgtctggtacagtgccctgcagaaggacaccaataacaagatggtcgcggattcctccgggaatctgaccattcaggccaaaagtgaggatgaccgcatctggaggatcggctatgcggaaaacagttttgttgagatgatgcgcgtgctgctggatgaccccggctacacgacgcccgccctggtttccggcaattcccttatgctgagtgctgagaccacttcccttcccacaggggatgtcatgaagctggaaaccagtttcgcgcctgtggacacctctgatcaggtcgtgaaatatacctccagtgatccggatgtggcgacggtggatgagtttggaaccattacaggcgtttctgtcggttcagcgcgcatcatggcggagacccgggagggcctgtccgacgaccttgaaattgcagtggtggagaatccgtacacgctgattccccagtccaatatgacggcaaccgccaccagcgtctacggcgggacgacggagggccccgcctccaatgtcctcgatggaaacgtccgcacaatatggcataccaactatgctcccaaagatgaactgccgcagagtatcaccgtttcctttgaccagccctataccgtcggccgcttcgtctataccccacgtcaaaacgggacaaatggcataatttcggagtatgagctatacgccatccaccaggacggcagcaaggacctagtcgcctccggctcagactgggcgctcgatgccaaggataaaaccgtgagctttgcaccggtagaagccgtcggcctggagctcaaggcgattgccggcgcaggtgggttcggtactgccgccgaactcaatgtgtatgcgtatggtccaatcgagcctgcgcccgtatatgtcccggtggatgaccgggatgcttccctggtgtttacgggtgcatggaatagcgacagcaacggaagcttttatgaagggacggcccgttataccaacgagatcggcgcgtccgtggagttcacatttgtggggacggccattcggtggtatggtcaaaatgatgtaaatttcggcgctgcggaggtatacgtggacggcgttctggcaggggaggtaaatgtgtatgggccggcggcggctcagcagcttctatttgaggcggacggtctggcctatgggaagcataccatccgcatcgtctgtgtgtctccggtggttgacttcgactatttttcgtatgtgggagaataa

SEQ ID NO: 2 - protein, 772 aa, Flavonifractor plautii
MRNRRKAVSLLTGLLVTAQLFPTAALAADSSESALNKAPGYQDFPAYYSDSAHADDQVTHPDVVVLEEPWNGYRYWAVYTPNVMRISIYENPSIVASSDGVHWVEPEGLSNPIEPQPPSTRYHNCDADMVYNAEYDAMMAYWNWADDQGGGVGAEVRLRISYDGVHWGVPVTYDEMTRVWSKPTSDAERQVADGEDDFITAIASPDRYDMLSPTIVYDDFRDVFILWANNTGDVGYQNGQANFVEMRYSDDGITWGEPVRVNGFLGLDENGQQLAPWHQDVQYVPDLKEFVCISQCFAGRNPDGSVLHLTTSKDGVNWEQVGTKPLLSPGPDGSWDDFQIYRSSFYYEPGSSAGDGTMRVWYSALQKDTNNKMVADSSGNLTIQAKSEDDRIWRIGYAENSFVEMMRVLLDDPGYTTPALVSGNSLMLSAETTSLPTGDVMKLETSFAPVDTSDQVVKYTSSDPDVATVDEFGTITGVSVGSARIMAETREGLSDDLEIAVVENPYTLIPQSNMTATATSVYGGTTEGPASNVLDGNVRTIWHTNYAPKDELPQSITVSFDQPYTVGRFVYTPRQNGTNGIISEYELYAIHQDGSKDLVASGSDWALDAKDKTVSFAPVEAVGLELKAIAGAGGFGTAAELNVYAYGPIEPAPVYVPVDDRDASLVFTGAWNSDSNGSFYEGTARYTNEIGASVEFTFVGTAIRWYGQNDVNFGAAEVYVDGVLAGEVNVYGPAAAQQLLFEADGLAYGKHTIRIVCVSPVVDFDYFSYVGE

SEQ ID NO: 3 - DNA, 2238 bp, Flavonifractor plautii
gcagactccagcgagtccgcattgaacaaggcccccggatatcaggattttcccgcctattacagcgacagtgcgcatgccgatgaccaggtgactcacccggacgtagttgtcctggaagaaccgtggaacggctatcgctattgggccgtttatacgcccaacgtgatgcggatctccatctacgaaaacccgtccatcgttgcctccagcgacggagtgcattgggtagaaccggaggggctttccaatcccattgagccgcagccgcccagcacccgctaccacaactgcgacgctgatatggtctataacgcggaatacgatgccatgatggcctattggaactgggcggatgaccagggcggaggcgttggggccgaagtccggctgcggatttcctatgacggcgtacattggggcgtccccgtgacttatgatgagatgacccgcgtatggtcgaagcccacctccgacgcggagcgtcaggttgcggatggagaggatgacttcattaccgccattgcttctccagaccgctacgatatgctctctcccactattgtctacgatgacttccgggatgtgttcatcctgtgggccaacaataccggcgacgtggggtatcagaatggtcaggcgaacttcgtggaaatgcgttattcggacgacgggatcacctggggtgagccagtccgcgtcaacggcttcctggggcttgacgagaatgggcagcagttggccccctggcatcaggatgtccagtatgttccagatttgaaggagtttgtttgtatttcccagtgctttgccggccgaaatccggatggctctgtcctgcacctgaccacatcaaaggatggagtcaactgggagcaggtgggcaccaagcccctgctgtcccccgggccagacggcagttgggatgatttccagatctatcgctccagtttttactatgagccaggcagttccgccggagatggtaccatgcgcgtctggtacagtgccctgcagaaggacaccaataacaagatggtcgcggattcctccgggaatctgaccattcaggccaaaagtgaggatgaccgcatctggaggatcggctatgcggaaaacagttttgttgagatgatgcgcgtgctgctggatgaccccggctacacgacgcccgccctggtttccggcaattcccttatgctgagtgctgagaccacttcccttcccacaggggatgtcatgaagctggaaaccagtttcgcgcctgtggacacctctgatcaggtcgtgaaatatacctccagtgatccggatgtggcgacggtggatgagtttggaaccattacaggcgtttctgtcggttcagcgcgcatcatggcggagacccgggagggcctgtccgacgaccttgaaattgcagtggtggagaatccgtacacgctgattccccagtccaatatgacggcaaccgccaccagcgtctacggcgggacgacggagggccccgcctccaatgtcctcgatggaaacgtccgcacaatatggcataccaactatgctcccaaagatgaactgccgcagagtatcaccgtttcctttgaccagccctataccgtcggccgcttcgtctataccccacgtcaaaacgggacaaatggcataatttcggagtatgagctatacgccatccaccaggacggcagcaaggacctagtcgcctccggctcagactgggcgctcgatgccaaggataaaaccgtgagctttgcaccggtagaagccgtcggcctggagctcaaggcgattgccggcgcaggtgggttcggtactgccgccgaactcaatgtgtatgcgtatggtccaatcgagcctgcgcccgtatatgtcccggtggatgaccgggatgcttccctggtgtttacgggtgcatggaatagcgacagcaacggaagcttttatgaagggacggcccgttataccaacgagatcggcgcgtccgtggagttcacatttgtggggacggccattcggtggtatggtcaaaatgatgtaaatttcggcgctgcggaggtatacgtggacggcgttctggcaggggaggtaaatgtgtatgggccggcggcggctcagcagcttctatttgaggcggacggtctggcctatgggaagcataccatccgcatcgtctgtgtgtctccggtggttgacttcgactatttttcgtatgtgggagaataa

SEQ ID NO: 4 - protein, 745 aa, Flavonifractor plautii
ADSSESALNKAPGYQDFPAYYSDSAHADDQVTHPDVVVLEEPWNGYRYWAVYTPNVMRISIYENPSIVASSDGVHWVEPEGLSNPIEPQPPSTRYHNCDADMVYNAEYDAMMAYWNWADDQGGGVGAEVRLRISYDGVHWGVPVTYDEMTRVWSKPTSDAERQVADGEDDFITAIASPDRYDMLSPTIVYDDFRDVFILWANNTGDVGYQNGQANFVEMRYSDDGITWGEPVRVNGFLGLDENGQQLAPWHQDVQYVPDLKEFVCISQCFAGRNPDGSVLHLTTSKDGVNWEQVGTKPLLSPGPDGSWDDFQIYRSSFYYEPGSSAGDGTMRVWYSALQKDTNNKMVADSSGNLTIQAKSEDDRIWRIGYAENSFVEMMRVLLDDPGYTTPALVSGNSLMLSAETTSLPTGDVMKLETSFAPVDTSDQVVKYTSSDPDVATVDEFGTITGVSVGSARIMAETREGLSDDLEIAVVENPYTLIPQSNMTATATSVYGGTTEGPASNVLDGNVRTIWHTNYAPKDELPQSITVSFDQPYTVGRFVYTPRQNGTNGIISEYELYAIHQDGSKDLVASGSDWALDAKDKTVSFAPVEAVGLELKAIAGAGGFGTAAELNVYAYGPIEPAPVYVPVDDRDASLVFTGAWNSDSNGSFYEGTARYTNEIGASVEFTFVGTAIRWYGQNDVNFGAAEVYVDGVLAGEVNVYGPAAAQQLLFEADGLAYGKHTIRIVCVSPVVDFDYFSYVGE

SEQ ID NO: 5 - protein, 760 aa, Flavonifractor plautii
MGHHHHHHHHHHSSGADSSESALNKAPGYQDFPAYYSDSAHADDQVTHPDVVVLEEPWNGYRYWAVYTPNVMRISIYENPSIVASSDGVHWVEPEGLSNPIEPQPPSTRYHNCDADMVYNAEYDAMMAYWNWADDQGGGVGAEVRLRISYDGVHWGVPVTYDEMTRVWSKPTSDAERQVADGEDDFITAIASPDRYDMLSPTIVYDDFRDVFILWANNTGDVGYQNGQANFVEMRYSDDGITWGEPVRVNGFLGLDENGQQLAPWHQDVQYVPDLKEFVCISQCFAGRNPDGSVLHLTTSKDGVNWEQVGTKPLLSPGPDGSWDDFQIYRSSFYYEPGSSAGDGTMRVWYSALQKDTNNKMVADSSGNLTIQAKSEDDRIWRIGYAENSFVEMMRVLLDDPGYTTPALVSGNSLMLSAETTSLPTGDVMKLETSFAPVDTSDQVVKYTSSDPDVATVDEFGTITGVSVGSARIMAETREGLSDDLEIAVVENPYTLIPQSNMTATATSVYGGTTEGPASNVLDGNVRTIWHTNYAPKDELPQSITVSFDQPYTVGRFVYTPRQNGTNGIISEYELYAIHQDGSKDLVASGSDWALDAKDKTVSFAPVEAVGLELKAIAGAGGFGTAAELNVYAYGPIEPAPVYVPVDDRDASLVFTGAWNSDSNGSFYEGTARYTNEIGASVEFTFVGTAIRWYGQNDVNFGAAEVYVDGVLAGEVNVYGPAAAQQLLFEADGLAYGKHTIRIVCVSPVVDFDYFSYVGE

SEQ ID NO: 6 - DNA, 3159 bp, Flavonifractor plautii
gcggcgcctgcaacggacaccggcaacgcaggactgattgcagaaggtgattatgccattgccggcaatggcgtccgcgtcacttatgacgcggacgggcagacaatcactctgtaccgcacagagggatctgggcttatccagatgagcaagccttctccattgggagggccagtgattggagggcaggaggttcaggacttcagccatatttcatgtgatgtggagcagagcaccagcggagtgatgggcagcggtcagagaatgaccattacctctcagagcatgagcacgggcctaattcgtacctatgtgctggagacctctgatatcgaggagggtgtggtatatactgcaacatcctatgaggcaggagcttctgatgtggaagtgtcttggttcattggcagtgtgtatgagctttatggtgcggaagatcgtatctggagttataacggcggcggtgaggggccgatgcactactatgatacgcttcaaaagattgacctgaccgactctggcaagttcagtagggagaataaacaggatgacacggctgcaagtattcctgtgtcagatatttacattgctgatggagggattaccgttggcgatgcttctgcaaccagaagggaggtacatactccggttcaggaaaccagtgattcagctcaagtttctatcgggtggccaggcaaagtcattgccgccggaagcgtgatcgaaattggtgagagctttgctgtagtccatccgggtgactattataacggcttgagaggttacaaaaatgcaatggatcacttgggcgtgattatgcctgcacctggggatattcctgatagcagctatgatctccgatgggaaagctggggctgggggtttaactggacgatcgatttaataatcggcaaattggatgaacttcaggcagccggagtcaagcagatcactttggatgatggttggtataccaatgcaggagactgggccttaaatccagaaaagtttccaaatggagcctccgatgcgttgcggctgacagatgcaattcatgagcatggtatgactgcactcctttggtggagaccttgtgacggcgggatcgatagtatactctatcagcaacaccctgaatatttcgttatggatgcagatggaagacctgcaaggcttcctactcctggtggtgggaccaatcccagcttgggatatgcactttgccctatggcggatggtgcgattgcaagccaagttgactttgtaaaccgtgcaatgaatgattgggggttcgatggcttcaagggagattatgtgtggagtatgcctgaatgctacaatcctgcacataaccacgcctcgccagaagaatccactgaaaagcaatccgagatataccgcgtctcttatgaggctatggtggccaacgaccccaatgtgttcaatttgttgtgcaactgcggtacgccccaggactactatagtttaccatatatgacacagattgctacggctgaccccacttctgtggatcaaacaaggagacgcgtgaaagcctacaaggcactgatgggagattatttccctgttacagccgaccacaataacatctggtatccaagtgccgtcggtacgggctctgttctcattgaaaaacgtgaccttagcggtactgccaaggaagaatatgaaaaatggcttgggattgcggatacagttcagttgcagaaaggccggtttattggcgatctttacagttatggttttgacccttacgaaacctatgtggtggagaaagacggggttatgtactatgccttctacaaagatgggagcaaatatagccccactggctatccagatattgagttgaaggggctagatccaaataaaatgtataggattgttgactatgtcaatgatcgtgtcgtggcaacaaacctgatgggtgataacgctgtattcaatacacgtttttccgactatctactggttaaagcggtggaaatttcggaaccggatccagaacctgttgaccctgattatggtttcacctctgttgatgacagagacgaggctcttatttacacagggacatggcatgatgacaataacgcatctttcagcgaagggactgcacgttataccaacagtacggatgcttcggttgtattctcctttactggaacttccattcgctggtatggccagagggataccaattttggcacggcagaagtttatttggacgatgaactgaaaacaacagttgatgcgaatggggccgcagaagcaggcgtatgtctttttgaggcgcttgatcttccggctgccgagcataccattaaaattgtgtgcaagagcggagtgattgatattgaccgctttgcatatgaagctgctacccttgaacccatctatgaaaaggtcgatgcgctctcggatcggatcacttatgttgggaattgggaagagtatcacaacagcgagttctacatgggaaacgcaatgcgcacagacgaagccggcgcttatgctgaactgactttccgtggtacagccgtacgcctgtatgcagagatgagcttcaattttggcactgcagatgtctatttagacggagagttagtggaaaacataatcctatacggccaggaagcaactgggcagctaatgtttgagcgtacgggactggaggaaggagaacataccattcgccttgtacaaaacgcctggaacatcaatttggactatatttcttatctaccagagcaagatcaaccaacgccgccggagacgacggttactgttgatgcaatggacgcccaactggtgtatacaggcgtatggaatgatgactatcatgacgtctttcaggaaggaaccgcccgttatgccagtagtgccggcgcctcggtcgagttcgaatttactggaagcgaaatccgttggtatggacaaaatgattccaacttcggtgttgccagcgtttatatcgataatgagtttgtgcagcaggtaaatgttaacggagctgcggctgtgggaaagcttttgtttcaaaaggctgatctaccagccggttcgcacacgatccgcattgtgtgcgatactccggttattgatttggactatttgacttataccactaacgcataa

SEQ ID NO: 7 - protein, 1078 aa, Flavonifractor plautii
MRGKKFISLTLSTMLCLQLLPTASFAAAPATDTGNAGLIAEGDYAIAGNGVRVTYDADGQTITLYRTEGSGLIQMSKPSPLGGPVIGGQEVQDFSHISCDVEQSTSGVMGSGQRMTITSQSMSTGLIRTYVLETSDIEEGVVYTATSYEAGASDVEVSWFIGSVYELYGAEDRIWSYNGGGEGPMHYYDTLQKIDLTDSGKFSRENKQDDTAASIPVSDIYIADGGITVGDASATRREVHTPVQETSDSAQVSIGWPGKVIAAGSVIEIGESFAVVHPGDYYNGLRGYKNAMDHLGVIMPAPGDIPDSSYDLRWESWGWGFNWTIDLIIGKLDELQAAGVKQITLDDGWYTNAGDWALNPEKFPNGASDALRLTDAIHEHGMTALLWWRPCDGGIDSILYQQHPEYFVMDADGRPARLPTPGGGTNPSLGYALCPMADGAIASQVDFVNRAMNDWGFDGFKGDYVWSMPECYNPAHNHASPEESTEKQSEIYRVSYEAMVANDPNVFNLLCNCGTPQDYYSLPYMTQIATADPTSVDQTRRRVKAYKALMGDYFPVTADHNNIWYPSAVGTGSVLIEKRDLSGTAKEEYEKWLGIADTVQLQKGRFIGDLYSYGFDPYETYVVEKDGVMYYAFYKDGSKYSPTGYPDIELKGLDPNKMYRIVDYVNDRVVATNLMGDNAVFNTRFSDYLLVKAVEISEPDPEPVDPDYGFTSVDDRDEALIYTGTWHDDNNASFSEGTARYTNSTDASVVFSFTGTSIRWYGQRDTNFGTAEVYLDDELKTTVDANGAAEAGVCLFEALDLPAAEHTIKIVCKSGVIDIDRFAYEAATLEPIYEKVDALSDRITYVGNWEEYHNSEFYMGNAMRTDEAGAYAELTFRGTAVRLYAEMSFNFGTADVYLDGELVENIILYGQEATGQLMFERTGLEEGEHTIRLVQNAWNINLDYISYLPEQDQPTPPETTVTVDAMDAQLVYTGVWNDDYHDVFQEGTARYASSAGASVEFEFTGSEIRWYGQNDSNFGVASVYIDNEFVQQVNVNGAAAVGKLLFQKADLPAGSHTIRIVCDTPVIDLDYLTYTTNA

SEQ ID NO: 8 - DNA, 3159 bp, Flavonifractor plautii; identical to SEQ ID NO: 6

SEQ ID NO: 9 - protein, 1052 aa, Flavonifractor plautii
AAPATDTGNAGLIAEGDYAIAGNGVRVTYDADGQTITLYRTEGSGLIQMSKPSPLGGPVIGGQEVQDFSHISCDVEQSTSGVMGSGQRMTITSQSMSTGLIRTYVLETSDIEEGVVYTATSYEAGASDVEVSWFIGSVYELYGAEDRIWSYNGGGEGPMHYYDTLQKIDLTDSGKFSRENKQDDTAASIPVSDIYIADGGITVGDASATRREVHTPVQETSDSAQVSIGWPGKVIAAGSVIEIGESFAVVHPGDYYNGLRGYKNAMDHLGVIMPAPGDIPDSSYDLRWESWGWGFNWTIDLIIGKLDELQAAGVKQITLDDGWYTNAGDWALNPEKFPNGASDALRLTDAIHEHGMTALLWWRPCDGGIDSILYQQHPEYFVMDADGRPARLPTPGGGTNPSLGYALCPMADGAIASQVDFVNRAMNDWGFDGFKGDYVWSMPECYNPAHNHASPEESTEKQSEIYRVSYEAMVANDPNVFNLLCNCGTPQDYYSLPYMTQIATADPTSVDQTRRRVKAYKALMGDYFPVTADHNNIWYPSAVGTGSVLIEKRDLSGTAKEEYEKWLGIADTVQLQKGRFIGDLYSYGFDPYETYVVEKDGVMYYAFYKDGSKYSPTGYPDIELKGLDPNKMYRIVDYVNDRVVATNLMGDNAVFNTRFSDYLLVKAVEISEPDPEPVDPDYGFTSVDDRDEALIYTGTWHDDNNASFSEGTARYTNSTDASVVFSFTGTSIRWYGQRDTNFGTAEVYLDDELKTTVDANGAAEAGVCLFEALDLPAAEHTIKIVCKSGVIDIDRFAYEAATLEPIYEKVDALSDRITYVGNWEEYHNSEFYMGNAMRTDEAGAYAELTFRGTAVRLYAEMSFNFGTADVYLDGELVENIILYGQEATGQLMFERTGLEEGEHTIRLVQNAWNINLDYISYLPEQDQPTPPETTVTVDAMDAQLVYTGVWNDDYHDVFQEGTARYASSAGASVEFEFTGSEIRWYGQNDSNFGVASVYIDNEFVQQVNVNGAAAVGKLLFQKADLPAGSHTIRIVCDTPVIDLDYLTYTTNA

SEQ ID NO: 10 - protein, 1067 aa, Flavonifractor plautii
MGHHHHHHHHHHSSGAAPATDTGNAGLIAEGDYAIAGNGVRVTYDADGQTITLYRTEGSGLIQMSKPSPLGGPVIGGQEVQDFSHISCDVEQSTSGVMGSGQRMTITSQSMSTGLIRTYVLETSDIEEGVVYTATSYEAGASDVEVSWFIGSVYELYGAEDRIWSYNGGGEGPMHYYDTLQKIDLTDSGKFSRENKQDDTAASIPVSDIYIADGGITVGDASATRREVHTPVQETSDSAQVSIGWPGKVIAAGSVIEIGESFAVVHPGDYYNGLRGYKNAMDHLGVIMPAPGDIPDSSYDLRWESWGWGFNWTIDLIIGKLDELQAAGVKQITLDDGWYTNAGDWALNPEKFPNGASDALRLTDAIHEHGMTALLWWRPCDGGIDSILYQQHPEYFVMDADGRPARLPTPGGGTNPSLGYALCPMADGAIASQVDFVNRAMNDWGFDGFKGDYVWSMPECYNPAHNHASPEESTEKQSEIYRVSYEAMVANDPNVFNLLCNCGTPQDYYSLPYMTQIATADPTSVDQTRRRVKAYKALMGDYFPVTADHNNIWYPSAVGTGSVLIEKRDLSGTAKEEYEKWLGIADTVQLQKGRFIGDLYSYGFDPYETYVVEKDGVMYYAFYKDGSKYSPTGYPDIELKGLDPNKMYRIVDYVNDRVVATNLMGDNAVFNTRFSDYLLVKAVEISEPDPEPVDPDYGFTSVDDRDEALIYTGTWHDDNNASFSEGTARYTNSTDASVVFSFTGTSIRWYGQRDTNFGTAEVYLDDELKTTVDANGAAEAGVCLFEALDLPAAEHTIKIVCKSGVIDIDRFAYEAATLEPIYEKVDALSDRITYVGNWEEYHNSEFYMGNAMRTDEAGAYAELTFRGTAVRLYAEMSFNFGTADVYLDGELVENIILYGQEATGQLMFERTGLEEGEHTIRLVQNAWNINLDYISYLPEQDQPTPPETTVTVDAMDAQLVYTGVWNDDYHDVFQEGTARYASSAGASVEFEFTGSEIRWYGQNDSNFGVASVYIDNEFVQQVNVNGAAAVGKLLFQKADLPAGSHTIRIVCDTPVIDLDYLTYTTNA

SEQ ID NO: 11 - DNA, 3963 bp, Clostridium tertium
atgaaaaaaagaattttagctacttttattacagctatgtgtggactgggatttttttcaaactggacttcaagtaatgcttataatttaattgataatattagtgttgaaaaattagatactgatatttcacaagcaaatgaaaatgtttttttgaatggaaatggaattgctttagaagtagataatagaggcgctacatgtatttatctagtagatgaaaatggagttaaaacaaaagctacgacttctttagatacagcagatttttcaggttatccaataataggtggacaaaagataagagattttgtaattatatcaaaaaatctagaagaaaacataaactcgatattaggtgttggaaatagacttactattatatctaaaagttcatctactaatctgataagaaagatagtatttgaaacatctaacagcaatccaggagcaatatattcaacagtaagttataaagcagaaagtaacgatttattagtagatagctttcatgaaaatgagtatacaatgagtttagggcaaggaccttttcttgcatatcaagggtgtgcagatcaacaaggagcaaatactatcgttaatgttactaatggatataaccataatagtggacaaaataattattctgtaggagttccatttagttatgtttataactctgtggggggaattggaataggtgatgcatcaacttcaagaagagaatttaagttgcctattataggaaaagataatacagtttcattaggaatggagtggaatggacaaactttaaaaaaaggtgctgaaactgctataggtacaagtgttataactacaacaaatggtgattattattctgggctaaagagttacgcagaagttatgaaagataagggaatatctgcaccagcttcaatacctgatatagcatatgattctagatgggaaagttggggattcgaatttgattttacaatagaaaaaatagttaataaattagatgaacttaaagcgatggggataaaacaaattactctagatgatgggtggtacacttatgctggtgattggaaattaagtcctcaaaagtttccaaatggaaatgcagacatgaaatatcttacagatgaaatccataaaagaggaatgacagctattttatggtggagaccagtagacggagggataaatagcaaattagtatctgaacatccagagtggtttattaagaactcacaagggaatatggttaggttaccagggcctggaggtggaaatggaggaacagcaggatatgcattatgtccaaattcagaaggttcaattcaacatcataaagattttgtaactgtggcattagaagaatggggatttgatggattcaaagaagattatgtatggggaatacctaaatgctatgatagttctcataaacactcaagtttatcagatacattagaaaatcaatataaattctatgaagccatatatgaacagtccatagcgataaatccagatacttttatagaattatgtaattgcggaacacctcaggatttttattcaacaccatatgtgaaccatgcaccaacagcagatccaatttcgagagtacaaacaagaacaagagtgaaagcatttaaagctatatttggagatgattttccagtaacaacagatcataattcagtttggttaccgtcagcattaggtacaggatcagttatgattactaaacatacaacattaagtagttcagatagagaacaatataataaatacttcggacttgcaagagatttagaattagcaaagggagaatttataggaaacttatataaatacggaatagatccattagagtcatatgttataagaaaaggagaagatatttattattcattctacaaagataattctagttattcaggaaatatagaaataaaggggttagacagtaacgccacatatagaattgaagattatgttaacaatagagttattgctagaggagtaaagggaccaacagcgactataaatacaagctttactgataatttattagttagagcaataccagatgatacaccagcagaggttactacatttgatgttggaaataatacaatattatcatcaacagatagtggaaattctaaatatttaaatgctgtttctactacattagaaaagacagcaacaatagatagtttaagtatttatataggaaataattcagaaaatggcaaactacaaattgctatttatgacgataataacgggaaacctggtactaaaaaagcttacgtagaagagtttgttcctactaaaaatagttggaatacaaagaaggttgtaaattctgttacattaccttcagggcaatattggttagttttccaacctgataacgatgtactacaaacaaaaactaatccatcatccatgaaacaaagtgctaacaataatccatataattataatatattaccaaattcatttcctattggaacaggatataatgcttataaaggcgatgtatctttctatgcaacctttaaagaagcaagcagtcaagcaattcctcaaaattcttgggctctaaaatatgtagatagtgaagaaactacaggcgaaaatggaagagctacaaatgcttttgatggtaataataatactatttggcacacaaaatatagtggcggaaacgctgcaccaatgccgcatgagattcaaattgatttaagaggagtatataatataaatcaaattaattatctaccaagacaagatggaggaaccaatggtacaataaaggactatgaagtttatttaagtttagatggagtgaactggggacaacctatatcaaaaggaacctttgaatcaaactctacagaaaaaatagtaaaattcaacgaaacaaaatctaggtatgtaaaacttaaagctctgtcagaaattaataataaacaatttactacagtagctgatttaaaggtatttggatgggagatatccaaaatagaaaaaccattacaaaatgctgaaacttatttgaatataccaacttatgatggattaaatcaaagtactcatccagatgtcaaatattttaaaaatggttggaatggatataaatattggatgataatgactccaaatagaacaggtagctcagttgctgaaaatccttcaatactagcatctgatgatggaataaattgggaggttcctgcaggtgttacaaatcctatagctccaatgccacaagtaggacataattgtgatgttgatatgatatataatgaagcaactgatgagttatgggtgtactgggtagaatcagatgatataacaaaaggatgggttaaattaataaaatcaaaggatggagtaaattggagttctcagcaagtggtagttgatgataatagggcaaaatatagtactttatcaccatctataatattcaaagataataaatactatatgtggtcagttaatacaggaaatagtggttggaacaatcaaagtaataaagttgaattaagagaatcaagtgacggagtaaattggtcaaatccaacagttgtaaacacattagctcaagatggttctcaaatatggcatgtaaatgtagaatatataccatcaaaaaacgaatattgggctatatatccagcatataaaaatggaacaggtagcgataaaacagaattgtattatgcgaaatcaagtgatggagtaaattggacaacttataagaatcctatattatcaaaaggaacatctggtaaatgggatgatatggagatatatagaagttgttttgtgtacgatgaagatacaaatatgataaaggtttggtatggagctgtgagtcaaaatccacaaatatggaaaataggttttactgaaaatgattatgataagtttattgagggtttaacacaataa

SEQ ID NO: 12 - protein, 1320 aa, Clostridium tertium
MKKRILATFITAMCGLGFFSNWTSSNAYNLIDNISVEKLDTDISQANENVFLNGNGIALEVDNRGATCIYLVDENGVKTKATTSLDTADFSGYPIIGGQKIRDFVIISKNLEENINSILGVGNRLTIISKSSSTNLIRKIVFETSNSNPGAIYSTVSYKAESNDLLVDSFHENEYTMSLGQGPFLAYQGCADQQGANTIVNVTNGYNHNSGQNNYSVGVPFSYVYNSVGGIGIGDASTSRREFKLPIIGKDNTVSLGMEWNGQTLKKGAETAIGTSVITTTNGDYYSGLKSYAEVMKDKGISAPASIPDIAYDSRWESWGFEFDFTIEKIVNKLDELKAMGIKQITLDDGWYTYAGDWKLSPQKFPNGNADMKYLTDEIHKRGMTAILWWRPVDGGINSKLVSEHPEWFIKNSQGNMVRLPGPGGGNGGTAGYALCPNSEGSIQHHKDFVTVALEEWGFDGFKEDYVWGIPKCYDSSHKHSSLSDTLENQYKFYEAIYEQSIAINPDTFIELCNCGTPQDFYSTPYVNHAPTADPISRVQTRTRVKAFKAIFGDDFPVTTDHNSVWLPSALGTGSVMITKHTTLSSSDREQYNKYFGLARDLELAKGEFIGNLYKYGIDPLESYVIRKGEDIYYSFYKDNSSYSGNIEIKGLDSNATYRIEDYVNNRVIARGVKGPTATINTSFTDNLLVRAIPDDTPAEVTTFDVGNNTILSSTDSGNSKYLNAVSTTLEKTATIDSLSIYIGNNSENGKLQIAIYDDNNGKPGTKKAYVEEFVPTKNSWNTKKVVNSVTLPSGQYWLVFQPDNDVLQTKTNPSSMKQSANNNPYNYNILPNSFPIGTGYNAYKGDVSFYATFKEASSQAIPQNSWALKYVDSEETTGENGRATNAFDGNNNTIWHTKYSGGNAAPMPHEIQIDLRGVYNINQINYLPRQDGGTNGTIKDYEVYLSLDGVNWGQPISKGTFESNSTEKIVKFNETKSRYVKLKALSEINNKQFTTVADLKVFGWEISKIEKPLQNAETYLNIPTYDGLNQSTHPDVKYFKNGWNGYKYWMIMTPNRTGSSVAENPSILASDDGINWEVPAGVTNPIAPMPQVGHNCDVDMIYNEATDELWVYWVESDDITKGWVKLIKSKDGVNWSSQQVVVDDNRAKYSTLSPSIIFKDNKYYMWSVNTGNSGWNNQSNKVELRESSDGVNWSNPTVVNTLAQDGSQIWHVNVEYIPSKNEYWAIYPAYKNGTGSDKTELYYAKSSDGVNWTTYKNPILSKGTSGKWDDMEIYRSCFVYDEDTNMIKVWYGAVSQNPQIWKIGFTENDYDKFIEGLTQ

SEQ ID NO: 13 - DNA, 3882 bp, Clostridium tertium
tataatttaattgataatattagtgttgaaaaattagatactgatatttcacaagcaaatgaaaatgtttttttgaatggaaatggaattgctttagaagtagataatagaggcgctacatgtatttatctagtagatgaaaatggagttaaaacaaaagctacgacttctttagatacagcagatttttcaggttatccaataataggtggacaaaagataagagattttgtaattatatcaaaaaatctagaagaaaacataaactcgatattaggtgttggaaatagacttactattatatctaaaagttcatctactaatctgataagaaagatagtatttgaaacatctaacagcaatccaggagcaatatattcaacagtaagttataaagcagaaagtaacgatttattagtagatagctttcatgaaaatgagtatacaatgagtttagggcaaggaccttttcttgcatatcaagggtgtgcagatcaacaaggagcaaatactatcgttaatgttactaatggatataaccataatagtggacaaaataattattctgtaggagttccatttagttatgtttataactctgtggggggaattggaataggtgatgcatcaacttcaagaagagaatttaagttgcctattataggaaaagataatacagtttcattaggaatggagtggaatggacaaactttaaaaaaaggtgctgaaactgctataggtacaagtgttataactacaacaaatggtgattattattctgggctaaagagttacgcagaagttatgaaagataagggaatatctgcaccagcttcaatacctgatatagcatatgattctagatgggaaagttggggattcgaatttgattttacaatagaaaaaatagttaataaattagatgaacttaaagcgatggggataaaacaaattactctagatgatgggtggtacacttatgctggtgattggaaattaagtcctcaaaagtttccaaatggaaatgcagacatgaaatatcttacagatgaaatccataaaagaggaatgacagctattttatggtggagaccagtagacggagggataaatagcaaattagtatctgaacatccagagtggtttattaagaactcacaagggaatatggttaggttaccagggcctggaggtggaaatggaggaacagcaggatatgcattatgtccaaattcagaaggttcaattcaacatcataaagattttgtaactgtggcattagaagaatggggatttgatggattcaaagaagattatgtatggggaatacctaaatgctatgatagttctcataaacactcaagtttatcagatacattagaaaatcaatataaattctatgaagccatatatgaacagtccatagcgataaatccagatacttttatagaattatgtaattgcggaacacctcaggatttttattcaacaccatatgtgaaccatgcaccaacagcagatccaatttcgagagtacaaacaagaacaagagtgaaagcatttaaagctatatttggagatgattttccagtaacaacagatcataattcagtttggttaccgtcagcattaggtacaggatcagttatgattactaaacatacaacattaagtagttcagatagagaacaatataataaatacttcggacttgcaagagatttagaattagcaaagggagaatttataggaaacttatataaatacggaatagatccattagagtcatatgttataagaaaaggagaagatatttattattcattctacaaagataattctagttattcaggaaatatagaaataaaggggttagacagtaacgccacatatagaattgaagattatgttaacaatagagttattgctagaggagtaaagggaccaacagcgactataaatacaagctttactgataatttattagttagagcaataccagatgatacaccagcagaggttactacatttgatgttggaaataatacaatattatcatcaacagatagtggaaattctaaatatttaaatgctgtttctactacattagaaaagacagcaacaatagatagtttaagtatttatataggaaataattcagaaaatggcaaactacaaattgctatttatgacgataataacgggaaacctggtactaaaaaagcttacgtagaagagtttgttcctactaaaaatagttggaatacaaagaaggttgtaaattctgttacattaccttcagggcaatattggttagttttccaacctgataacgatgtactacaaacaaaaactaatccatcatccatgaaacaaagtgctaacaataatccatataattataatatattaccaaattcatttcctattggaacaggatataatgcttataaaggcgatgtatctttctatgcaacctttaaagaagcaagcagtcaagcaattcctcaaaattcttgggctctaaaatatgtagatagtgaagaaactacaggcgaaaatggaagagctacaaatgcttttgatggtaataataatactatttggcacacaaaatatagtggcggaaacgctgcaccaatgccgcatgagattcaaattgatttaagaggagtatataatataaatcaaattaattatctaccaagacaagatggaggaaccaatggtacaataaaggactatgaagtttatttaagtttagatggagtgaactggggacaacctatatcaaaaggaacctttgaatcaaactctacagaaaaaatagtaaaattcaacgaaacaaaatctaggtatgtaaaacttaaagctctgtcagaaattaataataaacaatttactacagtagctgatttaaaggtatttggatgggagatatccaaaatagaaaaaccattacaaaatgctgaaacttatttgaatataccaacttatgatggattaaatcaaagtactcatccagatgtcaaatattttaaaaatggttggaatggatataaatattggatgataatgactccaaatagaacaggtagctcagttgctgaaaatccttcaatactagcatctgatgatggaataaattgggaggttcctgcaggtgttacaaatcctatagctccaatgccacaagtaggacataattgtgatgttgatatgatatataatgaagcaactgatgagttatgggtgtactgggtagaatcagatgatataacaaaaggatgggttaaattaataaaatcaaaggatggagtaaattggagttctcagcaagtggtagttgatgataatagggcaaaatatagtactttatcaccatctataatattcaaagataataaatactatatgtggtcagttaatacaggaaatagtggttggaacaatcaaagtaataaagttgaattaagagaatcaagtgacggagtaaattggtcaaatccaacagttgtaaacacattagctcaagatggttctcaaatatggcatgtaaatgtagaatatataccatcaaaaaacgaatattgggctatatatccagcatataaaaatggaacaggtagcgataaaacagaattgtattatgcgaaatcaagtgatggagtaaattggacaacttataagaatcctatattatcaaaaggaacatctggtaaatgggatgatatggagatatatagaagttgttttgtgtacgatgaagatacaaatatgataaaggtttggtatggagctgtgagtcaaaatccacaaatatggaaaataggttttactgaaaatgattatgataagtttattgagggtttaacacaataa

SEQ ID NO: 14 - protein, 1293 aa, Clostridium tertium
YNLIDNISVEKLDTDISQANENVFLNGNGIALEVDNRGATCIYLVDENGVKTKATTSLDTADFSGYPIIGGQKIRDFVIISKNLEENINSILGVGNRLTIISKSSSTNLIRKIVFETSNSNPGAIYSTVSYKAESNDLLVDSFHENEYTMSLGQGPFLAYQGCADQQGANTIVNVTNGYNHNSGQNNYSVGVPFSYVYNSVGGIGIGDASTSRREFKLPIIGKDNTVSLGMEWNGQTLKKGAETAIGTSVITTTNGDYYSGLKSYAEVMKDKGISAPASIPDIAYDSRWESWGFEFDFTIEKIVNKLDELKAMGIKQITLDDGWYTYAGDWKLSPQKFPNGNADMKYLTDEIHKRGMTAILWWRPVDGGINSKLVSEHPEWFIKNSQGNMVRLPGPGGGNGGTAGYALCPNSEGSIQHHKDFVTVALEEWGFDGFKEDYVWGIPKCYDSSHKHSSLSDTLENQYKFYEAIYEQSIAINPDTFIELCNCGTPQDFYSTPYVNHAPTADPISRVQTRTRVKAFKAIFGDDFPVTTDHNSVWLPSALGTGSVMITKHTTLSSSDREQYNKYFGLARDLELAKGEFIGNLYKYGIDPLESYVIRKGEDIYYSFYKDNSSYSGNIEIKGLDSNATYRIEDYVNNRVIARGVKGPTATINTSFTDNLLVRAIPDDTPAEVTTFDVGNNTILSSTDSGNSKYLNAVSTTLEKTATIDSLSIYIGNNSENGKLQIAIYDDNNGKPGTKKAYVEEFVPTKNSWNTKKVVNSVTLPSGQYWLVFQPDNDVLQTKTNPSSMKQSANNNPYNYNILPNSFPIGTGYNAYKGDVSFYATFKEASSQAIPQNSWALKYVDSEETTGENGRATNAFDGNNNTIWHTKYSGGNAAPMPHEIQIDLRGVYNINQINYLPRQDGGTNGTIKDYEVYLSLDGVNWGQPISKGTFESNSTEKIVKFNETKSRYVKLKALSEINNKQFTTVADLKVFGWEISKIEKPLQNAETYLNIPTYDGLNQSTHPDVKYFKNGWNGYKYWMIMTPNRTGSSVAENPSILASDDGINWEVPAGVTNPIAPMPQVGHNCDVDMIYNEATDELWVYWVESDDITKGWVKLIKSKDGVNWSSQQVVVDDNRAKYSTLSPSIIFKDNKYYMWSVNTGNSGWNNQSNKVELRESSDGVNWSNPTVVNTLAQDGSQIWHVNVEYIPSKNEYWAIYPAYKNGTGSDKTELYYAKSSDGVNWTTYKNPILSKGTSGKWDDMEIYRSCFVYDEDTNMIKVWYGAVSQNPQIWKIGFTENDYDKFIEGLTQ

SEQ ID NO: 15 - protein, 1313 aa, Clostridium tertium
MGSSHHHHHHSSGLVPRGSHYNLIDNISVEKLDTDISQANENVFLNGNGIALEVDNRGATCIYLVDENGVKTKATTSLDTADFSGYPIIGGQKIRDFVIISKNLEENINSILGVGNRLTIISKSSSTNLIRKIVFETSNSNPGAIYSTVSYKAESNDLLVDSFHENEYTMSLGQGPFLAYQGCADQQGANTIVNVTNGYNHNSGQNNYSVGVPFSYVYNSVGGIGIGDASTSRREFKLPIIGKDNTVSLGMEWNGQTLKKGAETAIGTSVITTTNGDYYSGLKSYAEVMKDKGISAPASIPDIAYDSRWESWGFEFDFTIEKIVNKLDELKAMGIKQITLDDGWYTYAGDWKLSPQKFPNGNADMKYLTDEIHKRGMTAILWWRPVDGGINSKLVSEHPEWFIKNSQGNMVRLPGPGGGNGGTAGYALCPNSEGSIQHHKDFVTVALEEWGFDGFKEDYVWGIPKCYDSSHKHSSLSDTLENQYKFYEAIYEQSIAINPDTFIELCNCGTPQDFYSTPYVNHAPTADPISRVQTRTRVKAFKAIFGDDFPVTTDHNSVWLPSALGTGSVMITKHTTLSSSDREQYNKYFGLARDLELAKGEFIGNLYKYGIDPLESYVIRKGEDIYYSFYKDNSSYSGNIEIKGLDSNATYRIEDYVNNRVIARGVKGPTATINTSFTDNLLVRAIPDDTPAEVTTFDVGNNTILSSTDSGNSKYLNAVSTTLEKTATIDSLSIYIGNNSENGKLQIAIYDDNNGKPGTKKAYVEEFVPTKNSWNTKKVVNSVTLPSGQYWLVFQPDNDVLQTKTNPSSMKQSANNNPYNYNILPNSFPIGTGYNAYKGDVSFYATFKEASSQAIPQNSWALKYVDSEETTGENGRATNAFDGNNNTIWHTKYSGGNAAPMPHEIQIDLRGVYNINQINYLPRQDGGTNGTIKDYEVYLSLDGVNWGQPISKGTFESNSTEKIVKFNETKSRYVKLKALSEINNKQFTTVADLKVFGWEISKIEKPLQNAETYLNIPTYDGLNQSTHPDVKYFKNGWNGYKYWMIMTPNRTGSSVAENPSILASDDGINWEVPAGVTNPIAPMPQVGHNCDVDMIYNEATDELWVYWVESDDITKGWVKLIKSKDGVNWSSQQVVVDDNRAKYSTLSPSIIFKDNKYYMWSVNTGNSGWNNQSNKVELRESSDGVNWSNPTVVNTLAQDGSQIWHVNVEYIPSKNEYWAIYPAYKNGTGSDKTELYYAKSSDGVNWTTYKNPILSKGTSGKWDDMEIYRSCFVYDEDTNMIKVWYGAVSQNPQIWKIGFTENDYDKFIEGLTQ

SEQ ID NO: 16 - DNA, 1584 bp, Clostridium tertium
tcagggcaatattggttagttttccaacctgataacgatgtactacaaacaaaaactaatccatcatccatgaaacaaagtgctaacaataatccatataattataatatattaccaaattcatttcctattggaacaggatataatgcttataaaggcgatgtatctttctatgcaacctttaaagaagcaagcagtcaagcaattcctcaaaattcttgggctctaaaatatgtagatagtgaagaaactacaggcgaaaatggaagagctacaaatgcttttgatggtaataataatactatttggcacacaaaatatagtggcggaaacgctgcaccaatgccgcatgagattcaaattgatttaagaggagtatataatataaatcaaattaattatctaccaagacaagatggaggaaccaatggtacaataaaggactatgaagtttatttaagtttagatggagtgaactggggacaacctatatcaaaaggaacctttgaatcaaactctacagaaaaaatagtaaaattcaacgaaacaaaatctaggtatgtaaaacttaaagctctgtcagaaattaataataaacaatttactacagtagctgatttaaaggtatttggatgggagatatccaaaatagaaaaaccattacaaaatgctgaaacttatttgaatataccaacttatgatggattaaatcaaagtactcatccagatgtcaaatattttaaaaatggttggaatggatataaatattggatgataatgactccaaatagaacaggtagctcagttgctgaaaatccttcaatactagcatctgatgatggaataaattgggaggttcctgcaggtgttacaaatcctatagctccaatgccacaagtaggacataattgtgatgttgatatgatatataatgaagcaactgatgagttatgggtgtactgggtagaatcagatgatataacaaaaggatgggttaaattaataaaatcaaaggatggagtaaattggagttctcagcaagtggtagttgatgataatagggcaaaatatagtactttatcaccatctataatattcaaagataataaatactatatgtggtcagttaatacaggaaatagtggttggaacaatcaaagtaataaagttgaattaagagaatcaagtgacggagtaaattggtcaaatccaacagttgtaaacacattagctcaagatggttctcaaatatggcatgtaaatgtagaatatataccatcaaaaaacgaatattgggctatatatccagcatataaaaatggaacaggtagcgataaaacagaattgtattatgcgaaatcaagtgatggagtaaattggacaacttataagaatcctatattatcaaaaggaacatctggtaaatgggatgatatggagatatatagaagttgttttgtgtacgatgaagatacaaatatgataaaggtttggtatggagctgtgagtcaaaatccacaaatatggaaaataggttttactgaaaatgattatgataagtttattgagggtttaacacaataa

SEQ ID NO: 17 - protein, 547 aa, Clostridium tertium
MGSSHHHHHHSSGLVPRGSHSGQYWLVFQPDNDVLQTKTNPSSMKQSANNNPYNYNILPNSFPIGTGYNAYKGDVSFYATFKEASSQAIPQNSWALKYVDSEETTGENGRATNAFDGNNNTIWHTKYSGGNAAPMPHEIQIDLRGVYNINQINYLPRQDGGTNGTIKDYEVYLSLDGVNWGQPISKGTFESNSTEKIVKFNETKSRYVKLKALSEINNKQFTTVADLKVFGWEISKIEKPLQNAETYLNIPTYDGLNQSTHPDVKYFKNGWNGYKYWMIMTPNRTGSSVAENPSILASDDGINWEVPAGVTNPIAPMPQVGHNCDVDMIYNEATDELWVYWVESDDITKGWVKLIKSKDGVNWSSQQVVVDDNRAKYSTLSPSIIFKDNKYYMWSVNTGNSGWNNQSNKVELRESSDGVNWSNPTVVNTLAQDGSQIWHVNVEYIPSKNEYWAIYPAYKNGTGSDKTELYYAKSSDGVNWTTYKNPILSKGTSGKWDDMEIYRSCFVYDEDTNMIKVWYGAVSQNPQIWKIGFTENDYDKFIEGLTQ

SEQ ID NO: 18 - DNA, 2958 bp, Clostridium tertium
tataatttaattgataatattagtgttgaaaaattagatactgatatttcacaagcaaatgaaaatgtttttttgaatggaaatggaattgctttagaagtagataatagaggcgctacatgtatttatctagtagatgaaaatggagttaaaacaaaagctacgacttctttagatacagcagatttttcaggttatccaataataggtggacaaaagataagagattttgtaattatatcaaaaaatctagaagaaaacataaactcgatattaggtgttggaaatagacttactattatatctaaaagttcatctactaatctgataagaaagatagtatttgaaacatctaacagcaatccaggagcaatatattcaacagtaagttataaagcagaaagtaacgatttattagtagatagctttcatgaaaatgagtatacaatgagtttagggcaaggaccttttcttgcatatcaagggtgtgcagatcaacaaggagcaaatactatcgttaatgttactaatggatataaccataatagtggacaaaataattattctgtaggagttccatttagttatgtttataactctgtggggggaattggaataggtgatgcatcaacttcaagaagagaatttaagttgcctattataggaaaagataatacagtttcattaggaatggagtggaatggacaaactttaaaaaaaggtgctgaaactgctataggtacaagtgttataactacaacaaatggtgattattattctgggctaaagagttacgcagaagttatgaaagataagggaatatctgcaccagcttcaatacctgatatagcatatgattctagatgggaaagttggggattcgaatttgattttacaatagaaaaaatagttaataaattagatgaacttaaagcgatggggataaaacaaattactctagatgatgggtggtacacttatgctggtgattggaaattaagtcctcaaaagtttccaaatggaaatgcagacatgaaatatcttacagatgaaatccataaaagaggaatgacagctattttatggtggagaccagtagacggagggataaatagcaaattagtatctgaacatccagagtggtttattaagaactcacaagggaatatggttaggttaccagggcctggaggtggaaatggaggaacagcaggatatgcattatgtccaaattcagaaggttcaattcaacatcataaagattttgtaactgtggcattagaagaatggggatttgatggattcaaagaagattatgtatggggaatacctaaatgctatgatagttctcataaacactcaagtttatcagatacattagaaaatcaatataaattctatgaagccatatatgaacagtccatagcgataaatccagatacttttatagaattatgtaattgcggaacacctcaggatttttattcaacaccatatgtgaaccatgcaccaacagcagatccaatttcgagagtacaaacaagaacaagagtgaaagcatttaaagctatatttggagatgattttccagtaacaacagatcataattcagtttggttaccgtcagcattaggtacaggatcagttatgattactaaacatacaacattaagtagttcagatagagaacaatataataaatacttcggacttgcaagagatttagaattagcaaagggagaatttataggaaacttatataaatacggaatagatccattagagtcatatgttataagaaaaggagaagatatttattattcattctacaaagataattctagttattcaggaaatatagaaataaaggggttagacagtaacgccacatatagaattgaagattatgttaacaatagagttattgctagaggagtaaagggaccaacagcgactataaatacaagctttactgataatttattagttagagcaataccagatgatacaccagcagaggttactacatttgatgttggaaataatacaatattatcatcaacagatagtggaaattctaaatatttaaatgctgtttctactacattagaaaagacagcaacaatagatagtttaagtatttatataggaaataattcagaaaatggcaaactacaaattgctatttatgacgataataacgggaaacctggtactaaaaaagcttacgtagaagagtttgttcctactaaaaatagttggaatacaaagaaggttgtaaattctgttacattaccttcagggcaatattggttagttttccaacctgataacgatgtactacaaacaaaaactaatccatcatccatgaaacaaagtgctaacaataatccatataattataatatattaccaaattcatttcctattggaacaggatataatgcttataaaggcgatgtatctttctatgcaacctttaaagaagcaagcagtcaagcaattcctcaaaattcttgggctctaaaatatgtagatagtgaagaaactacaggcgaaaatggaagagctacaaatgcttttgatggtaataataatactatttggcacacaaaatatagtggcggaaacgctgcaccaatgccgcatgagattcaaattgatttaagaggagtatataatataaatcaaattaattatctaccaagacaagatggaggaaccaatggtacaataaaggactatgaagtttatttaagtttagatggagtgaactggggacaacctatatcaaaaggaacctttgaatcaaactctacagaaaaaatagtaaaattcaacgaaacaaaatctaggtatgtaaaacttaaagctctgtcagaaattaataataaacaatttactacagtagctgatttaaaggtatttggatgggagatatccaaaatagaaaaataa

SEQ ID NO: 19 - protein, 1005 aa, Clostridium tertium
MGSSHHHHHHSSGLVPRGSHYNLIDNISVEKLDTDISQANENVFLNGNGIALEVDNRGATCIYLVDENGVKTKATTSLDTADFSGYPIIGGQKIRDFVIISKNLEENINSILGVGNRLTIISKSSSTNLIRKIVFETSNSNPGAIYSTVSYKAESNDLLVDSFHENEYTMSLGQGPFLAYQGCADQQGANTIVNVTNGYNHNSGQNNYSVGVPFSYVYNSVGGIGIGDASTSRREFKLPIIGKDNTVSLGMEWNGQTLKKGAETAIGTSVITTTNGDYYSGLKSYAEVMKDKGISAPASIPDIAYDSRWESWGFEFDFTIEKIVNKLDELKAMGIKQITLDDGWYTYAGDWKLSPQKFPNGNADMKYLTDEIHKRGMTAILWWRPVDGGINSKLVSEHPEWFIKNSQGNMVRLPGPGGGNGGTAGYALCPNSEGSIQHHKDFVTVALEEWGFDGFKEDYVWGIPKCYDSSHKHSSLSDTLENQYKFYEAIYEQSIAINPDTFIELCNCGTPQDFYSTPYVNHAPTADPISRVQTRTRVKAFKAIFGDDFPVTTDHNSVWLPSALGTGSVMITKHTTLSSSDREQYNKYFGLARDLELAKGEFIGNLYKYGIDPLESYVIRKGEDIYYSFYKDNSSYSGNIEIKGLDSNATYRIEDYVNNRVIARGVKGPTATINTSFTDNLLVRAIPDDTPAEVTTFDVGNNTILSSTDSGNSKYLNAVSTTLEKTATIDSLSIYIGNNSENGKLQIAIYDDNNGKPGTKKAYVEEFVPTKNSWNTKKVVNSVTLPSGQYWLVFQPDNDVLQTKTNPSSMKQSANNNPYNYNILPNSFPIGTGYNAYKGDVSFYATFKEASSQAIPQNSWALKYVDSEETTGENGRATNAFDGNNNTIWHTKYSGGNAAPMPHEIQIDLRGVYNINQINYLPRQDGGTNGTIKDYEVYLSLDGVNWGQPISKGTFESNSTEKIVKFNETKSRYVKLKALSEINNKQFTTVADLKVFGWEISKIEK

SEQ ID NO: 20 - DNA, 3786 bp, Robinsoniella peoriensis
gggaacggattagaggtgaaagcctcgccaagggaggtggcacaaataaccggaaacggggtatcggtgacgttttttcaggaagatggcacggtgcagttatcctgtatagaggatgatggcaatactgcttttatgaccaggaactcagaggtctcttatccggtggtgggtggggaggaagtaacagacttttcagactttcaatgtgaagtacaggaaaacgtaaccggagctgcgggagccggcagccggatgacaatcacctccatttccagcggcagggggattcagcggtcggtagtcattgagacggtagatgaggtaaaaggcctgctccatatcagcagttcttatagggcagaagaagaggtagatgcagacgaatttattgacagcagattcagcctggataatccctcagatacagtctggagttacaatggcggcggtgagggggcccagagccgatacgatactctacagaaaatagatctgtcggatggtgaaagcttctatagggagaacttacagaatcaaactgcggcaggtattccggtggcggatatctacgggaaagacgggggtattacggtgggtgatgccagtgtgacccggcgacagctttccactccggtaaacgagaggaatggtaccgcttatgtgtccgtgaaacatccaggtgcagttattacccaaagggaaacagaaatcagccagagctttgtcaatgtacacagaggcgactattattcggggctgcggggttatgccgatggtatgaagcagataggatttaccacactctcccgggaacagattcctgaaagcagctatgatctccgctgggagagctggggatgggaatttgactggacagtggaactgattatcaataagctggacgagttaaaagagatgggaatcaaacagattaccctggatgacggctggtataatgccgcaggagaatgggggctgaacaactggaagcttcctaatggtgctttggacatgcggcatctgactgatgcaattcatgaaagggggatgactgcagtattgtggtggcgtccctgtgacggtggaagggaagacagcgcattatttaaagagcatccagagtattttataaaaaaccaggacggaagctttgggaagctggcaggaccgggacagtggaacagttttctgggaagctgcggttatgcgctgtgtcctttgtcagaaggggcagtacagagccaggttgattttattaaccgtgctatgaatgaatggggatttgatggatttaaaagtgattatgtatggagccttccaaagtgctacagtcaggaccatcaccatgaatacccggaagaatccacagaacagcaggctgtgttctaccgggcagtttatgaggctatgacagacaatgacccgaatgcatttcaccttctatgcaactgcggaacgccacaggattattattctctgccctatgtaacccaggtgcctactgccgatcccacttctgtggatcagacaaggagaagggtaaaggcatataaagcactatgcggtgattatttccctgttacgacagatcataatgaagtctggtatccttcaaccataggaacgggagccatactgattgaaaaacgtgacttgtcaggctgggaagaggaggagtatgcaaaatggcttaaaattgctcaggaaaaccaattgcataaagggacatttattggggatttgtacagttacggatatgacccttatgaaacctatacggtgtataaagacggaatcatgtattatgcattctataaagacggaaaccggtaccgtccgtccggtaacccggatatcgaattaaaagggctggaagacggaaagctgtaccgcatcgtagattatgtaaataatcaggtagttgccacaaatgtaaccagtagcaatgctgtattttcttaccctttcagcgattacttgctggtaaaagcagtagaaatcagcgaaccggatacggatggacctggacctgtaccggatcctgagggggcggtaacagtagaggaaaatgatcctgaactggtatatacaggggattgggtaagggaagaaaatgacggataccatggaggaggagcccgttatacaaaagaagcagaagcttctgtagaattggcattctatggaacaggtgctgcctggtatggacagcacgacgttaactttggtagtgcacggatatatatagacggaacctatgtcaagaccgtatcatgcatgggagaacctggaataaatattaaattgtttgaaatcagcggcttggacttggcttcccacaggattaaaatagaatgtgagacaccggtaattgatattgacaggctgacttacatcaaaggagaagaagttcctgctaaagtaatgacggcggacctccgggctttgactgttatagcaaaccaatacgatatgaacagttttgcagatggcaattacaaagaccagctgggggtatccttagttcgtgccaaccagcttctggcagcggatgatgtaacccagggggctgtaaatgaagaacagaaataccttctgaatgccatgctgaaaataagaaaaaaagttgataagagttggatcgggcttcccggaccaatcccgcaggatatacagacagaaaatatcagcagagataaccttgctaaagtaatatcttatactgggcagttggacagagatgagattattcctgccataaaagaacagctgaacgattcttatgataaggctgtctccatagcagaacgccaggatgcatcccagccggaaatagacagagcgtgggcagagttaatgaatgcagtgcaatatagcagctatatcaggggatcaaaagaggaactgttatcacttctggatgaatacggaaaggtagataccaccgtttataaagacgctgctttatttatagaatccttagaagccgctaaaaaggtgtatcaggatgaaaatgcaatggatggggagatcagtgattgtatcaaacaattgcgagatgcaaaagatcagctacaactaaaggatccggtagatccgccgaaacccgatccggaccccgatccaaagcctgatccaacaccagacccgggaccagatccaaagcccgatccaacacctgacccgacgccagacccaaagcccaatccaacaccgacgcccgatccaacaccagagccagctctaaaaaagccggaacaggtatctggtttgaagtcgaaagcggagactgattatctgacggtttcctggaagaaattgaataatgctgaatcctataaggtgtatatttataaaagcggcaaatggcgcctggctggaaaaactacaaagacatccataaagataaaaaaactggtttcgggaacgaaatacaccgtaaaagttgctgcggtcaataaagcagggcaggggaaatattcatcacaggtgtatacggcagcaaagcccaaaaaagtcaaattaaaatccgtcagcaggtaccgcacatcaaaagtaaagttaaactatggaaaagtaaaagcaggcggatatgaaatatggatgaagaatggaaagggttcttataagaaggcagccaccagtacgaagacaacagccataaagagcggattaaaaaaaggaaaaacatattactttaaagtcagggcttatgttaaaaataaaaatcaggtgatttacggcagcttttccaatataaagaaatacaaaatggtattatga

SEQ ID NO: 21 - protein, 1281 aa, Robinsoniella peoriensis
MGSSHHHHHHSSGLVPRGSHGNGLEVKASPREVAQITGNGVSVTFFQEDGTVQLSCIEDDGNTAFMTRNSEVSYPVVGGEEVTDFSDFQCEVQENVTGAAGAGSRMTITSISSGRGIQRSVVIETVDEVKGLLHISSSYRAEEEVDADEFIDSRFSLDNPSDTVWSYNGGGEGAQSRYDTLQKIDLSDGESFYRENLQNQTAAGIPVADIYGKDGGITVGDASVTRRQLSTPVNERNGTAYVSVKHPGAVITQRETEISQSFVNVHRGDYYSGLRGYADGMKQIGFTTLSREQIPESSYDLRWESWGWEFDWTVELIINKLDELKEMGIKQITLDDGWYNAAGEWGLNNWKLPNGALDMRHLTDAIHERGMTAVLWWRPCDGGREDSALFKEHPEYFIKNQDGSFGKLAGPGQWNSFLGSCGYALCPLSEGAVQSQVDFINRAMNEWGFDGFKSDYVWSLPKCYSQDHHHEYPEESTEQQAVFYRAVYEAMTDNDPNAFHLLCNCGTPQDYYSLPYVTQVPTADPTSVDQTRRRVKAYKALCGDYFPVTTDHNEVWYPSTIGTGAILIEKRDLSGWEEEEYAKWLKIAQENQLHKGTFIGDLYSYGYDPYETYTVYKDGIMYYAFYKDGNRYRPSGNPDIELKGLEDGKLYRIVDYVNNQVVATNVTSSNAVFSYPFSDYLLVKAVEISEPDTDGPGPVPDPEGAVTVEENDPELVYTGDWVREENDGYHGGGARYTKEAEASVELAFYGTGAAWYGQHDVNFGSARIYIDGTYVKTVSCMGEPGINIKLFEISGLDLASHRIKIECETPVIDIDRLTYIKGEEVPAKVMTADLRALTVIANQYDMNSFADGNYKDQLGVSLVRANQLLAADDVTQGAVNEEQKYLLNAMLKIRKKVDKSWIGLPGPIPQDIQTENISRDNLAKVISYTGQLDRDEIIPAIKEQLNDSYDKAVSIAERQDASQPEIDRAWAELMNAVQYSSYIRGSKEELLSLLDEYGKVDTTVYKDAALFIESLEAAKKVYQDENAMDGEISDCIKQLRDAKDQLQLKDPVDPPKPDPDPDPKPDPTPDPGPDPKPDPTPDPTPDPKPNPTPTPDPTPEPALKKPEQVSGLKSKAETDYLTVSWKKLNNAESYKVYIYKSGKWRLAGKTTKTSIKIKKLVSGTKYTVKVAAVNKAGQGKYSSQVYTAAKPKKVKLKSVSRYRTSKVKLNYGKVKAGGYEIWMKNGKGSYKKAATSTKTTAIKSGLKKGKTYYFKVRAYVKNKNQVIYGSFSNIKKYKMVL

SEQ ID NO: 22 - DNA, 1347 bp, Ruthenibacterium lactatiformans
gaagaaaccgatttgcttgtaaacggaggttttgagaccggcgacagcaccggatggaattggttcaataacgccgttgttgacagcgctgctccgcatagcggaaactattgtgctaaagtagccaaaaacagcagttatgagcaagttgttacggtatctccggatacgaaatatgttttaacagggtgggcaaaatctgagggcagttccgttatgacgctgggcgtaaaaaattacggtgggcaggaaactttttcggctacgctttcagccgactatcagcagctggcggttactttcacaaccgggcccaatgcgcaaacagcgactatatatggatatcgacagaatagtggttccggtgcaggctatttcgacgatgtagaacttacagcggtgcaagattttgctccatatcagccgttggcaaatgccatagcgcctcaagcaattcctacctatgacggcgccaaccagcctacacatccctcggtggtgaaatttgaacagccttggaatggttatctgtattggatggcaatgacaccttatcccttcaatgatgggagctacgaaaacccatcgattgttgcgtcaaacgatggagaaaattggattgtgccagaaggggtctcgaatcctttggccggcacgccaagtccgggccacaattgtgacgtggatcttgtatatgttccagcctcggatgaattgcggatgtactacgtagaggcagatgatatcatcagctcaagggtaaaaatgataagttcccgtgacggtgtacactggagcgagccgcaggtcgtaatgcaggatctggtaaggaaatacagtattctatcgccgtctattgagattctgccagatggcacctatatgatgtggtatgtggatacggggaatgcaggatggaatagccagaataaccaagtaaaatatcgtacatctgcggatggaatcaaatggtcaggcgcagtcacctgtacggattttgtacaacctggatatcaaatatggcacatcgatgtacattatgacacatcaagcggagcttactatgcagtttatccggcttatccgaatggcaccgattgcgaccactgcaatttgtttttcgcagtgaatcggacaggaaaacagtgggaaacttttagccggccaattttgaagccgtcaacggaaggcggctgggatgatttctgcatttaccggtcctctatgctgattgacgacggaatgttgaaagtgtggtacggagcaaaaaagcaagaggattcttcctggcatactgggctaaccatgcgtgatttttctgaatttatgaaaatattggaacgctaa

SEQ ID NO: 23 - protein, 468 aa, Ruthenibacterium lactatiformans
MGSSHHHHHHSSGLVPRGSHEETDLLVNGGFETGDSTGWNWFNNAVVDSAAPHSGNYCAKVAKNSSYEQVVTVSPDTKYVLTGWAKSEGSSVMTLGVKNYGGQETFSATLSADYQQLAVTFTTGPNAQTATIYGYRQNSGSGAGYFDDVELTAVQDFAPYQPLANAIAPQAIPTYDGANQPTHPSVVKFEQPWNGYLYWMAMTPYPFNDGSYENPSIVASNDGENWIVPEGVSNPLAGTPSPGHNCDVDLVYVPASDELRMYYVEADDIISSRVKMISSRDGVHWSEPQVVMQDLVRKYSILSPSIEILPDGTYMMWYVDTGNAGWNSQNNQVKYRTSADGIKWSGAVTCTDFVQPGYQIWHIDVHYDTSSGAYYAVYPAYPNGTDCDHCNLFFAVNRTGKQWETFSRPILKPSTEGGWDDFCIYRSSMLIDDGMLKVWYGAKKQEDSSWHTGLTMRDFSEFMKILER

SEQ ID NO: 24 - DNA, 5277 bp, Robinsoniella peoriensis
tcaccattgagcgctgcggcagaaagtggcacaggaaccagattagtgaaagggcaaacggggtatttgacagaggaacaggctatccggaaccaggagcagacaaccgaagaaagggagcagaagttaaccggggaagagacagcagaggttttgatggaaggtacaaaagacagcgggattgtacagacagaagaagtacagacaaaagaaatgcagacagaagatgcgcagacagaagaagtacagacagaagaaatgcagacagaagatgcgcagacaaaagaagtacagacagaagaaatgcagacagaagatgcgcagacagaagaagtacagacaaaagaagaaccggcagaagaaacacacatgaaagaaatacagacgcaagggacaaagaaagcgtcagataggaacggaaaggcaagggtaactgaaattctggaagatgcccaggatccagcaaaccggattgtgtatctgtcagacctgcaatggaagtcagaaaatcatacagtagatagcgagctgcctaccagaaaggataagtcctttggcggcggaaaaattacgctaaaagtggatggaacggtaacagaatttgataaggggattggaacacagacagattccaccattgtgtacgatctggagggaaagggatatacaaagtttgaaacttacgtgggtgtagactacagccagaaagaaaacattccgggggaagtctgcgacgtaaaattcagggtgaaaattgatgacaagattgtatcagaaaccggtgtactggatccgctttcgaatgcggttaagatttctgttaacatacccgatacagccaaaactttaacattatacgcggataaagtaacggaaacttggtctgatcacgccaattgggcagatgcaaaattttatcaggcactgccggaacccgaaaatgttgcattcaaaaaaacggtagtgacacgaaagacatcagataattcggaggctcctgttaatccggattcagcagttaacagttctaaggctgttgacggtgttattgacagctccagttattttgattttggagatcaggcaaatagcggagccgtaagggagtcactctatatggaggtagatttaaaagggagctatttactgtccgatatacaactgtggagatactggaaagatggcagaacttatgcagctactgcaattgtagtagctgaggatgagaactttgaaaatgcagcagttatctataactcggatacgacgggagaaatacatcacctgggagcaggaagtgatatgctctatgcagaaacagaaagtggcaagacatttccggtaccggaaaatacaaaagcaaggtatatcagagtttatacatatggtgttaatgggacatcaggcgtaacaaatcacattgtagaattaaaggtgaatgcttacgtatttggagatgaaatcttaccggaaaagccggatgacagcaagattttcccaaatgcagttaatccgctgaagctacagggaccgggcacgaatgatcaggtaacccacccggatgttacggtgtttgatgagccgtggaatgggtataaatactggatggcatatacaccgaataaaccgggaagttcctattttgaaaatccctgtatagctgcatccaacgatggcgtaaactgggagtttcctgcccagaaccctgtacagccgcgctatgacagtgaaatagaaaatcaaaatgaacataactgtgataccgatattgtatatgacccggtaaatgaccggttgattatgtactgggaatgggcacaggatgaggcggttaatggtaaaacacatcgttctgaaatcagataccgtgtttcttatgatgggattaactggggagtggaagacaaaactggtgttttgatgactggaccaacggatcatggctgcgccattgccacagaaggcgaaagatattcagacctttctccaaccgtagtatatgataaaacagaaaaaatctacaaaatgtgggcaaatgatgccggagatgtaggatatgaaaacaaacagaataacaaagtatggtatcggacatcccaagacgggatcagcaattggtcggataagacttacgtggagaattttcttggagtaaatgaagacgggctgcagatgtatccatggcaccaggatatccagtgggtagaggaatttcaggaatattgggcacttcagcaggcatttccggcaggaagcggaccggataattcttccctgcgtttctcgaaatccaaagatggtcttcattgggagccggtatctgaaaaagctttaattacagtaggggcacccgggacctgggatgcaggacagatataccgttctactttctggtatgagccaggtggggcaaaaggaaacggaacattccatatctggtatgctgcattggcggaaggccagtctcactgggatataggatatacatctgcaaactatgcagatgccatgtacaaattaacgggaagcagaccggaagtggaaaaaagaatagaggtaaataatgaaaatcctctgctgattatgccgctttacggaaagtcttacagtgaatcaggaagtaccctggattggggagatgatctggtttcacgctggaaacaggttccggaagatttaaaagaaaacgcagttattgaaattcatctgggtggcaagattggcttaaatgaaagtgattcccacacggcaaaagcgttttatgagcagcagctggcaattgcccaggaaaataacatcccggtaatgatggtggtagctacggcaggccagcagaactactggacgggaacagcgaatctggatgctgagtggattgaccggatgttcaagcagcacagtgtgttaaaaggaattatgtccactgaaaattattggactgactacaataaggttgctactatgggtgccgattatctgcgggttgcagctgaaaacggcggatattttgtatggagcgagcaccaggagggtgttattgaaaatgtaatagcaaatgagaaatttaatgaagcattgaaactttacggtaataattttattttcacctggaagaacacgcctgccggtactaactccaatgcaggaacagccagctatatgcagggcctctggctaaccggaatttgtgcacaatggggcggtctggctgatacctggaaatggtatgaaaaaggatttggtaaattatttgatggtcagtattcttataatccgggtggggaagaagcaagaccggttgcaaccgaaccggaagcactgcttggtatcgaaatgatgagtatctatacaaatggcggatgtgtctacaactttgagcacccggcgtatgtatatggttcttataaccagaattcaccttgctttgaaaatgtaattgcagagttcatgcgctatgcgattaagaatccggcaccaggtaaagaggaagtgcttgctgatacaaaagcagtgttctatggaaaattaagttctttaaagagtgcaggaaacttactgcaaaaaggtttgaactgggaagatgccacactgccaacccagactacgggtcgatatggattaatacctgcagtcccggaggcagtagatgaaaaaactgtaaaagcagtattcggcgatattgagatattgaatcaatccagtgcacagcttgcgaataaagatgcgaaaaaagcatattttgaagaaaaatatccggaacagtataccggtacggcatttggacagctattgaatgatacctggtatttatacaacagtaatgtgaatgtggatggggtgcaaaatgcaaaacttccgttagaaggtaataaatccgtagatattacaatgacaccgcatacttatgtgatcctggatgatcaggatggtgagcttcagattaaactgaacaattatcgtgtggataaagacagtatctgggaaggatacggcaccacggtgacggaccgctgggatacggaccacaataccaaacttcaggactggatacgggatgagtatattccaaatccggacgatgataccttcagagatacaacctttgaactggttggactggaaagtgagccggaggtaaatgtaactaatggcttaaaggatcagtatcaggaaccggttgtggaatatgatgccgctgcaggtacggctatgattactgtatccggaaatggctgggtagatctgacaattgacacgaacacggcagaagtaccccaggttgataaagcaaagttaaattccaaaatagcagaagctaaagggatcagacaggggaactatacggatgaatcctacaaagctcttcaggaagagattggaaaatcccaggcggtatcaaacaaaacagatgccacacaggaggaagtaaatgcacagttaagcaggttagaaagtgcaatagccagattaaaagaaaaaccggcggtggtatccaaaacggcattaaatgcaaaaatagctgaggcaaaagggatcagacaaggaaactatacggatgaatcctacaaagccctgcaaaatgcaatagtaaaagctcaggagttatcaaacaaaacagatgccacacagcagcaggtaaatgatctggtatcagcattaacaaatgcaattaaaaatttaaaaatagatgcagataagctggcagcagagtcagcaaagaaagtagcggcagttaaggttgccgtaaaagcagtatcctataaatcaaaagagattaaattatcctggaaaacggtagcagatgcggacggatatgtaatccgtgtaaagacaggcaaaaagtggagtacggagaagaccattaagaacaaccgcataatcacttatacttataagaaaggtactcccggtaagaaatatgtatttgaagtaaaagcttttaagaaagtaaatggaaagacgacctatagtaaatacaaaacagccactaaaaaagttgtgccgcaaacggtgaccgcaaaggcaaaagcttctaaaaataatgtagtggtaaaatggaacaaagtgtctggcgcatccggatatgttgttatgaaaaagaaagggaaaacatgggtaaaggctgcgcaggtaaatgcaaagaaactatactttacggataagaaggtcaaaaaaggaaaagtatattcatacaaagtaaaggcttacaaagtatataaaggtaaaaaagtatatggaagctatagcaagtctgtaaatgttaaaacaaagtcataa

SEQ ID NO: 25 - protein, 1778 aa, Robinsoniella peoriensis
MGSSHHHHHHSSGLVPRGSHSPLSAAAESGTGTRLVKGQTGYLTEEQAIRNQEQTTEEREQKLTGEETAEVLMEGTKDSGIVQTEEVQTKEMQTEDAQTEEVQTEEMQTEDAQTKEVQTEEMQTEDAQTEEVQTKEEPAEETHMKEIQTQGTKKASDRNGKARVTEILEDAQDPANRIVYLSDLQWKSENHTVDSELPTRKDKSFGGGKITLKVDGTVTEFDKGIGTQTDSTIVYDLEGKGYTKFETYVGVDYSQKENIPGEVCDVKFRVKIDDKIVSETGVLDPLSNAVKISVNIPDTAKTLTLYADKVTETWSDHANWADAKFYQALPEPENVAFKKTVVTRKTSDNSEAPVNPDSAVNSSKAVDGVIDSSSYFDFGDQANSGAVRESLYMEVDLKGSYLLSDIQLWRYWKDGRTYAATAIVVAEDENFENAAVIYNSDTTGEIHHLGAGSDMLYAETESGKTFPVPENTKARYIRVYTYGVNGTSGVTNHIVELKVNAYVFGDEILPEKPDDSKIFPNAVNPLKLQGPGTNDQVTHPDVTVFDEPWNGYKYWMAYTPNKPGSSYFENPCIAASNDGVNWEFPAQNPVQPRYDSEIENQNEHNCDTDIVYDPVNDRLIMYWEWAQDEAVNGKTHRSEIRYRVSYDGINWGVEDKTGVLMTGPTDHGCAIATEGERYSDLSPTVVYDKTEKIYKMWANDAGDVGYENKQNNKVWYRTSQDGISNWSDKTYVENFLGVNEDGLQMYPWHQDIQWVEEFQEYWALQQAFPAGSGPDNSSLRFSKSKDGLHWEPVSEKALITVGAPGTWDAGQIYRSTFWYEPGGAKGNGTFHIWYAALAEGQSHWDIGYTSANYADAMYKLTGSRPEVEKRIEVNNENPLLIMPLYGKSYSESGSTLDWGDDLVSRWKQVPEDLKENAVIEIHLGGKIGLNESDSHTAKAFYEQQLAIAQENNIPVMMVVATAGQQNYWTGTANLDAEWIDRMFKQHSVLKGIMSTENYWTDYNKVATMGADYLRVAAENGGYFVWSEHQEGVIENVIANEKFNEALKLYGNNFIFTWKNTPAGTNSNAGTASYMQGLWLTGICAQWGGLADTWKWYEKGFGKLFDGQYSYNPGGEEARPVATEPEALLGIEMMSIYTNGGCVYNFEHPAYVYGSYNQNSPCFENVIAEFMRYAIKNPAPGKEEVLADTKAVFYGKLSSLKSAGNLLQKGLNWEDATLPTQTTGRYGLIPAVPEAVDEKTVKAVFGDIEILNQSSAQLANKDAKKAYFEEKYPEQYTGTAFGQLLNDTWYLYNSNVNVDGVQNAKLPLEGNKSVDITMTPHTYVILDDQDGELQIKLNNYRVDKDSIWEGYGTTVTDRWDTDHNTKLQDWIRDEYIPNPDDDTFRDTTFELVGLESEPEVNVTNGLKDQYQEPVVEYDAAAGTAMITVSGNGWVDLTIDTNTAEVPQVDKAKLNSKIAEAKGIRQGNYTDESYKALQEEIGKSQAVSNKTDATQEEVNAQLSRLESAIARLKEKPAVVSKTALNAKIAEAKGIRQGNYTDESYKALQNAIVKAQELSNKTDATQQQVNDLVSALTNAIKNLKIDADKLAAESAKKVAAVKVAVKAVSYKSKEIKLSWKTVADADGYVIRVKTGKKWSTEKTIKNNRIITYTYKKGTPGKKYVFEVKAFKKVNGKTTYSKYKTATKKVVPQTVTAKAKASKNNVVVKWNKVSGASGYVVMKKKGKTWVKAAQVNAKKLYFTDKKVKKGKVYSYKVKAYKVYKGKKVYGSYSKSVNVKTKS

SEQ ID NO: 26 - DNA, 7899 bp, Robinsoniella peoriensis
gctgagactgcaacagaagaaaatgcggcgctggaaaaaacagttacattgcataagagcgatggaacagaactgccggaggattatcgaaatccccaaagaccagctaccatggcggtagatggtattattgacgatacaggagagtacaactattgcgatttcggtaaagacggtgataaagcagccctgtatatgcaggtggaccttggaggtctgtatgatttaagcagagtcaatatgtggagatactggaaagacagcagaacttacgatgcaacagtaattaccacatctgagagcggcgatttcacagatgaagcagtcatatataattcagacaggtcgaatgtacatggatttggggcaggaggagatgaacgctacgcagagactgcctccggacatgaattcccagtaccggacggtacaaaggcacaggcagtacgcgtatatgtatttggcagccaaaacggtactacaaaccacatcaatgaattgcaggtctggggaactccccatacagagaatccggatgtaaattcttatcaggtgacaattccacagggaaatggatatcaggtaataccttatgaaaatgacccgacgacagtggaagaaggcggttctttccgttttcaggtactgattgactccgataatggttacagcgcaaccagtgcggtaaaagcaaatggagtaagtctggaggcagttgacagtgtttataccattgagaacattactgaagatcaggtaatcaccattgaaggcgtacataaagcacagtatgaagtgaaattcccggaaaatccacagggatacagtgttgagattcagaatgaaggaagtacaacggtagactataatggttctgtcagttttaagcttattatagacgaagcttataatgaatccgtaccggttgtaaaagcaaacggcggtgcagctttgggaaaagatgagctcggtgtatatacaattgcaaatatccaggacgatattacggttacagttgagggtatccaggaaaataccgtagtaaagacaaaaacaatgtacttgtctgatatggattggaagagtgctgcaaatgcagtaggtgcaacaggagaaaaagacactccaacaaaggacctgaatcatttacagcagcagatgaaattattggtaaacggagcagagaagtcttttgataaaggaattggagttcagacggattcttctatcgtttatgatctggaagacaaaggctacacttctttccacaccctggcaggcgttgattattcagcaatggaatatgtagacggagaaggctgtgatatccagtttaaagtatatctggatgatgtcgtagtatttgacagcggagtagttgatgcatctgatgaggctcaggaagttaatgttgctataacatcagagaataaagaactaaaactggaagctaaaatggttaaagagccttataatgactggggaaactgggcagatgccagctttgaaatggcttatcccgaaccgtctaatgtggctttaaataaaacagttaccgttaagaaaacagcggataactcagactctgaagtaaattccagcagaccgggatcaatggctgtagatggaatcattggacctacatcagattctaactattgtgattttggacaggatggggataatacttcccgttatctgcaggtagatttaggggatgtttatgaacttacccagattaatatgtttagatactgggcagatggcagagtatataatggtactgtaattgcagtttccgaaaacgcagactttagtaatccaacttttatttataattcagataaagcagacaaacacggacttggcgcaggcagtgatgacacttatggagaaacccagagtggaaaattattcgaagttccggcgggaaccatgggacagtatgtccgtgtgtatatggctggttccaacaaaggtacaacgaaccatatcgctgaattacaggtaatgggttataatttcaatacagaaccaaaaccatatgaagcaaatgcatttgaaaatgcagaagtttatttagatatgccaactcatttccaggatctggattccaataaaaacgacgatggaagcttaaagcacattggcggacaggtgacacatcctgatatccaggtatttgaccaaccgtggaacggttataaatactggatgatttacacaccaaatacaatgatcacttcccagtatgaaaatccatatatcgtagcatctgaagatggacagacatgggtagaaccggaagggatttccaatccaattgaaccagaaccgccatcaaccagatttcataactgtgatgcagatctgttatacgactctgtcaatgaccgtttacttgcttactggaactgggcagatgacggcggcggaattgatgacgaattaaaagatcagaactgtcagattcgtctgagaatttcttatgatggaattaactggggagttccttacgacaaagacggcaatattgccacaacagctgatactgtagtaagaatggaaacaggagataaggatttcattcctgcaatcagcgaaaaagaccgttatggtatgctttccccaacatttacctatgacgatttccgcggcatatatacaatgtgggcacaaaactcgggtgatgcgggatacaaccagtccggaaagttcatcgaaatgagatggtctgaggatggaataaactggtctgaaccacaaaaagtgaataatttccttggaaaagatgagaatggcagacagctttggccatggcatcaggatattcagtatatccctgagctacaggaatattggggactgtcccagtgtttctctacatctaatcccgatggatccgtattatacctgaccaagtccagagatggtgtcaactgggagcaggcaggaacacagccggtattaagggcaggaaaatcaggtacctgggatgatttccagatttaccgttctaccttctattatgataatcagtcagacagccctactggtgggaaatttagaatctggtacagtgcactgcaggcaaatacttcaggcaagaccgttttggctcctgatggaacagtgtctcttcaggttggaagccaggataccaggatctggcgtatcgggtatacagaaaatgactacatggaagtcatgaaagctctgacccagaataaaaactatgaagaaccggaattagtagacgcagtttccttaaatctgtcaatggataaaacaagcatttcagtaggtgaagaagcaacggtaagcactgctttcgtaccggaaaatgctaccgaccgcattgtaaaatatacatctcaggatccggaaattgcagttattgatccaacaggcattgttacaggggttaaggatggaaccacaactattgttgcagaaacaaaatcgggcgcaaaaggtgaattatccgtaacggttggtgagcttcaaagaggtgaaattcgatttgaggtcagcaatgaccatccgatgtatctggagaattactattggagtgatgatgcaccaaaaaaagacggcttagacgcaaacaagaactactatggggatgaacgtgtcgacagtccggtaatgctgtataataccgttcctgaagaattgaaggataatacagtcatcctgttaattgcagagagaagcttaaacagcacagatgcagtaagggattggattaaaaagaatgttgaattatgtaatgaaaataagattccatgtgcagttcagattgcaaatggagaaacaaatgtaaatacaaccattccattatcgttctggaatgagctggcaacgaacaatgaatacctggttggatttaatgcagccgagatgtataaccgttttgcaggtgacaaccgcagctatgttatggatatgatccgtttaggggtatcccacggcgtatgcatgatgtggaccgataccaatatttttggtacaaacggtgtgttgtatgactggctgactcaggatgaaaaactgtccggtcttatgcgggaatacaaagagtatatctctctgatgacaaaagagtcttacggcagtgaggcagcaaatacagatgctctgtttaagggcctgtggatgacagactactgcgagaactggggaatcgcctccgactggtggcattggcagttagacagcaatggagcactctttgatgcaggcagcggcggagatgcatggaaacagtgtctgacatggccggaaaatatgtatacgcaggacgttgtgcgtgcagtaagccagggtgcaacctgctttaaatcagaagcacagtggtattcaaatgctacaaaaggcatgcgtacaccgacatatcagtattccatgattccgttcctggagaaactggtaagcaaagaggtaaaaattcctacaaaagaagagatgctggaaagaacaaaagcaattgttgtaggggcagaaaactggaataactttaattataatactacttattcaaatctgtatccaagcacaggacaatatggaatcgtaccttatgtaccttcaaattgtccggaagaagaactggcaggctatgatctggtagtaagggaaaaccttggcaaagcaggactgaagtctgcacttgatacggtatacccggttcagaaatcagaaggaaccgcatactgtgaaacctttggagatacctggtactggatgaattcctcggaagacaaaaacgtaagccagtacactgaatttacaaccgcaatcaatggagctgaaagtgtaaagatagccggcgaaccccatgtatttggtattataaaagaaaatccgggatctttaaatgtatacttaagcaactaccgcctggataaaacagaactctgggatggtacaatccccggaggattaagcgatcagggctgctataattatgtatggcagatgtgtgagcgcatgaagaatggaacagggctggatacacagcttcgtgacaccgttattaccgttaaaaatgcagtagaaccgaaagtaaactttgtaacagaatctccggcagacagaagttttgcagaagataattatgtaagaccatacaaatatacggttgcacaaaaagaaggcacaaccgatgaatgggtgattacggtcagccacaatggtattgtggaattcaatattgtaacaggcgatgaaaaagtgccggcaacaagtgtggaattatcaactgataaagttgatgtaatccgtaaccggacagcagttgtaaaggcaacggtattgccgcagaatgcaggaaataaacagttaacatggacaatcgccgatcctgagattgcttctgtagacaacaaaggaaccgtaaccggactaaaagaaggaaaaaccgtattacgtgcagctatttctggcagtgtttataaagaatgcgaagtaaatgtaattgaccgaaaagtaacggaagtaaacttaaacaaaacagagttgtctcttagtgcaggggattctgcgaaactggaagcatccatagcaccggaagacccgtctgacagcagcattacctggacttccacaaatgaaaatgttgcaacggttgcatcaaacggtaccgttacagctcataaagcaggtgtagctcagattatcgcccagtctgcttaccaggcaaagggtatcgcaactgttaccgttaattatgcggcttccgtaaaattagaccgtacaggaatgacggctacagccaacagcgaacagtctaaatcaggtggagaaggacctgcttccaacgtactggacggtaagcaggacacaatgtggcatacaagctggacagataaacctgaattacatcctcactggattaaaattgatttaaacggaacaaaaacaattaacaaatttgcttatacaccaagaaccggagcatctaacggaacaatttataattatgttctgattatcaccgatctggaaggaaatgaaaaacaggttgcaaagggcgtatgggcagcaaatgcagatgtaaaatatgctgaatttgacgcagttgaagctacggcgatcaagctgcaggtagacggcaacgatgacaaggcatcaaaaggaggatatggttccgcggcagaaatcaatatttttgaagtggcacagaaaccttccgcaaatgagcttgccgaaaatattaaagtaattgcacctgtaaaagcagaagatacaaaagtatctatcccagtcattactggatttgatatcgtaatcagtaattccagcaatccggacgtaattggtattgatggcagcatcaccagaccggaaaatgatacagttgtaactttaacattaaaagtaaaagaaacagacgcaaagagtgtaaaggcagcaggaactgaagcaaccacaaatgtggatgttctggttaccggtacaaagacatctgatgtagaggcagaaagcgttacgttagatcagacatcagctgatttaacagttggaggcgaacttttattaaatgccgttgtgaagccggacattgcaactaataaggctgttacctggagctcagataagccgggaaccgctactgttgaaaatggcagggtaaaagcgttagcggcaggagaggcacgtattacagcagcaactgcaaatggaaagacagcagactgcgtcattaacgtaaaggaaaaagaggagccggaagtaattctcccggcagaagtgcgcttaaacattccatcagctgaatttacagtaggagatcagattcagttaactgcttctgtactgccggcaaatgcagcagataagacaattacctggaaatcagacaaacctgaagtggcaactgtcgcaaatggatgggtaaaaggtattgcagccggaactgctaagattacagcaacatcagtcaatggaaaaacggctgtatgtgtgatcacagtcaaagcacagccacagaatctaccaaccggtgtttcactgaacaagaaaacagcaagtgtaaaactgaataaaacccttacactttccgctgtagtacagccttccaatgcggataataagaccgttaaatggacgtctgacaatacgtatgttgcaacagttgagaatggagtcgtaaaagcagttaatgcaggaacagccagaatcactgcagctaccgtaaacggacataaggcaacttgtactataacagtaccgggcacaaagatttccaaggcaaaagtaagccttgcatcatcaaaaacacatacaggcaaagcattaaaaccatctgtaaaagtaacttacggtaagaatacattaaagaaaaatactgattataccgtatcttacaaaaataatataaatcctggaactgcatctgttacgattacgggcaagggtaaatattatggtaccatcaacaaaacttttgcaatcaaggcagcagaaggaaagacctacacggttggtaaaggaaaatataaagttactgatgcttcagcaaagaacaaaacagtaacctttatggctcctgtaaagaagacctacagctcattcagcgtaccttctaaggttaagatcgggaatgatacttacaaagtaactgcagttgcaaaaaatgcattcaaaaagaatacaaagcttacaaagttaaccattggttcgaatgtaaaaacaattggttcttatgcattttatggcgcttcccaattaaaaacgcttaccttaaaaactaccggacttaacagtgtaggcaagaatgcatttaagaaaacaaatgcaaagctgactgtaaaggttccaaagtcaaaattagcagattataagaagctgttaaaaggaaaaggattatctggcaaggcaaaaattcagaaataa

SEQ ID NO: 27 - protein, 2652 aa, Robinsoniella peoriensis
MGSSHHHHHHSSGLVPRGSHAETATEENAALEKTVTLHKSDGTELPEDYRNPQRPATMAVDGIIDDTGEYNYCDFGKDGDKAALYMQVDLGGLYDLSRVNMWRYWKDSRTYDATVITTSESGDFTDEAVIYNSDRSNVHGFGAGGDERYAETASGHEFPVPDGTKAQAVRVYVFGSQNGTTNHINELQVWGTPHTENPDVNSYQVTIPQGNGYQVIPYENDPTTVEEGGSFRFQVLIDSDNGYSATSAVKANGVSLEAVDSVYTIENITEDQVITIEGVHKAQYEVKFPENPQGYSVEIQNEGSTTVDYNGSVSFKLIIDEAYNESVPVVKANGGAALGKDELGVYTIANIQDDITVTVEGIQENTVVKTKTMYLSDMDWKSAANAVGATGEKDTPTKDLNHLQQQMKLLVNGAEKSFDKGIGVQTDSSIVYDLEDKGYTSFHTLAGVDYSAMEYVDGEGCDIQFKVYLDDVVVFDSGVVDASDEAQEVNVAITSENKELKLEAKMVKEPYNDWGNWADASFEMAYPEPSNVALNKTVTVKKTADNSDSEVNSSRPGSMAVDGIIGPTSDSNYCDFGQDGDNTSRYLQVDLGDVYELTQINMFRYWADGRVYNGTVIAVSENADFSNPTFIYNSDKADKHGLGAGSDDTYGETQSGKLFEVPAGTMGQYVRVYMAGSNKGTTNHIAELQVMGYNFNTEPKPYEANAFENAEVYLDMPTHFQDLDSNKNDDGSLKHIGGQVTHPDIQVFDQPWNGYKYWMIYTPNTMITSQYENPYIVASEDGQTWVEPEGISNPIEPEPPSTRFHNCDADLLYDSVNDRLLAYWNWADDGGGIDDELKDQNCQIRLRISYDGINWGVPYDKDGNIATTADTVVRMETGDKDFIPAISEKDRYGMLSPTFTYDDFRGIYTMWAQNSGDAGYNQSGKFIEMRWSEDGINWSEPQKVNNFLGKDENGRQLWPWHQDIQYIPELQEYWGLSQCFSTSNPDGSVLYLTKSRDGVNWEQAGTQPVLRAGKSGTWDDFQIYRSTFYYDNQSDSPTGGKFRIWYSALQANTSGKTVLAPDGTVSLQVGSQDTRIWRIGYTENDYMEVMKALTQNKNYEEPELVDAVSLNLSMDKTSISVGEEATVSTAFVPENATDRIVKYTSQDPEIAVIDPTGIVTGVKDGTTTIVAETKSGAKGELSVTVGELQRGEIRFEVSNDHPMYLENYYWSDDAPKKDGLDANKNYYGDERVDSPVMLYNTVPEELKDNTVILLIAERSLNSTDAVRDWIKKNVELCNENKIPCAVQIANGETNVNTTIPLSFWNELATNNEYLVGFNAAEMYNRFAGDNRSYVMDMIRLGVSHGVCMMWTDTNIFGTNGVLYDWLTQDEKLSGLMREYKEYISLMTKESYGSEAANTDALFKGLWMTDYCENWGIASDWWHWQLDSNGALFDAGSGGDAWKQCLTWPENMYTQDVVRAVSQGATCFKSEAQWYSNATKGMRTPTYQYSMIPFLEKLVSKEVKIPTKEEMLERTKAIVVGAENWNNFNYNTTYSNLYPSTGQYGIVPYVPSNCPEEELAGYDLVVRENLGKAGLKSALDTVYPVQKSEGTAYCETFGDTWYWMNSSEDKNVSQYTEFTTAINGAESVKIAGEPHVFGIIKENPGSLNVYLSNYRLDKTELWDGTIPGGLSDQGCYNYVWQMCERMKNGTGLDTQLRDTVITVKNAVEPKVNFVTESPADRSFAEDNYVRPYKYTVAQKEGTTDEWVITVSHNGIVEFNIVTGDEKVPATSVELSTDKVDVIRNRTAVVKATVLPQNAGNKQLTWTIADPEIASVDNKGTVTGLKEGKTVLRAAISGSVYKECEVNVIDRKVTEVNLNKTELSLSAGDSAKLEASIAPEDPSDSSITWTSTNENVATVASNGTVTAHKAGVAQIIAQSAYQAKGIATVTVNYAASVKLDRTGMTATANSEQSKSGGEGPASNVLDGKQDTMWHTSWTDKPELHPHWIKIDLNGTKTINKFAYTPRTGASNGTIYNYVLIITDLEGNEKQVAKGVWAANADVKYAEFDAVEATAIKLQVDGNDDKASKGGYGSAAEINIFEVAQKPSANELAENIKVIAPVKAEDTKVSIPVITGFDIVISNSSNPDVIGIDGSITRPENDTVVTLTLKVKETDAKSVKAAGTEATTNVDVLVTGTKTSDVEAESVTLDQTSADLTVGGELLLNAVVKPDIATNKAVTWSSDKPGTATVENGRVKALAAGEARITAATANGKTADCVINVKEKEEPEVILPAEVRLNIPSAEFTVGDQIQLTASVLPANAADKTITWKSDKPEVATVANGWVKGIAAGTAKITATSVNGKTAVCVITVKAQPQNLPTGVSLNKKTASVKLNKTLTLSAVVQPSNADNKTVKWTSDNTYVATVENGVVKAVNAGTARITAATVNGHKATCTITVPGTKISKAKVSLASSKTHTGKALKPSVKVTYGKNTLKKNTDYTVSYKNNINPGTASVTITGKGKYYGTINKTFAIKAAEGKTYTVGKGKYKVTDASAKNKTVTFMAPVKKTYSSFSVPSKVKIGNDTYKVTAVAKNAFKKNTKLTKLTIGSNVKTIGSYAFYGASQLKTLTLKTTGLNSVGKNAFKKTNAKLTVKVPKSKLADYKKLLKGKGLSGKAKIQK

SEQ ID NO: 28 - DNA, 2535 bp, Robinsoniella peoriensis
tcaccattgagcgctgcggcagaaagtggcacaggaaccagattagtgaaagggcaaacggggtatttgacagaggaacaggctatccggaaccaggagcagacaaccgaagaaagggagcagaagttaaccggggaagagacagcagaggttttgatggaaggtacaaaagacagcgggattgtacagacagaagaagtacagacaaaagaaatgcagacagaagatgcgcagacagaagaagtacagacagaagaaatgcagacagaagatgcgcagacaaaagaagtacagacagaagaaatgcagacagaagatgcgcagacagaagaagtacagacaaaagaagaaccggcagaagaaacacacatgaaagaaatacagacgcaagggacaaagaaagcgtcagataggaacggaaaggcaagggtaactgaaattctggaagatgcccaggatccagcaaaccggattgtgtatctgtcagacctgcaatggaagtcagaaaatcatacagtagatagcgagctgcctaccagaaaggataagtcctttggcggcggaaaaattacgctaaaagtggatggaacggtaacagaatttgataaggggattggaacacagacagattccaccattgtgtacgatctggagggaaagggatatacaaagtttgaaacttacgtgggtgtagactacagccagaaagaaaacattccgggggaagtctgcgacgtaaaattcagggtgaaaattgatgacaagattgtatcagaaaccggtgtactggatccgctttcgaatgcggttaagatttctgttaacatacccgatacagccaaaactttaacattatacgcggataaagtaacggaaacttggtctgatcacgccaattgggcagatgcaaaattttatcaggcactgccggaacccgaaaatgttgcattcaaaaaaacggtagtgacacgaaagacatcagataattcggaggctcctgttaatccggattcagcagttaacagttctaaggctgttgacggtgttattgacagctccagttattttgattttggagatcaggcaaatagcggagccgtaagggagtcactctatatggaggtagatttaaaagggagctatttactgtccgatatacaactgtggagatactggaaagatggcagaacttatgcagctactgcaattgtagtagctgaggatgagaactttgaaaatgcagcagttatctataactcggatacgacgggagaaatacatcacctgggagcaggaagtgatatgctctatgcagaaacagaaagtggcaagacatttccggtaccggaaaatacaaaagcaaggtatatcagagtttatacatatggtgttaatgggacatcaggcgtaacaaatcacattgtagaattaaaggtgaatgcttacgtatttggagatgaaatcttaccggaaaagccggatgacagcaagattttcccaaatgcagttaatccgctgaagctacagggaccgggcacgaatgatcaggtaacccacccggatgttacggtgtttgatgagccgtggaatgggtataaatactggatggcatatacaccgaataaaccgggaagttcctattttgaaaatccctgtatagctgcatccaacgatggcgtaaactgggagtttcctgcccagaaccctgtacagccgcgctatgacagtgaaatagaaaatcaaaatgaacataactgtgataccgatattgtatatgacccggtaaatgaccggttgattatgtactgggaatgggcacaggatgaggcggttaatggtaaaacacatcgttctgaaatcagataccgtgtttcttatgatgggattaactggggagtggaagacaaaactggtgttttgatgactggaccaacggatcatggctgcgccattgccacagaaggcgaaagatattcagacctttctccaaccgtagtatatgataaaacagaaaaaatctacaaaatgtgggcaaatgatgccggagatgtaggatatgaaaacaaacagaataacaaagtatggtatcggacatcccaagacgggatcagcaattggtcggataagacttacgtggagaattttcttggagtaaatgaagacgggctgcagatgtatccatggcaccaggatatccagtgggtagaggaatttcaggaatattgggcacttcagcaggcatttccggcaggaagcggaccggataattcttccctgcgtttctcgaaatccaaagatggtcttcattgggagccggtatctgaaaaagctttaattacagtaggggcacccgggacctgggatgcaggacagatataccgttctactttctggtatgagccaggtggggcaaaaggaaacggaacattccatatctggtatgctgcattggcggaaggccagtctcactgggatataggatatacatctgcaaactatgcagatgccatgtacaaattaacgggaagcagatga

SEQ ID NO: 29 - protein, 864 aa, Robinsoniella peoriensis
MGSSHHHHHHSSGLVPRGSHSPLSAAAESGTGTRLVKGQTGYLTEEQAIRNQEQTTEEREQKLTGEETAEVLMEGTKDSGIVQTEEVQTKEMQTEDAQTEEVQTEEMQTEDAQTKEVQTEEMQTEDAQTEEVQTKEEPAEETHMKEIQTQGTKKASDRNGKARVTEILEDAQDPANRIVYLSDLQWKSENHTVDSELPTRKDKSFGGGKITLKVDGTVTEFDKGIGTQTDSTIVYDLEGKGYTKFETYVGVDYSQKENIPGEVCDVKFRVKIDDKIVSETGVLDPLSNAVKISVNIPDTAKTLTLYADKVTETWSDHANWADAKFYQALPEPENVAFKKTVVTRKTSDNSEAPVNPDSAVNSSKAVDGVIDSSSYFDFGDQANSGAVRESLYMEVDLKGSYLLSDIQLWRYWKDGRTYAATAIVVAEDENFENAAVIYNSDTTGEIHHLGAGSDMLYAETESGKTFPVPENTKARYIRVYTYGVNGTSGVTNHIVELKVNAYVFGDEILPEKPDDSKIFPNAVNPLKLQGPGTNDQVTHPDVTVFDEPWNGYKYWMAYTPNKPGSSYFENPCIAASNDGVNWEFPAQNPVQPRYDSEIENQNEHNCDTDIVYDPVNDRLIMYWEWAQDEAVNGKTHRSEIRYRVSYDGINWGVEDKTGVLMTGPTDHGCAIATEGERYSDLSPTVVYDKTEKIYKMWANDAGDVGYENKQNNKVWYRTSQDGISNWSDKTYVENFLGVNEDGLQMYPWHQDIQWVEEFQEYWALQQAFPAGSGPDNSSLRFSKSKDGLHWEPVSEKALITVGAPGTWDAGQIYRSTFWYEPGGAKGNGTFHIWYAALAEGQSHWDIGYTSANYADAMYKLTGSR

SEQ ID NO: 30 - DNA, 3246 bp, Robinsoniella peoriensis
gctgagactgcaacagaagaaaatgcggcgctggaaaaaacagttacattgcataagagcgatggaacagaactgccggaggattatcgaaatccccaaagaccagctaccatggcggtagatggtattattgacgatacaggagagtacaactattgcgatttcggtaaagacggtgataaagcagccctgtatatgcaggtggaccttggaggtctgtatgatttaagcagagtcaatatgtggagatactggaaagacagcagaacttacgatgcaacagtaattaccacatctgagagcggcgatttcacagatgaagcagtcatatataattcagacaggtcgaatgtacatggatttggggcaggaggagatgaacgctacgcagagactgcctccggacatgaattcccagtaccggacggtacaaaggcacaggcagtacgcgtatatgtatttggcagccaaaacggtactacaaaccacatcaatgaattgcaggtctggggaactccccatacagagaatccggatgtaaattcttatcaggtgacaattccacagggaaatggatatcaggtaataccttatgaaaatgacccgacgacagtggaagaaggcggttctttccgttttcaggtactgattgactccgataatggttacagcgcaaccagtgcggtaaaagcaaatggagtaagtctggaggcagttgacagtgtttataccattgagaacattactgaagatcaggtaatcaccattgaaggcgtacataaagcacagtatgaagtgaaattcccggaaaatccacagggatacagtgttgagattcagaatgaaggaagtacaacggtagactataatggttctgtcagttttaagcttattatagacgaagcttataatgaatccgtaccggttgtaaaagcaaacggcggtgcagctttgggaaaagatgagctcggtgtatatacaattgcaaatatccaggacgatattacggttacagttgagggtatccaggaaaataccgtagtaaagacaaaaacaatgtacttgtctgatatggattggaagagtgctgcaaatgcagtaggtgcaacaggagaaaaagacactccaacaaaggacctgaatcatttacagcagcagatgaaattattggtaaacggagcagagaagtcttttgataaaggaattggagttcagacggattcttctatcgtttatgatctggaagacaaaggctacacttctttccacaccctggcaggcgttgattattcagcaatggaatatgtagacggagaaggctgtgatatccagtttaaagtatatctggatgatgtcgtagtatttgacagcggagtagttgatgcatctgatgaggctcaggaagttaatgttgctataacatcagagaataaagaactaaaactggaagctaaaatggttaaagagccttataatgactggggaaactgggcagatgccagctttgaaatggcttatcccgaaccgtctaatgtggctttaaataaaacagttaccgttaagaaaacagcggataactcagactctgaagtaaattccagcagaccgggatcaatggctgtagatggaatcattggacctacatcagattctaactattgtgattttggacaggatggggataatacttcccgttatctgcaggtagatttaggggatgtttatgaacttacccagattaatatgtttagatactgggcagatggcagagtatataatggtactgtaattgcagtttccgaaaacgcagactttagtaatccaacttttatttataattcagataaagcagacaaacacggacttggcgcaggcagtgatgacacttatggagaaacccagagtggaaaattattcgaagttccggcgggaaccatgggacagtatgtccgtgtgtatatggctggttccaacaaaggtacaacgaaccatatcgctgaattacaggtaatgggttataatttcaatacagaaccaaaaccatatgaagcaaatgcatttgaaaatgcagaagtttatttagatatgccaactcatttccaggatctggattccaataaaaacgacgatggaagcttaaagcacattggcggacaggtgacacatcctgatatccaggtatttgaccaaccgtggaacggttataaatactggatgatttacacaccaaatacaatgatcacttcccagtatgaaaatccatatatcgtagcatctgaagatggacagacatgggtagaaccggaagggatttccaatccaattgaaccagaaccgccatcaaccagatttcataactgtgatgcagatctgttatacgactctgtcaatgaccgtttacttgcttactggaactgggcagatgacggcggcggaattgatgacgaattaaaagatcagaactgtcagattcgtctgagaatttcttatgatggaattaactggggagttccttacgacaaagacggcaatattgccacaacagctgatactgtagtaagaatggaaacaggagataaggatttcattcctgcaatcagcgaaaaagaccgttatggtatgctttccccaacatttacctatgacgatttccgcggcatatatacaatgtgggcacaaaactcgggtgatgcgggatacaaccagtccggaaagttcatcgaaatgagatggtctgaggatggaataaactggtctgaaccacaaaaagtgaataatttccttggaaaagatgagaatggcagacagctttggccatggcatcaggatattcagtatatccctgagctacaggaatattggggactgtcccagtgtttctctacatctaatcccgatggatccgtattatacctgaccaagtccagagatggtgtcaactgggagcaggcaggaacacagccggtattaagggcaggaaaatcaggtacctgggatgatttccagatttaccgttctaccttctattatgataatcagtcagacagccctactggtgggaaatttagaatctggtacagtgcactgcaggcaaatacttcaggcaagaccgttttggctcctgatggaacagtgtctcttcaggttggaagccaggataccaggatctggcgtatcgggtatacagaaaatgactacatggaagtcatgaaagctctgacccagaataaaaactatgaagaatga

SEQ ID NO: 31 - protein, 1101 aa, Robinsoniella peoriensis
MGSSHHHHHHSSGLVPRGSHAETATEENAALEKTVTLHKSDGTELPEDYRNPQRPATMAVDGIIDDTGEYNYCDFGKDGDKAALYMQVDLGGLYDLSRVNMWRYWKDSRTYDATVITTSESGDFTDEAVIYNSDRSNVHGFGAGGDERYAETASGHEFPVPDGTKAQAVRVYVFGSQNGTTNHINELQVWGTPHTENPDVNSYQVTIPQGNGYQVIPYENDPTTVEEGGSFRFQVLIDSDNGYSATSAVKANGVSLEAVDSVYTIENITEDQVITIEGVHKAQYEVKFPENPQGYSVEIQNEGSTTVDYNGSVSFKLIIDEAYNESVPVVKANGGAALGKDELGVYTIANIQDDITVTVEGIQENTVVKTKTMYLSDMDWKSAANAVGATGEKDTPTKDLNHLQQQMKLLVNGAEKSFDKGIGVQTDSSIVYDLEDKGYTSFHTLAGVDYSAMEYVDGEGCDIQFKVYLDDVVVFDSGVVDASDEAQEVNVAITSENKELKLEAKMVKEPYNDWGNWADASFEMAYPEPSNVALNKTVTVKKTADNSDSEVNSSRPGSMAVDGIIGPTSDSNYCDFGQDGDNTSRYLQVDLGDVYELTQINMFRYWADGRVYNGTVIAVSENADFSNPTFIYNSDKADKHGLGAGSDDTYGETQSGKLFEVPAGTMGQYVRVYMAGSNKGTTNHIAELQVMGYNFNTEPKPYEANAFENAEVYLDMPTHFQDLDSNKNDDGSLKHIGGQVTHPDIQVFDQPWNGYKYWMIYTPNTMITSQYENPYIVASEDGQTWVEPEGISNPIEPEPPSTRFHNCDADLLYDSVNDRLLAYWNWADDGGGIDDELKDQNCQIRLRISYDGINWGVPYDKDGNIATTADTVVRMETGDKDFIPAISEKDRYGMLSPTFTYDDFRGIYTMWAQNSGDAGYNQSGKFIEMRWSEDGINWSEPQKVNNFLGKDENGRQLWPWHQDIQYIPELQEYWGLSQCFSTSNPDGSVLYLTKSRDGVNWEQAGTQPVLRAGKSGTWDDFQIYRSTFYYDNQSDSPTGGKFRIWYSALQANTSGKTVLAPDGTVSLQVGSQDTRIWRIGYTENDYMEVMKALTQNKNYEE

SEQ ID NO: 32 - protein, 528 aa, Clostridium tertium
HSGQYWLVFQPDNDVLQTKTNPSSMKQSANNNPYNYNILPNSFPIGTGYNAYKGDVSFYATFKEASSQAIPQNSWALKYVDSEETTGENGRATNAFDGNNNTIWHTKYSGGNAAPMPHEIQIDLRGVYNINQINYLPRQDGGTNGTIKDYEVYLSLDGVNWGQPISKGTFESNSTEKIVKFNETKSRYVKLKALSEINNKQFTTVADLKVFGWEISKIEKPLQNAETYLNIPTYDGLNQSTHPDVKYFKNGWNGYKYWMIMTPNRTGSSVAENPSILASDDGINWEVPAGVTNPIAPMPQVGHNCDVDMIYNEATDELWVYWVESDDITKGWVKLIKSKDGVNWSSQQVVVDDNRAKYSTLSPSIIFKDNKYYMWSVNTGNSGWNNQSNKVELRESSDGVNWSNPTVVNTLAQDGSQIWHVNVEYIPSKNEYWAIYPAYKNGTGSDKTELYYAKSSDGVNWTTYKNPILSKGTSGKWDDMEIYRSCFVYDEDTNMIKVWYGAVSQNPQIWKIGFTENDYDKFIEGLTQ

SEQ ID NO: 33 - protein, 449 aa, Ruthenibacterium lactatiformans
HEETDLLVNGGFETGDSTGWNWFNNAVVDSAAPHSGNYCAKVAKNSSYEQVVTVSPDTKYVLTGWAKSEGSSVMTLGVKNYGGQETFSATLSADYQQLAVTFTTGPNAQTATIYGYRQNSGSGAGYFDDVELTAVQDFAPYQPLANAIAPQAIPTYDGANQPTHPSVVKFEQPWNGYLYWMAMTPYPFNDGSYENPSIVASNDGENWIVPEGVSNPLAGTPSPGHNCDVDLVYVPASDELRMYYVEADDIISSRVKMISSRDGVHWSEPQVVMQDLVRKYSILSPSIEILPDGTYMMWYVDTGNAGWNSQNNQVKYRTSADGIKWSGAVTCTDFVQPGYQIWHIDVHYDTSSGAYYAVYPAYPNGTDCDHCNLFFAVNRTGKQWETFSRPILKPSTEGGWDDFCIYRSSMLIDDGMLKVWYGAKKQEDSSWHTGLTMRDFSEFMKILER

SEQ ID NO: 34 - protein, 845 aa, Robinsoniella peoriensis
HSPLSAAAESGTGTRLVKGQTGYLTEEQAIRNQEQTTEEREQKLTGEETAEVLMEGTKDSGIVQTEEVQTKEMQTEDAQTEEVQTEEMQTEDAQTKEVQTEEMQTEDAQTEEVQTKEEPAEETHMKEIQTQGTKKASDRNGKARVTEILEDAQDPANRIVYLSDLQWKSENHTVDSELPTRKDKSFGGGKITLKVDGTVTEFDKGIGTQTDSTIVYDLEGKGYTKFETYVGVDYSQKENIPGEVCDVKFRVKIDDKIVSETGVLDPLSNAVKISVNIPDTAKTLTLYADKVTETWSDHANWADAKFYQALPEPENVAFKKTVVTRKTSDNSEAPVNPDSAVNSSKAVDGVIDSSSYFDFGDQANSGAVRESLYMEVDLKGSYLLSDIQLWRYWKDGRTYAATAIVVAEDENFENAAVIYNSDTTGEIHHLGAGSDMLYAETESGKTFPVPENTKARYIRVYTYGVNGTSGVTNHIVELKVNAYVFGDEILPEKPDDSKIFPNAVNPLKLQGPGTNDQVTHPDVTVFDEPWNGYKYWMAYTPNKPGSSYFENPCIAASNDGVNWEFPAQNPVQPRYDSEIENQNEHNCDTDIVYDPVNDRLIMYWEWAQDEAVNGKTHRSEIRYRVSYDGINWGVEDKTGVLMTGPTDHGCAIATEGERYSDLSPTVVYDKTEKIYKMWANDAGDVGYENKQNNKVWYRTSQDGISNWSDKTYVENFLGVNEDGLQMYPWHQDIQWVEEFQEYWALQQAFPAGSGPDNSSLRFSKSKDGLHWEPVSEKALITVGAPGTWDAGQIYRSTFWYEPGGAKGNGTFHIWYAALAEGQSHWDIGYTSANYADAMYKLTGSR

SEQ ID NO: 35 - protein, 1082 aa, Robinsoniella peoriensis
HAETATEENAALEKTVTLHKSDGTELPEDYRNPQRPATMAVDGIIDDTGEYNYCDFGKDGDKAALYMQVDLGGLYDLSRVNMWRYWKDSRTYDATVITTSESGDFTDEAVIYNSDRSNVHGFGAGGDERYAETASGHEFPVPDGTKAQAVRVYVFGSQNGTTNHINELQVWGTPHTENPDVNSYQVTIPQGNGYQVIPYENDPTTVEEGGSFRFQVLIDSDNGYSATSAVKANGVSLEAVDSVYTIENITEDQVITIEGVHKAQYEVKFPENPQGYSVEIQNEGSTTVDYNGSVSFKLIIDEAYNESVPVVKANGGAALGKDELGVYTIANIQDDITVTVEGIQENTVVKTKTMYLSDMDWKSAANAVGATGEKDTPTKDLNHLQQQMKLLVNGAEKSFDKGIGVQTDSSIVYDLEDKGYTSFHTLAGVDYSAMEYVDGEGCDIQFKVYLDDVVVFDSGVVDASDEAQEVNVAITSENKELKLEAKMVKEPYNDWGNWADASFEMAYPEPSNVALNKTVTVKKTADNSDSEVNSSRPGSMAVDGIIGPTSDSNYCDFGQDGDNTSRYLQVDLGDVYELTQINMFRYWADGRVYNGTVIAVSENADFSNPTFIYNSDKADKHGLGAGSDDTYGETQSGKLFEVPAGTMGQYVRVYMAGSNKGTTNHIAELQVMGYNFNTEPKPYEANAFENAEVYLDMPTHFQDLDSNKNDDGSLKHIGGQVTHPDIQVFDQPWNGYKYWMIYTPNTMITSQYENPYIVASEDGQTWVEPEGISNPIEPEPPSTRFHNCDADLLYDSVNDRLLAYWNWADDGGGIDDELKDQNCQIRLRISYDGINWGVPYDKDGNIATTADTVVRMETGDKDFIPAISEKDRYGMLSPTFTYDDFRGIYTMWAQNSGDAGYNQSGKFIEMRWSEDGINWSEPQKVNNFLGKDENGRQLWPWHQDIQYIPELQEYWGLSQCFSTSNPDGSVLYLTKSRDGVNWEQAGTQPVLRAGKSGTWDDFQIYRSTFYYDNQSDSPTGGKFRIWYSALQANTSGKTVLAPDGTVSLQVGSQDTRIWRIGYTENDYMEVMKALTQNKNYEE

SEQ ID NO: 36 - protein, 986 aa, Clostridium tertium
HYNLIDNISVEKLDTDISQANENVFLNGNGIALEVDNRGATCIYLVDENGVKTKATTSLDTADFSGYPIIGGQKIRDFVIISKNLEENINSILGVGNRLTIISKSSSTNLIRKIVFETSNSNPGAIYSTVSYKAESNDLLVDSFHENEYTMSLGQGPFLAYQGCADQQGANTIVNVTNGYNHNSGQNNYSVGVPFSYVYNSVGGIGIGDASTSRREFKLPIIGKDNTVSLGMEWNGQTLKKGAETAIGTSVITTTNGDYYSGLKSYAEVMKDKGISAPASIPDIAYDSRWESWGFEFDFTIEKIVNKLDELKAMGIKQITLDDGWYTYAGDWKLSPQKFPNGNADMKYLTDEIHKRGMTAILWWRPVDGGINSKLVSEHPEWFIKNSQGNMVRLPGPGGGNGGTAGYALCPNSEGSIQHHKDFVTVALEEWGFDGFKEDYVWGIPKCYDSSHKHSSLSDTLENQYKFYEAIYEQSIAINPDTFIELCNCGTPQDFYSTPYVNHAPTADPISRVQTRTRVKAFKAIFGDDFPVTTDHNSVWLPSALGTGSVMITKHTTLSSSDREQYNKYFGLARDLELAKGEFIGNLYKYGIDPLESYVIRKGEDIYYSFYKDNSSYSGNIEIKGLDSNATYRIEDYVNNRVIARGVKGPTATINTSFTDNLLVRAIPDDTPAEVTTFDVGNNTILSSTDSGNSKYLNAVSTTLEKTATIDSLSIYIGNNSENGKLQIAIYDDNNGKPGTKKAYVEEFVPTKNSWNTKKVVNSVTLPSGQYWLVFQPDNDVLQTKTNPSSMKQSANNNPYNYNILPNSFPIGTGYNAYKGDVSFYATFKEASSQAIPQNSWALKYVDSEETTGENGRATNAFDGNNNTIWHTKYSGGNAAPMPHEIQIDLRGVYNINQINYLPRQDGGTNGTIKDYEVYLSLDGVNWGQPISKGTFESNSTEKIVKFNETKSRYVKLKALSEINNKQFTTVADLKVFGWEISKIEK

SEQ ID NO: 37 - protein, 1262 aa, Robinsoniella peoriensis
HGNGLEVKASPREVAQITGNGVSVTFFQEDGTVQLSCIEDDGNTAFMTRNSEVSYPVVGGEEVTDFSDFQCEVQENVTGAAGAGSRMTITSISSGRGIQRSVVIETVDEVKGLLHISSSYRAEEEVDADEFIDSRFSLDNPSDTVWSYNGGGEGAQSRYDTLQKIDLSDGESFYRENLQNQTAAGIPVADIYGKDGGITVGDASVTRRQLSTPVNERNGTAYVSVKHPGAVITQRETEISQSFVNVHRGDYYSGLRGYADGMKQIGFTTLSREQIPESSYDLRWESWGWEFDWTVELIINKLDELKEMGIKQITLDDGWYNAAGEWGLNNWKLPNGALDMRHLTDAIHERGMTAVLWWRPCDGGREDSALFKEHPEYFIKNQDGSFGKLAGPGQWNSFLGSCGYALCPLSEGAVQSQVDFINRAMNEWGFDGFKSDYVWSLPKCYSQDHHHEYPEESTEQQAVFYRAVYEAMTDNDPNAFHLLCNCGTPQDYYSLPYVTQVPTADPTSVDQTRRRVKAYKALCGDYFPVTTDHNEVWYPSTIGTGAILIEKRDLSGWEEEEYAKWLKIAQENQLHKGTFIGDLYSYGYDPYETYTVYKDGIMYYAFYKDGNRYRPSGNPDIELKGLEDGKLYRIVDYVNNQVVATNVTSSNAVFSYPFSDYLLVKAVEISEPDTDGPGPVPDPEGAVTVEENDPELVYTGDWVREENDGYHGGGARYTKEAEASVELAFYGTGAAWYGQHDVNFGSARIYIDGTYVKTVSCMGEPGINIKLFEISGLDLASHRIKIECETPVIDIDRLTYIKGEEVPAKVMTADLRALTVIANQYDMNSFADGNYKDQLGVSLVRANQLLAADDVTQGAVNEEQKYLLNAMLKIRKKVDKSWIGLPGPIPQDIQTENISRDNLAKVISYTGQLDRDEIIPAIKEQLNDSYDKAVSIAERQDASQPEIDRAWAELMNAVQYSSYIRGSKEELLSLLDEYGKVDTTVYKDAALFIESLEAAKKVYQDENAMDGEISDCIKQLRDAKDQLQLKDPVDPPKPDPDPDPKPDPTPDPGPDPKPDPTPDPTPDPKPNPTPTPDPTPEPALKKPEQVSGLKSKAETDYLTVSWKKLNNAESYKVYIYKSGKWRLAGKTTKTSIKIKKLVSGTKYTVKVAAVNKAGQGKYSSQVYTAAKPKKVKLKSVSRYRTSKVKLNYGKVKAGGYEIWMKNGKGSYKKAATSTKTTAIKSGLKKGKTYYFKVRAYVKNKNQVIYGSFSNIKKYKMVL

SEQ ID NO: 38 - DNA, 32 bp, Artificial Sequence
  Primer FpGalNAcDeAc_withoutSignalP_fw
atggtctcgccatgcagactccagcgagtccg

SEQ ID NO: 39 - DNA, 34 bp, Artificial Sequence
  Primer FpGalNAcDeAc_D1min_rv
atggtctcgattcttacgtcgtgtagccggggtc

SEQ ID NO: 40 - DNA, 41 bp, Artificial Sequence
  Primer FpGalNAcDeAc_D1ext_rv
atggtctcgattcttaatcactggaggtatatttcacgacc

SEQ ID NO: 41 - DNA, 38 bp, Artificial Sequence
  Primer FpGalNAcDeAc_D1+2_rv
atggtctcgattcttacgcaggctcgattggaccatac

SEQ ID NO: 42 - DNA, 34 bp, Artificial Sequence
  Primer FpGalNAcDeAc_D2ext_fw
atggtctcgccatgatgtggcgacggtggatgag

SEQ ID NO: 43 - DNA, 41 bp, Artificial Sequence
  Primer FpGalNAcDeAc_rv
atggtctcgattcttattctcccacatacgaaaaatagtcg

SEQ ID NO: 44 - DNA, 39 bp, Artificial Sequence
  Primer FpGalNase_withoutSignalP_fw
atggtctcgccatcgtggtaaaaagttcatatcactcac

SEQ ID NO: 45 - DNA, 43 bp, Artificial Sequence
  Primer FpGalNase_truncA_rv
atggtctcgattcttatgcgttagtggtataagtcaaatagtc

SEQ ID NO: 46 - DNA, 40 bp, Artificial Sequence
  Primer FpGalNase_rv
atggtctcgattcttattccgaaatttccaccgctttaac

SEQ ID NO: 47 - DNA, 50 bp, Artificial Sequence
  Primer Ct5757_fw
atggtctcgccattataatttaattgataatattagtgttgaaaaattag

SEQ ID NO: 48 - DNA, 38 bp, Artificial Sequence
  Primer Ct5757_rv
atggtctcgattcttattgtgttaaaccctcaataaac

SEQ ID NO: 49 - DNA, 45 bp, Artificial Sequence
  Primer Ct5757_GalNase_rv
atggtctcgattcttaatgagtactttgatttaatccatcataag

SEQ ID NO: 50 - DNA, 37 bp, Artificial Sequence
  Primer Ct5757_DeAcase_fw
atggtctcgccattcagggcaatattggttagttttc

SEQ ID NO: 51 - DNA, 35 bp, Artificial Sequence
  Primer Rp1021_fw
atggtctcgccatgggaacggattagaggtgaaag

SEQ ID NO: 52 - DNA, 45 bp, Artificial Sequence
  Primer Rp1021_rv
atggtctcgattctcataataccattttgtatttctttatattgg

SEQ ID NO: 53 - DNA, 37 bp, Artificial Sequence
  Primer Rl8755_fw
atggtctcgccatgaagaaaccgatttgcttgtaaac

SEQ ID NO: 54 - DNA, 42 bp, Artificial Sequence
  Primer Rl8755_rv
atggtctcgattcttagcgttccaatattttcataaattcag

SEQ ID NO: 55 - DNA, 32 bp, Artificial Sequence
  Primer Rp3671_fw
atggtctcgccattcaccattgagcgctgcgg

SEQ ID NO: 56 - DNA, 44 bp, Artificial Sequence
  Primer Rp3671_rv
atggtctcgattcttatgactttgttttaacatttacagacttg

SEQ ID NO: 57 - DNA, 38 bp, Artificial Sequence
  Primer Rp3672_fw
atggtctcgccatgctgagactgcaacagaagaaaatg

SEQ ID NO: 58 - DNA, 39 bp, Artificial Sequence
  Primer Rp3672_rv
atggtctcgattcttatttctgaatttttgccttgccag

SEQ ID NO: 59 - protein, 6 aa, Artificial Sequence
  Protein Tag Sequence AU1 epitope
DTYRYI

SEQ ID NO: 60 - protein, 6 aa, Artificial Sequence
  Protein Tag Sequence AU5 epitope
TDFYLK

SEQ ID NO: 61 - protein, 15 aa, Artificial Sequence
  Protein Tag Sequence AviTag
GLNDIFEAQKIEWHE

SEQ ID NO: 62 - protein, 11 aa, Artificial Sequence
  Protein Tag Sequence T7-tag
MASMTGGQQMG

SEQ ID NO: 63 - protein, 14 aa, Artificial Sequence
  Protein Tag Sequence V5-tag
GKPIPNPLLGLDST

SEQ ID NO: 64 - protein, 6 aa, Artificial Sequence
  Protein Tag Sequence B-tag
QYPALT

SEQ ID NO: 65 - protein, 26 aa, Artificial Sequence
  Protein Tag Sequence Calmodulin-tag
KRRWKKNFIAVSAANRFKKISSSGAL

SEQ ID NO: 66 - protein, 4 aa, Artificial Sequence
  Protein Tag Sequence C-tag
EPEA

SEQ ID NO: 67 - protein, 23 aa, Artificial Sequence
  Protein Tag Sequence DogTag
DIPATYEFTDGKHYITNEPIPPK

SEQ ID NO: 68 - protein, 10 aa, Artificial Sequence
  Protein Tag Sequence E2 epitope
SSTSSDFRDR

SEQ ID NO: 69 - protein, 13 aa, Artificial Sequence
  Affinity Tag Sequence E-tag
GAPVPYPDPLEPR

SEQ ID NO: 70 - protein, 7 aa, Artificial Sequence
  Affinity Tag Sequence FLAG-tag
DYKDDDK

SEQ ID NO: 71 - protein, 6 aa, Artificial Sequence
  Protein Tag Sequence EE-tag(1)
EYMPME

SEQ ID NO: 72 - protein, 6 aa, Artificial Sequence
  Protein Tag Sequence EE-tag (2)
EFMPME

SEQ ID NO: 73 - protein, 9 aa, Artificial Sequence
  Protein Tag Sequence HA-tag
YPYDVPDYA

SEQ ID NO: 74 - protein, 19 aa, Artificial Sequence
  Protein Tag Sequence HAT
KDHLIHNVHKEFHAHAHNK

SEQ ID NO: 75 - protein, 6 aa, Artificial Sequence
  Protein Tag Sequence HQ-tag
HQHQHQ

SEQ ID NO: 76 - protein, 12 aa, Artificial Sequence
  Protein Tag Sequence HN-tag
HNHNHNHNHNHN

SEQ ID NO: 77 - protein, 8 aa, Artificial Sequence
  Protein Tag Sequence HSV epitope
QPELAPED

SEQ ID NO: 78 - protein, 16 aa, Artificial Sequence
  Protein Tag Sequence Isopep-tag
TDKDMTITFTNKKDAE

SEQ ID NO: 79 - protein, 11 aa, Artificial Sequence
  Protein Tag Sequence KT3 epitope
KPPTPPPEPET

SEQ ID NO: 80 - protein, 11 aa, Artificial Sequence
  Protein Tag Sequence Myc epitope
CEQKLISEEDL

SEQ ID NO: 81 - protein, 10 aa, Artificial Sequence
  Protein Tag Sequence Myc-tag
EQKLISEEDL

SEQ ID NO: 82 - protein, 18 aa, Artificial Sequence
  Protein Tag Sequence NE-tag
TKENPRSNQEESYDDNES

SEQ ID NO: 83 - protein, 5 aa, Artificial Sequence
  Protein Tag Sequence Arg-tag
RRRRR

SEQ ID NO: 84 - protein, 5 aa, Artificial Sequence
  Protein Tag Sequence Asp-tag
DDDDD

SEQ ID NO: 85 - protein, 4 aa, Artificial Sequence
  Protein Tag Sequence Cys-tag
CCCC

SEQ ID NO: 86 - protein, 6 aa, Artificial Sequence
  Protein Tag Sequence Glu-tag
EEEEEE

SEQ ID NO: 87 - protein, 6 aa, Artificial Sequence
  Protein Tag Sequence His-tag
HHHHHH

SEQ ID NO: 88 - protein, 11 aa, Artificial Sequence
  Protein Tag Sequence Phe-tag
FFFFFFFFFFF

SEQ ID NO: 89 - protein, 9 aa, Artificial Sequence
  Protein Tag Sequence Rho1D4-tag
TETSQVAPA

SEQ ID NO: 90 - protein, 9 aa, Artificial Sequence
  Protein Tag Sequence S1-tag
NANNPDWDF

SEQ ID NO: 91 - protein, 15 aa, Artificial Sequence
  Protein Tag Sequence S-tag
KETAAAKFERQHMDS

SEQ ID NO: 92 - protein, 13 aa, Artificial Sequence
  Protein Tag Sequence Softtag 1
SLAELLNAGLGGS

SEQ ID NO: 93 - protein, 8 aa, Artificial Sequence
  Protein Tag Sequence Softtag 3
TQDPSRVG

SEQ ID NO: 94 - protein, 13 aa, Artificial Sequence
  Protein Tag Sequence Spy-tag
AHIVMVDAYKPTK

SEQ ID NO: 95 - protein, 38 aa, Artificial Sequence
  Protein Tag Sequence SBP-tag
MDEKTTGWRGGHVVEGLAGELEQLRARLEHHPQGQREP

SEQ ID NO: 96 - protein, 8 aa, Artificial Sequence
  Protein Tag Sequence Strep-tag (1)
WSHPQFEK

SEQ ID NO: 97 - protein, 9 aa, Artificial Sequence
  Protein Tag Sequence Strep-tag (2)
AWAHPQPGG

SEQ ID NO: 98 - protein, 8 aa, Artificial Sequence
  Protein Tag Sequence Strep-tag II
WSHPQFEK

SEQ ID NO: 99 - protein, 13 aa, Artificial Sequence
  Protein Tag Sequence Sdy-tag
DPIVMIDNDKPIT

SEQ ID NO: 100 - protein, 12 aa, Artificial Sequence
  Protein Tag Sequence SnoopTag
KLGDIEFIKVNK

SEQ ID NO: 101 - protein, 12 aa, Artificial Sequence
  Protein Tag Sequence SnoopTagJr
KLGSIEFIKVNK

SEQ ID NO: 102 - protein, 12 aa, Artificial Sequence
  Protein Tag Sequence Spot-tag
PDRVRAVSHWSS

SEQ ID NO: 103 - protein, 6 aa, Artificial Sequence
  Protein Tag Sequence TC-tag
CCPGCC

SEQ ID NO: 104 - protein, 10 aa, Artificial Sequence
  Protein Tag Sequence Ty-tag
EVHTNQDPLD

SEQ ID NO: 105 - protein, 6 aa, Artificial Sequence
  Protein Tag Sequence Universal
HTTPHH

SEQ ID NO: 106 - protein, 11 aa, Artificial Sequence
  Protein Tag Sequence VSV-tag
YTDIEMNRLGK

SEQ ID NO: 107 - protein, 14 aa, Artificial Sequence
  Protein Tag Sequence V5-tag
GKPIPNPLLGLDST

SEQ ID NO: 108 - protein, 8 aa, Artificial Sequence
  Protein Tag Sequence Xpress tag
DLYDDDDK